(19) United States
(12) Patent Application Publication
(10) Pub. No.: US 2023/0227405 A1
(43) Pub. Date: Jul. 20, 2023

(54) 5-OXOPYRROLIDINE-3-CARBOXAMIDES AS NAV1.8 INHIBITORS

(71) Applicants: Ashok ARASAPPAN, Kenilworth, NJ (US); Ian M. BELL, West Point, PA (US); Jason M. COX, Flemington, NJ (US); Michael J. KELLY, III, West Point, PA (US); Mark E. LAYTON, West Point, PA (US); Hong LIU, Kenilworth, NJ (US); Jian LIU, Edison, NJ (US); Akshay A. SHAH, West Point, PA (US); Michael D. VANHEYST, Harleysville, PA (US); MERCK SHARP & DOHME CORP., RAHWAY, NJ (US)

(72) Inventors: Ashok Arasappan, Bridgewater, NJ (US); Ian M. Bell, Harleysville, PA (US); Jason Michael Cox, Flemington, NJ (US); Michael J. Kelly, III, Paoli, PA (US); Mark E. Layton, Harleysville, PA (US); Hong Liu, Hillsborough, NJ (US); Jian Liu, Edison, NJ (US); Akshay A. Shah, Robbinsville, NJ (US); Michael David VanHeyst, Harleysville, PA (US)

(73) Assignee: Merck Sharp & Dohme LLC, Rahway, NJ (US)

(21) Appl. No.: 18/009,310

(22) PCT Filed: Jun. 14, 2021
(86) PCT No.: PCT/US2021/037160

§ 371 Date: Dec. 8, 2022

Related U.S. Application Data
(60) Provisional application No. 63/040,465, filed on Jun. 17, 2020.

(51) Int. Cl. C07D 207/277; C07D 401/12; C07D 417/12

(52) U.S. Cl. CPC C07D 207/277; C07D 401/12; C07D 417/12

(57) ABSTRACT

Novel compounds of the structural formula (I), and the pharmaceutically acceptable salts thereof, are inhibitors of Naᵥ1.8 channel activity and may be useful in the treatment, prevention, management, amelioration, control and suppression of diseases mediated by Naᵥ1.8 channel activity. The compounds of the present invention may be useful in the treatment, prevention or management of pain disorders, cough disorders, acute itch disorders, and chronic itch disorders.

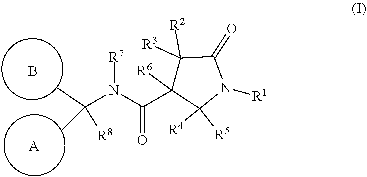

### BACKGROUND OF THE INVENTION

Voltage-gated sodium channels (VGSC) mediate the selective influx of sodium ions in excitable cells and play a central role in initiating and propagating action potentials (Yu et al., Genome Biology 4:207 (2003)). Voltage-gated sodium channels are ubiquitous in the central and peripheral nervous system where they play a central role in the initiation and propagation of action potentials, and also in skeletal and cardiac muscle where the action potential triggers cellular contraction (Goldin et al., Ann N Y Acad Sci. 1999 Apr. 30; 868:38-50). Alterations in VGSC function or their expression can profoundly affect normal cell excitability (Huang et al., J Neurosci. 2013 Aug. 28; 33 (35):14087-97; Emery et al., J Neurosci. 2015 May 20; 35(20):7674-81; Kist et al., PLoS One. 2016 Sep. 6; 11(9):e0161789; and Schreiber et al., World J Diabetes. 2015 Apr. 15; 6(3):432-44).

Voltage-gated sodium channels are multimeric complexes characterized by one α-subunit, which forms an ion-conducting aqueous pore, and at least one β-subunit that modifies the kinetics and voltage-dependence of the channel gating. Nine different α-subunits have been identified and characterized in mammalian voltage-gated sodium channels, including Naᵥ1.8, also known as SNS, PN3 or Naᵥ1.8 (Goldin et al., Neuron. 2000 November; 28 (2):365-8).

Expression of sodium channels can be tissue specific. Naᵥ1.8 voltage-gated sodium ion channels are expressed primarily in sensory neurons, which are responsible for conveying information from the periphery (e.g. skin, muscle and joints) to the central nervous system via the spinal cord. Sodium channels are integral to this process as sodium channel activity is required for initiation and propagation of action potentials triggered by noxious stimuli (thermal, mechanical and chemical) activating peripheral nociceptors (Catterall et al., Nat Chem Biol. 2017 Apr. 13; 13(5):455-463). An increase in VGSC protein level at the cell surface or an alteration in activity of the VGSC channels can result in disease states such as migraine, neurodegeneration following ischemia, epilepsies, and chronic neuropathic and inflammatory pain states. Gain of function mutations in Naᵥ1.7, Naᵥ1.8, and Naᵥ1.9 manifest in a variety of pain syndromes where patients experience spontaneous pain without an external stimulus (Bennett et al., Lancet Neurol. 2014 June; 13(6):587-99; Huang et al., J Neurosci. 2013 Aug. 28; 33(35):14087-97; Kist et al., PLoS One. 2016 Sep. 6; 11(9):e0161789; Emery et al., J Neurosci. 2015 May 20; 35(20):7674-81; and Schreiber et al., World J Diabetes. 2015 Apr.

Naᵥ1.8 voltage-gated sodium ion channels are believed to play a role in various maladies, including neuropathic pain, chronic itch, and inflammatory pain perception (Belkouch et al., J Neuroinflammation. 2014 Mar. 7; 11:45; Coward et al., Pain. 2000 March; 85(1-2):41-50; Yiangou et al., FEBS Lett. 2000 Feb. 11; 467(2-3):249-52; Black et al., Ann Neurol. 2008 December; 64(6):644-53; Bird et al., Br J Pharmacol. 2015 May; 172(10):2654-70; Liu et al., Neuron. 2010 Nov. 4; 68(3):543-56; and Zhao et al., J Clin Invest. 2013).

Large portions of the voltage gated sodium ion channels are conserved among the various subtypes, therefore there is a potential for producing serious side effects when utilizing therapeutic agents that do not demonstrate subtype selectivity. Therefore, therapeutic agents suitable for use in addressing nociception, cough, or itch disorders, require specificity in their action, for example, discriminating between action upon Naᵥ1.5 sodium ion channels, thought to be important in regulation of cardiac function, and action upon Naᵥ1.8 sodium ion channels, thought to be central in inflammatory nociception, or itch and disorders arising from dysfunctional and/or upregulated Naᵥ1.8 sodium ion channels.

Accordingly, it is believed that inhibitors of Naᵥ1.8 voltage-gated sodium ion channel activity may useful to treat or prevent diseases, disorders and conditions involving Naᵥ1.8 receptors and/or stemming specifically from dysfunction of Naᵥ1.8 voltage-gated sodium ion channels (Han et al., J Neurol Neurosurg Psychiatry 2014 May; 85(5):499-505), including but not limited to, migraine, neurodegeneration following ischemia, epilepsy, inflammatory pain, spontaneous pain, acute pain, preoperative pain, perioperative pain, post-operative pain, neuropathic pain, chronic itch, and itch disorders.

There remains a need for potent Naᵥ1.8 sodium ion channel activity inhibitors with selective activity for Naᵥ1.8 sodium ion channels. As a result, the compounds of the present invention are useful for the treatment and prevention of diseases, disorders and conditions involving Naᵥ1.8 receptors and Naᵥ1.8 voltage-gated sodium ion channels.

The role of Naᵥ1.8 sodium ion channels is discussed in: Bennett et al., Physical Medicine and Rehabilitation Clinics of North America, 2001, 12(2):447-459; Meissner et al., Br J Sports Med. 2018 May; 52(10):642-650; Legroux-Crespel et al., Neurology. 2016 Feb. 2; 86(5):473-83; and Flaxman et al., Lancet, 380:2163-2196 (2012).

Compounds useful to treat Naᵥ1.8 sodium ion channel related conditions are disclosed in: ACS Med. Chem. Lett. 2015, 6, 650; BJP 2015, 172, 2654; PNAS 2007, 104, 8520; J. Med. Chem. 2008, 51, 407; JPET 2008, 324, 1204; and Neuropharmacology 2010, 59, 201.

Naᵥ1.8 compounds are also disclosed in: WO 2009/049180, WO 2009/049181, WO 2009/049183, WO 2014/120808; WO 2014/120815; WO 2014/120820; WO 2015/010065; and WO 2015/089361; WO 2017/209322; U.S. Pat. Nos. 8,519,137; 9,051,270; 9,108,903; 9,163,042; 9,783,501; WO 2020/092667; WO2019/014352; WO2018/213426; U.S. Pat. No. 8,629,149; and WO2011/026240.

### SUMMARY OF THE INVENTION

The present invention relates to novel compounds of structural formula I:

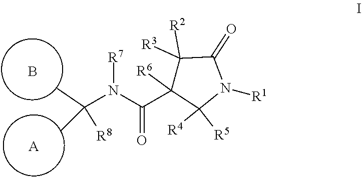

and pharmaceutically acceptable salts thereof. The compounds of structural formula I, and embodiments thereof, are inhibitors of Naᵥ1.8 sodium ion channel activity (or Naᵥ1.8 inhibitors) and may be useful in the treatment and prevention of diseases, disorders and conditions mediated by Naᵥ1.8 sodium ion channel activity, such as nociception, osteoarthritis, peripheral neuropathy, inherited erythromelalgia, multiple sclerosis, asthma, itch, atopy, allergic or contact dermatitis, renal failure, cholestasis, pruritus, acute itch, chronic itch, migraine, neurodegeneration following ischemia, epilepsy, pain, inflammatory pain, spontaneous pain, acute pain, acute pain due to fractures, musculoskeletal damage, pancreatitis and renal colic, pen-operative pain, post-operative pain, neuropathic pain, postherpetic neuralgia, trigeminal neuralgia, diabetic neuropathy, chronic lower back pain, phantom limb pain, sciatica, pain caused by 2° or 3° burn injury, optic neuritis, pain resulting from cancer and chemotherapy, chronic pelvic pain, pain syndromes, and complex regional pain syndromes. In one embodiment of the present invention, the condition, disease or disorder is a pain disorder, an acute pain disorder or chronic pain disorder. In another embodiment of the present invention, the condition, disease or disorder is an acute pain disorder.

The present invention also relates to pharmaceutical compositions comprising the compounds of the present invention and a pharmaceutically acceptable carrier.

The present invention also relates to methods for the treatment, management, prevention, alleviation, amelioration, suppression or control of disorders, diseases, and conditions that may be responsive to inhibition of Naᵥ1.8 sodium ion channel activity in a subject in need thereof by administering the compounds and pharmaceutical compositions of the present invention.

The present invention also relates to the use of compounds of the present invention for manufacture of a medicament useful in treating diseases, disorders and conditions that may be responsive to the inhibition of Naᵥ1.8 sodium ion channel activity.

The present invention is also concerned with treatment or prevention of these diseases, disorders and conditions by administering the compounds of the present invention in combination with a therapeutically effective amount of another agent that may be useful to treat the disease, disorder and condition. The invention is further concerned with processes for preparing the compounds of this invention.

### DETAILED DESCRIPTION OF THE INVENTION

The present invention is concerned with novel compounds of structural Formula I:

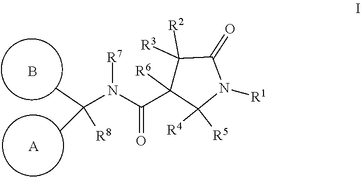

or a pharmaceutically acceptable salt thereof, wherein
one of A and B is selected from:
1) aryl, and
2) heteroaryl,
   wherein aryl and heteroaryl are unsubstituted or substituted with one to five substituents selected from R^{a},
   and the other of A and B is selected from:
1) aryl,
2) heteroaryl,
3) —C₁₋₆alkyl-aryl,
4) —C₃₋₈cycloalkyl-aryl,
5) —C₂₋₈cycloheteroalkyl-aryl,
6) —C₁₋₆alkyl-heteroaryl,
7) —C₃₋₈cycloalkyl-heteroaryl,
8) —C₂₋₈cycloheteroalkyl-heteroaryl,
9) —C₁₋₆alkyl-O-aryl,
10) —C₁₋₆alkyl-O-heteroaryl,
11) —C₃₋₁₂cycloalkyl,
12) —C₂₋₁₂cycloheteroalkyl,
13) —C₁₋₆alkyl-C₃₋₁₂cycloalkyl,
14) —C₁₋₆alkyl-C₂₋₁₂cycloheteroalkyl,
15) —C₁₋₆alkyl-O—C₃₋₁₂cycloalkyl,
16) —C₁₋₆alkyl-O—C₂₋₁₂cycloheteroalkyl,
17) —C₀₋₆alkyl-aryl fused to C₄₋₆cycloalkyl or C₄₋₆cycloheteroalkyl containing 1-3 heteroatoms independently selected from O, S and N(R^{h})₂,
18) —C₀₋₆alkyl-aryl fused to C₄₋₆cycloalkenyl or C₄₋₆cycloheteroalkenyl containing 1-3 heteroatoms independently selected from O, S and N(R^{h})₂,
19) —C₀₋₆alkyl-heteroaryl fused to C₄₋₆cycloalkyl or C₄₋₆cycloheteroalkyl containing 1-3 heteroatoms independently selected from O, S and N(R^{h})₂, and
20) —C₀₋₆alkyl-heteroaryl fused to C₄₋₆cycloalkenyl or C₄₋₆cycloheteroalkenyl containing 1-3 heteroatoms independently selected from O, S and N(R^{h})₂,
   wherein alkyl, cycloalkyl, cycloheteroalkyl, cycloalkenyl, aryl and heteroaryl are unsubstituted or substituted with one to five substituents selected from R^{b};
   R¹ is selected from the group consisting of:
1) hydrogen,
2) —C₁₋₆alkyl,
3) —C₃₋₆alkenyl,
4) —C₃₋₆alkynyl,
5) —C₃₋₁₀cycloalkyl,
6) —C₂₋₁₀cycloheteroalkyl,
7) —C₁₋₆alkyl-O—C₁₋₆alkyl-,
8) —(CH₂)ₛC(O)R^{j},
9) —(CH₂)ₛC(O)NR^{e}R^{j},
10) —(CH₂)ₙNR^{e}C(O)R^{j},
11) —(CH₂)ₙNR^{e}C(O)OR^{j},
12) —(CH₂)ₙNR^{e}C(O)N(R^{e})₂,
13) —(CH₂)ₙNR^{e}C(O)NR^{e}R^{j},
14) —(CH₂)ₙNR^{e}S(O)ₘR^{j},
15) —(CH₂)ₙNR^{e}S(O)mN(R^{e})₂,
16) —(CH₂)ₙNR^{e}S(O)mNR^{e}R^{j}, and
17) —(CH₂)ₙNR^{e}R^{j},
   wherein each CH₂, alkyl, alkenyl, alkynyl, cycloalkyl and cycloheteroalkyl is unsubstituted or substituted with one to five substituents selected from R^{c};
   R² is selected from the group consisting of:
1) hydrogen,
2) —C₁₋₆alkyl,
3) —C₂₋₆alkenyl,
4) —C₂₋₆alkynyl,
5) —C₃₋₁₀cycloalkyl,
6) —C₂₋₁₀cycloheteroalkyl,
7) —C₁₋₆alkyl-O—C₁₋₆alkyl-,
8) —(CH₂)ₛC(O)R^{j},
9) —(CH₂)ₛC(O)NR^{e}R^{j},
10) —(CH₂)ₛNR^{e}C(O)R^{j},
11) —(CH₂)ₛNR^{e}C(O)OR^{j},
12) —(CH₂)ₛNR^{e}C(O)N(R^{e})₂,
13) —(CH₂)ₛNR^{e}C(O)NR^{e}R^{j},
14) —(CH₂)ₛNR^{e}S(O)ₘR^{j},
15) —(CH₂)ₛNR^{e}S(O)mN(R^{e})₂,
16) —(CH₂)ₛNR^{e}S(O)mNR^{e}R^{j}, and
17) —(CH₂)ₛNR^{e}R^{j},
   wherein each CH₂, alkyl, alkenyl, alkynyl, cycloalkyl, and cycloheteroalkyl is unsubstituted or substituted with one to five substituents selected from R^{d}, and wherein R² and R³ and the carbon atom they are connected to can form a —C₃₋₅cycloalkyl ring;
   R³ is selected from the group consisting of:
1) hydrogen,
2) —C₁₋₆alkyl,
3) —C₂₋₆alkenyl,
4) —C₂₋₆alkynyl,
5) —C₃₋₁₀cycloalkyl,
6) —C₂₋₁₀cycloheteroalkyl,
7) —C₁₋₆alkyl-O—C₁₋₆alkyl-,
8) —(CH₂)ₛC(O)R^{j},
9) —(CH₂)ₛC(O)NR^{e}R^{j},
10) —(CH₂)ₛNR^{e}C(O)R^{j},
11) —(CH₂)ₛNR^{e}C(O)OR^{j},
12) —(CH₂)ₛNR^{e}C(O)N(R^{e})₂,
13) —(CH₂)ₛNR^{e}C(O)NR^{e}R^{j},
14) —(CH₂)ₛNR^{e}S(O)ₘR^{j},
15) —(CH₂)ₛNR^{e}S(O)mN(R^{e})₂,
16) —(CH₂)ₛNR^{e}S(O)mNR^{e}R^{j}, and
17) —(CH₂)ₛNR^{e}R^{j},
   wherein each CH₂, alkyl, alkenyl, alkynyl, cycloalkyl, and cycloheteroalkyl is unsubstituted or substituted with one to five substituents selected from R^{f};
   R⁴ is selected from the group consisting of:
1) hydrogen,
2) —C₁₋₆alkyl,
3) —C₂₋₆alkenyl,
4) —C₂₋₆alkynyl,
6) —C₂₋₁₀cycloheteroalkyl,
7) —C₁₋₆alkyl-O—C₁₋₆alkyl-,
8) —(CH₂)ₛC(O)R^{j},
9) —(CH₂)ₛC(O)NR^{e}R^{j},
10) —(CH₂)ₛNR^{e}C(O)R^{j},
11) —(CH₂)ₛNR^{e}C(O)OR^{j},
12) —(CH₂)ₛNR^{e}C(O)N(R^{e})₂,
13) —(CH₂)ₛNR^{e}C(O)NR^{e}R^{j},
14) —(CH₂)ₛNR^{e}S(O)ₘR^{j},
15) —(CH₂)ₛNR^{e}S(O)mN(R^{e})₂,
16) —(CH₂)ₛNR^{e}S(O)mNR^{e}R^{j}, and
17) —(CH₂)ₛNR^{e}R^{j},
   wherein each CH₂, alkyl, alkenyl, alkynyl, cycloalkyl, and cycloheteroalkyl is unsubstituted or substituted with one to five substituents selected from R^{g};
   R⁵ is selected from the group consisting of:
1) hydrogen,
2) —C₁₋₆alkyl,
3) —C₂₋₆alkenyl,
4) —C₂₋₆alkynyl,
5) —C₃₋₁₀cycloalkyl,
6) —C₂₋₁₀cycloheteroalkyl,
7) —C₁₋₆alkyl-O—C₁₋₆alkyl-,
8) —(CH₂)ₛC(O)R^{j},
9) —(CH₂)ₛC(O)NR^{e}R^{j},
10) —(CH₂)ₛNR^{e}C(O)R^{j},
11) —(CH₂)ₛNR^{e}C(O)OR^{j},
12) —(CH₂)ₛNR^{e}C(O)N(R^{e})₂,
13) —(CH₂)ₛNR^{e}C(O)NR^{e}R^{j},
14) —(CH₂)ₛNR^{e}S(O)ₘR^{j},
15) —(CH₂)ₛNR^{e}S(O)mN(R^{e})₂,
16) —(CH₂)ₛNR^{e}S(O)mNR^{e}R^{j}, and
17) —(CH₂)ₛNR^{e}R^{j},
   wherein each CH₂, alkyl, alkenyl, alkynyl, cycloalkyl, and cycloheteroalkyl is unsubstituted or substituted with one to five substituents selected from R^{g}, and
   wherein R⁵ and R⁴ and the carbon atom they are connected to can form a —C₃₋₅cycloalkyl ring, or wherein R⁵ and R⁶ and the carbon atoms they are connected to can form a —C₃₋₅cycloalkyl ring;
   R⁶ is selected from the group consisting of:
1) hydrogen, and
2) —C₁₋₆alkyl,
   wherein each alkyl is unsubstituted or substituted with one to five halogen substituents;
   R⁷ is selected from the group consisting of:
1) hydrogen,
2) —C₁₋₆alkyl,
3) —C₃₋₆cycloalkyl, and
4) —C₂₋₆cycloheteroalkyl,
   wherein each alkyl, cycloalkyl and cycloheteroalkyl is unsubstituted or substituted with one to five halogen substituents;
   R⁸ is selected from the group consisting of:
1) hydrogen,
2) —C₁₋₆alkyl,
3) —C₂₋₆alkenyl, and
4) —C₂₋₆alkynyl,
   wherein each alkyl, alkenyl and alkynyl is unsubstituted or substituted with one to five halogen substituents;
   each R^{a} is independently selected from the group consisting of:
1) —CF₃,
2) —OCF₃,
3) —CHF₂,
4) —OCHF₂,
5) —CH₂CF₃,
6) —OCH₂CF₃,
7) —CF₂CH₃
8) CN,
9) oxo,
10) halogen,
11) —S(O)₂C₁₋₆alkyl,
12) —C₁₋₆alkyl,
13) —C₂₋₆alkenyl,
14) —C₂₋₆alkynyl,
15) —C₃₋₆cycloalkyl,
16) —C₂₋₆cycloheteroalkyl,
17) aryl,
18) heteroaryl,
19) —C₁₋₆alkyl-aryl,
20) —C₁₋₆alkyl-heteroaryl,
21) —C₁₋₆alkyl-C₃₋₆cycloalkyl,
22) —C₁₋₆alkyl-C₂₋₆cycloheteroalkyl,
23) —C₂₋₆alkenyl-C₃₋₆cycloalkyl,
24) —C₂₋₆alkenyl-C₂₋₆cycloheteroalkyl,
25) —C₂₋₆alkenyl-aryl,
26) —C₂₋₆alkenyl-heteroaryl,
27) —C₂₋₆alkynyl-C₃₋₆cycloalkyl,
28) —C₂₋₆alkynyl-C₂₋₆cycloheteroalkyl,
29) —C₂₋₆alkynyl-aryl,
30) —C₂₋₆alkynyl-heteroaryl,
31) —OH,
32) —(CH₂)ₚ—OC₁-6alkyl,
33) —(CH₂)ₚ—OC₂₋₆alkenyl,
34) —(CH₂)ₚ—OC₂₋₆alkynyl,
35) —(CH₂)ₚ—OC₃₋₆cycloalkyl,
36) —(CH₂)ₚ—OC₂₋₆heterocycloalkyl,
37) —(CH₂)ₚ—O-aryl,
38) —(CH₂)ₚ—O-heteroaryl,
39) —OC₁₋₆alkyl-C₃₋₆cycloalkyl,
40) —OC₁₋₆alkyl-C₂₋₆ heterocycloalkyl,
41) —OC₁₋₆alkyl-aryl,
42) —OC₁₋₆alkyl-heteroaryl,
43) —S(O)ₘRⁱ,
44) —C₁₋₆alkyl-S(O)ₘRⁱ,
45) —N(R^{k})₂, and
46) —NR^{k}R^{L},
   wherein each R^{a} is unsubstituted or substituted with one to six substituents selected from halogen, CF₃, OH, C₁₋₆alkyl, and OC₁₋₆alkyl;
   each R^{b} is independently selected from the group consisting of:
1) —CF₃,
2) —OCF₃,
3) —CHF₂,
4) —OCHF₂,
5) —CH₂CF₃,
6) —OCH₂CF₃,
7) —CF₂CH₃
8) CN,
9) oxo,
10) halogen,
11) —S(O)₂C₁₋₆alkyl,
12) —C₁₋₆alkyl,
13) —C₂₋₆alkenyl,
14) —C₂₋₆alkynyl,
15) —O—C₁₋₆alkyl,
16) —C₃₋₆cycloalkyl,
17) —O—C₃₋₆cycloalkyl,
18) —C₂₋₆cycloheteroalkyl,
19) aryl,
20) heteroaryl,
21) —C₁₋₆alkyl-aryl,
22) —C₁₋₆alkyl-heteroaryl,
23) —C₁₋₆alkyl-C₃₋₆cycloalkyl,
24) —C₁₋₆alkyl-C₂₋₆cycloheteroalkyl,
25) —C₂₋₆alkenyl-C₃₋₆cycloalkyl,
26) —C₂₋₆alkenyl-C₂₋₆cycloheteroalkyl,
27) —C₂₋₆alkenyl-aryl,
28) —C₂₋₆alkenyl-heteroaryl,
29) —C₂₋₆alkynyl-C₃₋₆cycloalkyl,
30) —C₂₋₆alkynyl-C₂₋₆cycloheteroalkyl,
31) —C₂₋₆alkynyl-aryl,
32) —C₂₋₆alkynyl-heteroaryl,
33) —OH,
34) —(CH₂)_{q}—OC₁₋₆alkyl,
35) —(CH₂)_{q}—OC₂₋₆alkenyl,
36) —(CH₂)_{q}—OC₂₋₆alkynyl,
37) —(CH₂)_{q}—OC₃₋₆cycloalkyl,
38) —(CH₂)_{q}—OC₂₋₆heterocycloalkyl,
39) —(CH₂)_{q}—O-aryl,
40) —(CH₂)_{q}—O-heteroaryl,
41) —OC₁₋₆alkyl-C₃₋₆cycloalkyl,
42) —OC₁₋₆alkyl-C₂₋₆ heterocycloalkyl,
43) —OC₁₋₆alkyl-aryl,
44) —OC₁-6alkyl-heteroaryl,
45) —S(O)ₘR^{j},
46) —C₁₋₆alkyl-S(O)ₘR^{j},
47) —C(O)R^{L}, and
48) —NR^{k}R^{L},
   wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl;
   R^{c} is selected from:
1) —C₁₋₆alkyl,
2) OH,
3) halogen, and
4) —OC₁₋₆alkyl,
   wherein alkyl can be unsubstituted or substituted with one to three halogens;
   R^{d} is selected from:
1) —C₁₋₆alkyl,
2) OH,
3) halogen, and
4) —OC₁₋₆alkyl,
   wherein alkyl can be unsubstituted or substituted with one to three halogens;
   R^{e} is selected from:
1) hydrogen, and
2) C₁₋₆alkyl;
   R^{f} is selected from:
1) —C₁₋₆alkyl,
2) OH,
3) halogen, and
4) —OC₁₋₆alkyl,
   wherein alkyl can be unsubstituted or substituted with one to three halogens;
   R^{g} is selected from:
1) —C₁₋₆alkyl,
2) OH,
3) halogen, and
4) —OC₁₋₆alkyl,
   wherein alkyl can be unsubstituted or substituted with one to three halogens; R^{h} is selected from:
1) hydrogen, and
2) C₁₋₆alkyl;
   Rⁱ is selected from:
1) hydrogen,
2) C₁₋₆alkyl,
3) C₃₋₆cycloalkyl,
4) aryl, and
5) heteroaryl;
   R^{j} is selected from:
1) hydrogen,
2) C₁₋₆alkyl,
3) C₃₋₆alkenyl,
4) C₃₋₆alkynyl,
5) C₃₋₆cycloalkyl,
6) C₂₋₅cycloheteroalkyl,
7) aryl, and
8) heteroaryl;
   R^{k} is selected from:
1) hydrogen, and
2) C₁₋₆alkyl;
   R^{L} is selected from:
1) hydrogen,
2)
3) C₃₋₆cycloalkyl,
4) aryl, and
5) heteroaryl;
   m is independently selected from 0 to 2;
   n is independently selected from 2 to 6;
   p is independently selected from 0 to 3;
   q is independently selected from 0 to 3;
   r is independently selected from 0 to 2; and
   s is independently selected from 0 to 6.

The invention has numerous embodiments, which are summarized below. The invention includes the compounds as shown, and also includes individual diastereoisomers, enantiomers, and epimers of the compounds, and mixtures of diastereoisomers and/or enantiomers thereof including racemic mixtures.

In one embodiment of the present invention, one of A and B is selected from: aryl, and heteroaryl, wherein aryl and heteroaryl are unsubstituted or substituted with one to five substituents selected from R^{a}, and the other of A and B is selected from: aryl, heteroaryl, —C₁₋₆alkyl-aryl, —C₃₋₈cycloalkyl-aryl, —C₂₋₈cycloheteroalkyl-aryl, —C₁₋₆alkyl-heteroaryl, —C₃₋₈cycloalkyl-heteroaryl, —C₂₋₈cycloheteroalkyl-heteroaryl, —C₁₋₆alkyl-O-aryl, —C₁₋₆alkyl-O-heteroaryl, —C₃₋₁₂cycloalkyl, —C₂₋₁₂cycloheteroalkyl, —C₁₋₆alkyl-C₃₋₁₂cycloalkyl, —C₁₋₆alkyl-C₂₋₁₂cycloheteroalkyl, —C₁₋₆alkyl-O—C₃₋₁₂cycloalkyl, —C₁₋₆alkyl-O—C₂₋₁₂cycloheteroalkyl, —C₀₋₆alkyl-aryl fused to C₄₋₆cycloalkyl or C₄₋₆cycloheteroalkyl containing 1-3 heteroatoms independently selected from O, S and N(R^{h})₂, —C₀₋₆alkyl-aryl fused to C₄₋₆cycloalkenyl or C₄₋₆cycloheteroalkenyl containing 1-3 heteroatoms independently selected from O, S and N(R^{h})₂, —C₀₋₆alkyl-heteroaryl fused to C₄₋₆cycloalkyl or C₄₋₆cycloheteroalkyl containing 1-3 heteroatoms independently selected from O, S and N(R^{h})₂, and —C₀₋₆alkyl-heteroaryl fused to C₄₋₆cycloalkenyl or C₄₋₆cycloheteroalkenyl containing 1-3 heteroatoms independently selected from O, S and N(R^{h})₂, wherein alkyl, cycloalkyl, cycloheteroalkyl, cycloalkenyl, aryl and heteroaryl are unsubstituted or substituted with one to five substituents selected from R^{b}.

In another embodiment, one of A and B is selected from: aryl, and heteroaryl, wherein aryl and heteroaryl are unsubstituted or substituted with one to five substituents selected from R^{a}, and the other of A and B is selected from: aryl, heteroaryl, —C₁₋₆alkyl-aryl, —C₁₋₆alkyl-heteroaryl, —C₁₋₆alkyl-O-aryl, —C₁₋₆alkyl-O-heteroaryl, C₃₋₁₂cycloalkyl, and C₂₋₁₂cycloheteroalkyl, wherein alkyl, cycloalkyl, cycloheteroalkyl, aryl and heteroaryl are unsubstituted or substituted with one to five substituents selected from R^{b}.

In another embodiment, one of A and B is selected from: aryl, and heteroaryl, wherein aryl and heteroaryl are unsubstituted or substituted with one to five substituents selected from R^{a}, and the other of A and B is selected from: aryl, heteroaryl, —C₁₋₆alkyl-aryl, —C₁₋₆alkyl-O-aryl, and C₃₋₁₂cycloalkyl, wherein alkyl, cycloalkyl, aryl and heteroaryl are unsubstituted or substituted with one to five substituents selected from R^{b}.

In another embodiment, one of A and B is aryl, wherein aryl is unsubstituted or substituted with one to five substituents selected from R^{a}, and the other of A and B is selected from: aryl, heteroaryl, C₁₋₆alkyl-aryl, C₁₋₆alkyl-O-aryl, and C₃₋₁₂cycloalkyl, wherein alkyl, cycloalkyl, aryl and heteroaryl are unsubstituted or substituted with one to five substituents selected from R^{b}.

In another embodiment, one of A and B is aryl, wherein aryl is unsubstituted or substituted with one to five substituents selected from R^{a}, and the other of A and B is aryl, wherein aryl is unsubstituted or substituted with one to five substituents selected from R^{b}.

In another embodiment of the present invention, A is selected from the group consisting of: aryl, and heteroaryl, wherein aryl and heteroaryl are unsubstituted or substituted with one to five substituents selected from R^{a}. In a class of this embodiment, A is substituted with 0-4 substituents selected from R^{a}. In another class of this embodiment, A is substituted with 0-3 substituents selected from R^{a}. In another class of this embodiment, A is substituted with 0-2 substituents selected from R^{a}.

In another embodiment of the present invention, A is aryl, wherein aryl is unsubstituted or substituted with one to five substituents selected from R^{a}. In a class of this embodiment, A is substituted with 0-4 substituents selected from R^{a}. In another class of this embodiment, A is substituted with 0-3 substituents selected from R^{a}. In another class of this embodiment, A is substituted with 0-2 substituents selected from R^{a}.

In another embodiment of the present invention, A is phenyl, wherein phenyl is unsubstituted or substituted with one to five substituents selected from R^{a}. In a class of this embodiment, A is substituted with 0-4 substituents selected from R^{a}. In another class of this embodiment, A is substituted with 0-3 substituents selected from R^{a}. In another class of this embodiment, A is substituted with 0-2 substituents selected from R^{a}.

In another embodiment of the present invention, A is independently selected from the group consisting of: aryl, heteroaryl, —C₁₋₆alkyl-aryl, —C₃₋₈cycloalkyl-aryl, —C₂₋₈cycloheteroalkyl-aryl, —C₁₋₆alkyl-heteroaryl, —C₃₋₈cycloalkyl-heteroaryl, —C₂₋₈cycloheteroalkyl-heteroaryl, —C₁₋₆alkyl-O-aryl, —C₁₋₆alkyl-O-heteroaryl, —C₃₋₁₂cycloalkyl, —C₂₋₁₂cycloheteroalkyl, —C₁₋₆alkyl-C₃₋₁₂cycloalkyl, —C₁₋₆alkyl-C₂₋₁₂cycloheteroalkyl, —C₁₋₆alkyl-O—C₃₋₁₂cycloalkyl, —C₁₋₆alkyl-O—C₂₋₁₂cycloheteroalkyl, —C₀₋₆alkyl-aryl fused to C₄₋₆cycloalkyl or C₄₋₆cycloheteroalkyl containing 1-3 heteroatoms independently selected from O, S and N(R^{h})₂, —C₀₋₆alkyl-aryl fused to C₄₋₆cycloalkenyl or C₄₋₆cycloheteroalkenyl containing 1-3 heteroatoms independently selected from O, S and N(R^{h})₂, —C₀₋₆alkyl-heteroaryl fused to C₄₋₆cycloalkyl or C₄₋₆cycloheteroalkyl containing 1-3 heteroatoms independently selected from O, S and N(R^{h})₂, and —C₀₋₆alkyl-heteroaryl fused to C₄₋₆cycloalkenyl or C₄₋₆cycloheteroalkenyl containing 1-3 heteroatoms independently selected from O, S and N(R^{h})₂, wherein alkyl, cycloalkyl, cycloheteroalkyl, cycloalkenyl, aryl and heteroaryl are unsubstituted or substituted with one to five substituents selected from R^{b}. In a class of this embodiment, A is substituted with 0-4 substituents selected from R^{b}. In another class of this embodiment, A is substituted with 0-3 substituents selected from R^{b}. In another class of this embodiment, A is substituted with 0-2 substituents selected from R^{b}.

In another embodiment of the present invention, A is independently selected from the group consisting of: aryl, heteroaryl, —C₁₋₆alkyl-aryl, —C₃₋₈cycloalkyl-aryl, —C₂₋₈cycloheteroalkyl-aryl, —C₁₋₆alkyl-heteroaryl, —C₃₋₈cycloalkyl-heteroaryl, —C₂₋₈cycloheteroalkyl-heteroaryl, —C₁₋₆alkyl-O-aryl, —C₁₋₆alkyl-O-heteroaryl, —C₃₋₁₂cycloalkyl, —C₂₋₁₂cycloheteroalkyl, —C₁₋₆alkyl-C₃₋₁₂cycloalkyl, —C₁₋₆alkyl-C₂₋₁₂cycloheteroalkyl, —C₁₋₆alkyl-O—C₃₋₁₂cycloalkyl, and —C₁₋₆alkyl-O—C₂₄₂cycloheteroalkyl, wherein alkyl, cycloalkyl, cycloheteroalkyl, aryl and heteroaryl are unsubstituted or substituted with one to five substituents selected from R^{b}. In a class of this embodiment, A is substituted with 0-4 substituents selected from R^{b}. In another class of this embodiment, A is substituted with 0-3 substituents selected from R^{b}. In another class of this embodiment, A is substituted with 0-2 substituents selected from R^{b}.

In another embodiment of the present invention, A is independently selected from the group consisting of: aryl, heteroaryl, —C₁₋₆alkyl-aryl, —C₁₋₆alkyl-heteroaryl, —C₁₋₆alkyl-O-aryl, —C₁₋₆alkyl-O-heteroaryl, —C₃₋₁₂cycloalkyl, and —C₂₋₁₂cycloheteroalkyl, wherein alkyl, cycloalkyl, cycloheteroalkyl, aryl and heteroaryl are unsubstituted or substituted with one to five substituents selected from R^{b}. In a class of this embodiment, A is substituted with 0-4 substituents selected from R^{b}. In another class of this embodiment, A is substituted with 0-3 substituents selected from R^{b}. In another class of this embodiment, A is substituted with 0-2 substituents selected from R^{b}.

In another embodiment of the present invention, A is independently selected from the group consisting of: aryl, heteroaryl, —C₁₋₆alkyl-aryl, —C₁₋₆alkyl-O-aryl, and —C₃₋₁₂cycloalkyl, wherein alkyl, cycloalkyl, aryl and heteroaryl are unsubstituted or substituted with one to five substituents selected from R^{b}. In a class of this embodiment, A is substituted with 0-4 substituents selected from R^{b}. In another class of this embodiment, A is substituted with 0-3 substituents selected from R^{b}. In another class of this embodiment, A is substituted with 0-2 substituents selected from R^{b}.

In another embodiment of the present invention, A is independently selected from the group consisting of: phenyl, pyridine, thiazole, —(CH₂)₂-phenyl, —CH₂—O-phenyl, and cyclobutane, wherein A is unsubstituted or substituted with one to five substituents selected from R^{b}. In a class of this embodiment, A is substituted with 0-4 substituents selected from R^{b}. In another class of this embodiment, A is substituted with 0-3 substituents selected from R^{b}. In another class of this embodiment, A is substituted with 0-2 substituents selected from R^{b}.

In another embodiment of the present invention, A is aryl, wherein aryl is unsubstituted or substituted with one to five substituents selected from R^{b}. In a class of this embodiment, A is substituted with 0-4 substituents selected from R^{b}. In another class of this embodiment, A is substituted with 0-3 substituents selected from R^{b}. In another class of this embodiment, A is substituted with 0-2 substituents selected from R^{b}.

In another embodiment of the present invention, A is phenyl, wherein phenyl is unsubstituted or substituted with one to five substituents selected from R^{b}. In a class of this embodiment, A is substituted with 0-4 substituents selected from R^{b}. In another class of this embodiment, A is substituted with 0-3 substituents selected from R^{b}. In another class of this embodiment, A is substituted with 0-2 substituents selected from R^{b}.

In another embodiment of the present invention, B is independently selected from the group consisting of: aryl, heteroaryl, —C₁₋₆alkyl-aryl, —C₃₋₈cycloalkyl-aryl, —C₂₋₈cycloheteroalkyl-aryl, —C₁₋₆alkyl-heteroaryl, —C₃₋₈cycloalkyl-heteroaryl, —C₂₋₈cycloheteroalkyl-heteroaryl, —C₁₋₆alkyl-O-aryl, —C₁₋₆alkyl-O-heteroaryl, —C₃₋₁₂cycloalkyl, —C₂₋₁₂cycloheteroalkyl, —C₁₋₆alkyl-C₃₋₁₂cycloalkyl, —C₁₋₆alkyl-C₂₋₁₂cycloheteroalkyl, —C₁₋₆alkyl-O—C₃₋₁₂cycloalkyl, —C₁₋₆alkyl-O—C₂₋₁₂cycloheteroalkyl, —C₀₋₆alkyl-aryl fused to C₄₋₆cycloalkyl or C₄₋₆cycloheteroalkyl containing 1-3 heteroatoms independently selected from O, S and N(R^{h})₂, —C₀₋₆alkyl-aryl fused to C₄₋₆cycloalkenyl or C₄₋₆cycloheteroalkenyl containing 1-3 heteroatoms independently selected from O, S and N(R^{h})₂, —C₀₋₆alkyl-heteroaryl fused to C₄₋₆cycloalkyl or C₄₋₆cycloheteroalkyl containing 1-3 heteroatoms independently selected from O, S and N(R^{h})₂, and —C₀₋₆alkyl-heteroaryl fused to C₄₋₆cycloalkenyl or C₄₋₆cycloheteroalkenyl containing 1-3 heteroatoms independently selected from O, S and N(R^{h})₂, wherein alkyl, cycloalkyl, cycloheteroalkyl, cycloalkenyl, aryl and heteroaryl are unsubstituted or substituted with one to five substituents selected from R^{b}. In a class of this embodiment, B is substituted with 0-4 substituents selected from R^{b}. In another class of this embodiment, B is substituted with 0-3 substituents selected from R^{b}. In another class of this embodiment, B is substituted with 0-2 substituents selected from R^{b}.

In another embodiment of the present invention, B is independently selected from the group consisting of: aryl, heteroaryl, —C₁₋₆alkyl-aryl, —C₃₋₈cycloalkyl-aryl, —C₂₋₈cycloheteroalkyl-aryl, —C₁₋₆alkyl-heteroaryl, —C₃₋₈cycloalkyl-heteroaryl, —C₂₋₈cycloheteroalkyl-heteroaryl, —C₁₋₆alkyl-O-aryl, —C₁₋₆alkyl-O-heteroaryl, —C₃₋₁₂cycloalkyl, —C₂₋₁₂cycloheteroalkyl, —C₁₋₆alkyl-C₃₋₁₂cycloalkyl, —C₁₋₆alkyl-C₂₋₁₂cycloheteroalkyl, —C₁₋₆alkyl-O—C₃₋₁₂cycloalkyl, and —C₁₋₆alkyl-O—C₂₋₁₂cycloheteroalkyl, wherein alkyl, cycloalkyl, cycloheteroalkyl, aryl and heteroaryl are unsubstituted or substituted with one to five substituents selected from R^{b}. In a class of this embodiment, B is substituted with 0-4 substituents selected from R^{b}. In another class of this embodiment, B is substituted with 0-3 substituents selected from R^{b}. In another class of this embodiment, B is substituted with 0-2 substituents selected from R^{b}.

In another embodiment of the present invention, B is independently selected from the group consisting of: aryl, heteroaryl, —C₁₋₆alkyl-aryl, —C₁₋₆alkyl-heteroaryl, —C₁₋₆alkyl-O-aryl, —C₁₋₆alkyl-O-heteroaryl, —C₃₋₁₂cycloalkyl, and —C₂₋₁₂cycloheteroalkyl, wherein alkyl, cycloalkyl, cycloheteroalkyl, aryl and heteroaryl are unsubstituted or substituted with one to five substituents selected from R^{b}. In a class of this embodiment, B is substituted with 0-4 substituents selected from R^{b}. In another class of this embodiment, B is substituted with 0-3 substituents selected from R^{b}. In another class of this embodiment, B is substituted with 0-2 substituents selected from R^{b}.

In another embodiment of the present invention, B is independently selected from the group consisting of: aryl, heteroaryl, —C₁₋₆alkyl-aryl, —C₁₋₆alkyl-O-aryl, and —C₃₋₁₂cycloalkyl, and wherein alkyl, cycloalkyl, aryl and heteroaryl are unsubstituted or substituted with one to five substituents selected from R^{b}. In a class of this embodiment, B is substituted with 0-4 substituents selected from R^{b}. In another class of this embodiment, B is substituted with 0-3 substituents selected from R^{b}. In another class of this embodiment, B is substituted with 0-2 substituents selected from R^{b}.

In another embodiment of the present invention, B is independently selected from the group consisting of: phenyl, pyridine, thiazole, —(CH₂)₂-phenyl, —CH₂—O-phenyl, and cyclobutane, wherein B is unsubstituted or substituted with one to five substituents selected from R^{b}. In a class of this embodiment, B is substituted with 0-4 substituents selected from R^{b}. In another class of this embodiment, B is substituted with 0-3 substituents selected from R^{b}. In another class of this embodiment, B is substituted with 0-2 substituents selected from R^{b}.

In another embodiment of the present invention, B is aryl, wherein aryl is unsubstituted or substituted with one to five substituents selected from R^{b}. In a class of this embodiment, B is substituted with 0-4 substituents selected from R^{b}. In another class of this embodiment, B is substituted with 0-3 substituents selected from R^{b}. In another class of this embodiment, B is substituted with 0-2 substituents selected from R^{b}.

In another embodiment of the present invention, B is phenyl, wherein phenyl is unsubstituted or substituted with one to five substituents selected from R^{b}. In a class of this embodiment, B is substituted with 0-4 substituents selected from R^{b}. In another class of this embodiment, B is substituted with 0-3 substituents selected from R^{b}. In another class of this embodiment, B is substituted with 0-2 substituents selected from R^{b}.

In another embodiment of the present invention, B is selected from the group consisting of: aryl, and heteroaryl, wherein aryl and heteroaryl are unsubstituted or substituted with one to five substituents selected from R^{a}. In a class of this embodiment, A is substituted with 0-4 substituents selected from R^{a}. In another class of this embodiment, A is substituted with 0-3 substituents selected from R^{a}. In another class of this embodiment, A is substituted with 0-2 substituents selected from R^{a}.

In another embodiment of the present invention, B is aryl, wherein aryl is unsubstituted or substituted with one to five substituents selected from R^{a}. In a class of this embodiment, B is substituted with 0-4 substituents selected from R^{a}. In another class of this embodiment, B is substituted with 0-3 substituents selected from R^{a}. In another class of this embodiment, B is substituted with 0-2 substituents selected from R^{a}.

In another embodiment of the present invention, B is phenyl, wherein phenyl is unsubstituted or substituted with one to five substituents selected from R^{a}. In a class of this embodiment, B is substituted with 0-4 substituents selected from R^{a}. In another class of this embodiment, B is substituted with 0-3 substituents selected from R^{a}. In another class of this embodiment, B is substituted with 0-2 substituents selected from R^{a}.

In one embodiment of the present invention, R¹ is selected from the group consisting of: hydrogen, —C₁₋₆alkyl, —C₃₋₆alkenyl, —C₃₋₆alkynyl, —C₃₋₁₀cycloalkyl, —C₂₋₁₀cycloheteroalkyl, —C₁₋₆alkyl-O—C₁₋₆alkyl-, —(CH₂)ₛC(O)R^{j}, —(CH₂)ₛC(O)NR^{e}R^{j}, —(CH₂)ₙNR^{e}C(O)R^{j}, —(CH₂)ₙNR^{e}C(O)OR^{j}, —(CH₂)ₙNR^{e}C(O)N(R^{e})₂, —(CH₂)ₙNR^{e}C(O)NR^{e}R^{j}, —(CH₂)ₙNR^{e}S(O)ₘR^{j}, —(CH₂)ₙNR^{e}S(O)mN(R^{e})₂, —(CH₂)ₙNR^{e}S(O)mNR^{e}R^{j}, and —(CH₂)ₙNR^{e}R^{j}, wherein each CH₂, alkyl, alkenyl, alkynyl, cycloalkyl and cycloheteroalkyl is unsubstituted or substituted with one to five substituents selected from R^{c}.

In another embodiment, R¹ is selected from the group consisting of: hydrogen, —C₁₋₆alkyl, —C₃₋₆alkenyl, —C₃₋₆alkynyl, —C₃₋₁₀cycloalkyl, —C₂₋₁₀cycloheteroalkyl, and —C₁₋₆alkyl-O—C₁₋₆alkyl-, wherein each alkyl, alkenyl, alkynyl, cycloalkyl and cycloheteroalkyl is unsubstituted or substituted with one to five substituents selected from R^{c}.

In another embodiment, R¹ is selected from the group consisting of: hydrogen, —C₁₋₆alkyl, —C₃₋₆alkenyl, —C₃₋₆alkynyl, —C₃₋₁₀cycloalkyl, and —C₂₋₁₀cycloheteroalkyl, wherein each alkyl, alkenyl, alkynyl, cycloalkyl and cycloheteroalkyl is unsubstituted or substituted with one to five substituents selected from R^{c}.

In another embodiment, R¹ is selected from the group consisting of: hydrogen, —C₁₋₆alkyl, —C₃₋₁₀cycloalkyl, and —C₂₋₁₀cycloheteroalkyl, wherein each alkyl, cycloalkyl and cycloheteroalkyl is unsubstituted or substituted with one to five substituents selected from R^{c}.

In another embodiment, R¹ is selected from the group consisting of: hydrogen, and —C₁₋₆alkyl, wherein each alkyl is unsubstituted or substituted with one to five substituents selected from R^{c}. In another embodiment, R¹ is selected from the group consisting of: hydrogen, and —CH₃.

In another embodiment, R¹ is —C₁₋₆alkyl, wherein each alkyl is unsubstituted or substituted with one to five substituents selected from R^{c}. In another embodiment, R¹ is —CH₃. In another embodiment, R¹ is hydrogen.

In one embodiment of the present invention, R² is selected from the group consisting of: hydrogen, —C₁₋₆alkyl, —C₂₋₆alkenyl, —C₂₋₆alkynyl, —C₃₋₁₀cycloalkyl, —C₂₋₁₀cycloheteroalkyl, —C₁₋₆alkyl-O—C₁₋₆alkyl-, —(CH₂)ₛC(O)R^{j}, —(CH₂)ₛC(O)NR^{e}R^{j}, —(CH₂)ₛNR^{e}C(O)R^{j}, —(CH₂)ₛNR^{e}C(O)OR^{j}, —(CH₂)ₛNR^{e}C(O)N(R^{e})₂, —(CH₂)ₛNR^{e}C(O)NR^{e}R^{j}, —(CH₂)ₛNR^{e}S(O)ₘR^{j}, —(CH₂)ₛNR^{e}S(O)mN(R^{e})₂, —(CH₂)ₛNR^{e}S(O)mNR^{e}R^{j}, and —(CH₂)ₛNR^{e}R^{j}, wherein each CH₂, alkyl, alkenyl, alkynyl, cycloalkyl, and cycloheteroalkyl is unsubstituted or substituted with one to five substituents selected from R^{d}, and wherein R² and R³ and the carbon atom they are connected to can form a —C₃₋₅cycloalkyl ring, or wherein R⁵ and R⁶ and the carbon atoms they are connected to can form a —C₃₋₅cycloalkyl ring. In one embodiment of the present invention, R² is selected from the group consisting of: hydrogen, —C₁₋₆alkyl, —C₂₋₆alkenyl, —C₂₋₆alkynyl, —C₃₋₁₀cycloalkyl, —C₂₋₁₀cycloheteroalkyl, —(CH₂)ₛC(O)R^{j}, —(CH₂)ₛC(O)NR^{e}R^{j}, —(CH₂)ₛNR^{e}C(O)R^{j}, —(CH₂)ₛNR^{e}C(O)OR^{j}, —(CH₂)ₛNR^{e}C(O)N(R^{e})₂, —(CH₂)ₛNR^{e}C(O)NR^{e}R^{j}, —(CH₂)ₛNR^{e}S(O)ₘR^{j}, —(CH₂)ₛNR^{e}S(O)mN(R^{e})₂, —(CH₂)ₛNR^{e}S(O)mNR^{e}R^{j}, and —(CH₂)ₛNR^{e}R^{j}, wherein each CH₂, alkyl, alkenyl, alkynyl, cycloalkyl, and cycloheteroalkyl is unsubstituted or substituted with one to five substituents selected from R^{d}, and wherein R² and R³ and the carbon atoms they are connected to can form a —C₃₋₅cycloalkyl ring.

In another embodiment of the present invention, R² is selected from the group consisting of: hydrogen, —C₁₋₆alkyl, —C₂₋₆alkenyl, —C₂₋₆alkynyl, —C₃₋₁₀cycloalkyl, —C₂₋₁₀cycloheteroalkyl, —(CH₂)ₛC(O)R^{j}, —(CH₂)ₛC(O)NR^{e}R^{j}, —(CH₂)ₛNR^{e}C(O)R^{j}, —(CH₂)ₛNR^{e}C(O)OR^{j}, —(CH₂)ₛNR^{e}C(O)N(R^{e})₂, —(CH₂)ₛNR^{e}C(O)NR^{e}R^{j}, —(CH₂)ₛNR^{e}S(O)ₘR^{j}, —(CH₂)ₛNR^{e}S(O)mN(R^{e})₂, —(CH₂)ₛNR^{e}S(O)mNR^{e}R^{j}, and —(CH₂)ₛNR^{e}R^{j}, wherein each CH₂, alkyl, alkenyl, alkynyl, cycloalkyl, and cycloheteroalkyl is unsubstituted or substituted with one to five substituents selected from R^{d}, and wherein R⁵ and R⁶ and the carbon atoms they are connected to can form a —C₃₋₅cycloalkyl ring.

In another embodiment, R² is selected from the group consisting of: hydrogen, —C₁₋₆alkyl, —C₂₋₆alkenyl, —C₂₋₆alkynyl, —C₃₋₁₀cycloalkyl, —C₂₋₁₀cycloheteroalkyl, C₁₋₆alkyl-O—C₁₋₆alkyl-, —(CH₂)ₛC(O)R^{j}, —(CH₂)ₛC(O)NR^{e}R^{j}, —(CH₂)ₛNR^{e}C(O)R^{j}, —(CH₂)ₛNR^{e}C(O)OR^{j}, —(CH₂)ₛNR^{e}C(O)N(R^{e})₂, —(CH₂)ₛNR^{e}C(O)NR^{e}R^{j}, —(CH₂)ₛNR^{e}S(O)ₘR^{j}, —(CH₂)ₛNR^{e}S(O)mN(R^{e})₂, —(CH₂)ₛNR^{e}S(O)mNR^{e}R^{j}, and —(CH₂)ₛNR^{e}R^{j}, wherein each CH₂, alkyl, alkenyl, alkynyl, cycloalkyl, and cycloheteroalkyl is unsubstituted or substituted with one to five substituents selected from R^{d}.

In another embodiment, R² is selected from the group consisting of: hydrogen, —C₁₋₆alkyl, —C₂₋₆alkenyl, —C₂₋₆alkynyl, —C₂₋₁₀cycloheteroalkyl, and —C₁₋₆alkyl-O—C₁₋₆alkyl-, wherein each alkyl, alkenyl, alkynyl, cycloalkyl, and cycloheteroalkyl is unsubstituted or substituted with one to five substituents selected from R^{d}.

In another embodiment, R² is selected from the group consisting of: hydrogen, —C₁₋₆alkyl, —C₂₋₆alkenyl, —C₂₋₆alkynyl, —C₃₋₁₀cycloalkyl, and —C₂₋₁₀cycloheteroalkyl, wherein each alkyl, alkenyl, alkynyl, cycloalkyl, and cycloheteroalkyl is unsubstituted or substituted with one to five substituents selected from R^{d}.

In another embodiment, R² is selected from the group consisting of: hydrogen, —C₁₋₆alkyl, —C₃₋₁₀cycloalkyl, and —C₂₋₁₀cycloheteroalkyl, wherein each alkyl, cycloalkyl, and cycloheteroalkyl is unsubstituted or substituted with one to five substituents selected from R^{d}.

In another embodiment, R² is selected from the group consisting of: hydrogen, and —C₁₋₆alkyl, wherein each alkyl is unsubstituted or substituted with one to five substituents selected from R^{d}. In another embodiment, R² is hydrogen, and —CH₃.

In another embodiment, R² is —C₁₋₆alkyl, wherein each alkyl is unsubstituted or substituted with one to five substituents selected from R^{d}. In another embodiment, R² is —CH₃. In another embodiment, R² is hydrogen.

In one embodiment of the present invention, R³ is selected from the group consisting of: hydrogen, —C₁₋₆alkyl, —C₂₋₆alkenyl, —C₂₋₆alkynyl, —C₃₋₁₀cycloalkyl, —C₂₋₁₀cycloheteroalkyl, —(CH₂)ₛC(O)R^{j}, —(CH₂)ₛC(O)NR^{e}R^{j}, —(CH₂)ₛNR^{e}C(O)R^{j}, —(CH₂)ₛNR^{e}C(O)ORI, —(CH₂)ₛNR^{e}C(O)N(R^{e})₂, —(CH₂)ₛNR^{e}C(O)NR^{e}R^{j}, —(CH₂)ₛNR^{e}S(O)ₘR^{j}, —(CH₂)ₛNR^{e}S(O)mN(R^{e})₂, —(CH₂)ₛNR^{e}S(O)mNR^{e}R^{j}, and —(CH₂)ₛNR^{e}R^{j}, wherein each CH₂, alkyl, alkenyl, alkynyl, cycloalkyl, and cycloheteroalkyl is unsubstituted or substituted with one to five substituents selected from R^{f}.

In another embodiment, R³ is selected from the group consisting of: hydrogen, —C₁₋₆alkyl, —C₂₋₆alkenyl, —C₂₋₆alkynyl, —C₃₋₁₀cycloalkyl, —C₂₋₁₀cycloheteroalkyl, and —C₁₋₆alkyl-O—C₁₋₆alkyl-, wherein each alkyl, alkenyl, alkynyl, cycloalkyl, and cycloheteroalkyl is unsubstituted or substituted with one to five substituents selected from R^{f}.

In another embodiment, R³ is selected from the group consisting of: hydrogen, —C₁₋₆alkyl, —C₂₋₆alkenyl, —C₂₋₆alkynyl, —C₂₋₁₀cycloheteroalkyl, wherein each CH₂, alkyl, alkenyl, alkynyl, cycloalkyl, and cycloheteroalkyl is unsubstituted or substituted with one to five substituents selected from R^{f}.

In another embodiment, R³ is selected from the group consisting of: hydrogen, —C₁₋₆alkyl, —C₃₋₁₀cycloalkyl, and —C₂₋₁₀cycloheteroalkyl, wherein each alkyl, cycloalkyl, and cycloheteroalkyl is unsubstituted or substituted with one to five substituents selected from R^{f}.

In another embodiment, R³ is selected from the group consisting of: hydrogen, and —C₁₋₆alkyl, wherein each alkyl is unsubstituted or substituted with one to five substituents selected from R^{f}.

In another embodiment, R³ is selected from the group consisting of: hydrogen, and —CH₃.

In another embodiment, R³ is selected from the group consisting of: —C₁₋₆alkyl, wherein each CH₂, alkyl, alkenyl, alkynyl, cycloalkyl, and cycloheteroalkyl is unsubstituted or substituted with one to five substituents selected from R^{d}. In another embodiment, R³ is —CH₃. In another embodiment, R³ is hydrogen.

In one embodiment of the present invention, R⁴ is selected from the group consisting of: hydrogen, —C₁₋₆alkyl, —C₂₋₆alkenyl, —C₂₋₆alkynyl, —C₃₋₁₀cycloalkyl, —C₂₋₁₀cycloheteroalkyl, —C₁₋₆alkyl-O—C₁₋₆alkyl-, —(CH₂)ₛC(O)R^{j}, —(CH₂)ₛC(O)NR^{e}R^{j}, —(CH₂)ₛNR^{e}C(O)R^{j}, —(CH₂)ₛNR^{e}C(O)ORI, —(CH₂)ₛNR^{e}C(O)N(R^{e})₂, —(CH₂)ₛNR^{e}C(O)NR^{e}R^{j}, —(CH₂)ₛNR^{e}S(O)ₘR^{j}, —(CH₂)ₛNR^{e}S(O)mN(R^{e})₂, —(CH₂)ₛNR^{e}S(O)mNR^{e}R^{j}, and —(CH₂)ₛNR^{e}R^{j}, wherein each CH₂, alkyl, alkenyl, alkynyl, cycloalkyl, and cycloheteroalkyl is unsubstituted or substituted with one to five substituents selected from R^{g}.

In another embodiment of the present invention, R⁴ is selected from the group consisting of: hydrogen, —C₁₋₆alkyl, —C₂₋₆alkenyl, —C₂₋₆alkynyl, —C₃₋₁₀cycloalkyl, —C₂₋₁₀cycloheteroalkyl, and —C₁₋₆alkyl-O—C₁₋₆alkyl-, wherein each CH₂, alkyl, alkenyl, alkynyl, cycloalkyl, and cycloheteroalkyl is unsubstituted or substituted with one to five substituents selected from R^{g}.

In another embodiment, R⁴ is selected from the group consisting of: hydrogen, —C₁₋₆alkyl, —C₃₋₁₀cycloalkyl, and —C₂₋₁₀cycloheteroalkyl, wherein each alkyl, cycloalkyl, and cycloheteroalkyl is unsubstituted or substituted with one to five substituents selected from R^{g}. In another embodiment, R⁴ is selected from the group consisting of: hydrogen, and —C₁₋₆alkyl, wherein each alkyl is unsubstituted or substituted with one to five substituents selected from R^{g}. In another embodiment, R⁴ is selected from the group consisting of: hydrogen, and —CH₃.

In another embodiment, R⁴ is —C₁₋₆alkyl, wherein each alkyl is unsubstituted or substituted with one to five substituents selected from R^{g}. In another embodiment of the present invention, R⁴ is —CH₃. In another embodiment, R⁴ is hydrogen.

In one embodiment of the present invention, R⁵ is selected from the group consisting of: hydrogen, —C₁₋₆alkyl, —C₂₋₆alkenyl, —C₂₋₆alkynyl, —C₃₋₁₀cycloalkyl, —C₂₋₁₀cycloheteroalkyl, —C₁₋₆alkyl-O—C₁₋₆alkyl-, —(CH₂)ₛC(O)R^{j}, —(CH₂)ₛC(O)NR^{e}R^{j}, —(CH₂)ₛNR^{e}C(O)R^{j}, —(CH₂)ₛNR^{e}C(O)OR^{j}, —(CH₂)ₛNR^{e}C(O)N(R^{e})₂, —(CH₂)ₛNR^{e}C(O)NR^{e}R^{j}, —(CH₂)ₛNR^{e}S(O)ₘR^{j}, —(CH₂)ₛNR^{e}S(O)mN(R^{e})₂, —(CH₂)ₛNR^{e}S(O)mNR^{e}R^{j}, and —(CH₂)ₛNR^{e}R^{j}, wherein each CH₂, alkyl, alkenyl, alkynyl, cycloalkyl, and cycloheteroalkyl is unsubstituted or substituted with one to five substituents selected from R^{g}, and R⁵ and R⁴ and the carbon atoms are connected to can form a —C₃₋₅cycloalkyl

In another embodiment of the present invention, R⁵ is selected from the group consisting of: hydrogen, —C₁₋₆alkyl, —C₂₋₆alkenyl, —C₂₋₆alkynyl, —C₃₋₁₀cycloalkyl, —C₂₋₁₀ cycloheteroalkyl, —C₁₋₆alkyl-O—C₁₋₆alkyl-, —(CH₂)ₛC(O)R^{j}, —(CH₂)ₛC(O)NR^{e}R^{j}, —(CH₂)ₛNR^{e}C(O)R^{j}, —(CH₂)ₛNR^{e}C(O)OR^{j}, —(CH₂)ₛNR^{e}C(O)N(R^{e})₂, —(CH₂)ₛNR^{e}C(O)NR^{e}R^{j}, —(CH₂)ₛNR^{e}S(O)ₘR^{j}, —(CH₂)ₛNR^{e}S(O)mN(R^{e})₂, —(CH₂)ₛNR^{e}S(O)mNR^{e}R^{j}, and —(CH₂)ₛNR^{e}R^{j}, wherein each CH₂, alkyl, alkenyl, alkynyl, cycloalkyl, and cycloheteroalkyl is unsubstituted or substituted with one to five substituents selected from R^{g}.

In another embodiment, R⁵ is selected from the group consisting of: hydrogen, —C₁₋₆alkyl, —C₂₋₆alkenyl, —C₂₋₆alkynyl, —C₃₋₁₀cycloalkyl, —C₂₋₁₀cycloheteroalkyl, —C₁₋₆alkyl-O—C₁₋₆alkyl-, wherein each CH₂, alkyl, alkenyl, alkynyl, cycloalkyl, and cycloheteroalkyl is unsubstituted or substituted with one to five substituents selected from R^{g}. In another embodiment, R⁵ is selected from the group consisting of: hydrogen, —C₁₋₆alkyl, —C₃₋₁₀cycloalkyl, and —C₂₋₁₀cycloheteroalkyl, wherein each alkyl, cycloalkyl, and cycloheteroalkyl is unsubstituted or substituted with one to five substituents selected from R^{g}.

In another embodiment, R⁵ is selected from the group consisting of: hydrogen, and —C₁₋₆alkyl, wherein each alkyl is unsubstituted or substituted with one to five substituents selected from R^{g}. In another embodiment, R⁵ is selected from the group consisting of: hydrogen, and —CH₃. In another embodiment, R⁵ is —C₁₋₆alkyl, wherein each alkyl is unsubstituted or substituted with one to five substituents selected from R^{g}. In another embodiment, R⁵ is —CH₃. In another embodiment, R⁵ is hydrogen.

In one embodiment of the present invention, R⁶ is selected from the group consisting of: hydrogen, and —C₁₋₆alkyl, wherein each alkyl is unsubstituted or substituted with one to five halogen substituents. In another embodiment, R⁶ is selected from the group consisting of: hydrogen, and —CH₃. In another embodiment, R⁶ is —C₁₋₆alkyl, wherein each alkyl is unsubstituted or substituted with one to five halogen substituents. In another embodiment of the present invention, R⁶ is —CH₃. In another embodiment of the present invention, R⁶ is hydrogen. In one embodiment of the present invention, R⁷ is selected from the group consisting of:

hydrogen, —C₁₋₆alkyl, —C₃₋₆cycloalkyl, and —C₂₋₆cycloheteroalkyl, wherein each alkyl, cycloalkyl and cycloheteroalkyl is unsubstituted or substituted with one to five halogen substituents. In another embodiment of the present invention, R⁷ is selected from the group consisting of: hydrogen, and —C₁₋₆alkyl, wherein each alkyl is unsubstituted or substituted with one to five halogen substituents. In another embodiment of the present invention, R⁷ is —C₁₋₆alkyl, wherein each alkyl is unsubstituted or substituted with one to five halogen substituents. In another embodiment of the present invention, R⁷ is hydrogen.

In one embodiment of the present invention, R⁸ is selected from the group consisting of: hydrogen, —C₁₋₆alkyl, —C₂₋₆alkenyl, and —C₂₋₆alkynyl, wherein each alkyl, alkenyl and alkynyl is unsubstituted or substituted with one to five halogen substituents. In another embodiment of the present invention, R⁸ is selected from the group consisting of: hydrogen, and —C₁₋₆alkyl, wherein each alkyl is unsubstituted or substituted with one to five halogen substituents. In another embodiment of the present invention, R⁸ is —C₁₋₆alkyl, wherein each alkyl is unsubstituted or substituted with one to five halogen substituents. In another embodiment of the present invention, R⁸ is hydrogen.

In another embodiment of the present invention, each R^{a} is independently selected from the group consisting of: —CF₃, —OCF₃, —CHF₂, —OCHF₂, —CH₂CF₃, —OCH₂CF₃, —CF₂CH₃, CN, oxo, halogen, —S(O)₂C₁₋₆alkyl, —C₁₋₆alkyl, —C₂₋₆alkenyl, —C₂₋₆alkynyl, —O—C₁₋₆alkyl, —C₃₋₆cycloalkyl, —O—C₃₋₆cycloalkyl, —C₂₋₆cycloheteroalkyl, aryl, heteroaryl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —C₂₋₆alkenyl-C₃₋₆cycloalkyl, —C₂₋₆alkenyl-C₂₋₆cycloheteroalkyl, —C₂₋₆alkenyl-aryl, —C₂₋₆alkenyl-heteroaryl, —C₂₋₆alkynyl-C₃₋₆cycloalkyl, —C₂₋₆alkynyl-C₂₋₆cycloheteroalkyl, —C₂₋₆alkynyl-aryl, —C₂₋₆alkynyl-heteroaryl, —OH, —(CH₂)ₚ—OC₁₋₆alkyl, —(CH₂)ₚ—OC₂₋₆alkenyl, —(CH₂)ₚ—OC₂₋₆alkynyl, —(CH₂)ₚ—OC₃₋₆cycloalkyl, —(CH₂)ₚ—OC₂₋₆heterocycloalkyl, —(CH₂)ₚ—O-aryl, —(CH₂)ₚ—O-heteroaryl, —OC₁₋₆alkyl-C₃₋₆cycloalkyl, —OC₁₋₆alkyl-C₂₋₆heterocycloalkyl, —OC₁₋₆alkyl-aryl, —OC₁₋₆alkyl-heteroaryl, —S(O)ₘR^{j}, —N(R^{k})₂, and —NR^{k}R^{L}, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group consisting of: —CF₃, —OCF₃, —CHF₂, —OCHF₂, —CH₂CF₃, —OCH₂CF₃, —CF₂CH₃, CN, oxo, halogen, —S(O)₂C₁₋₆alkyl, —C₁₋₆alkyl, —C₂₋₆alkenyl, —C₂₋₆alkynyl, —O—C₁₋₆alkyl, —C₃₋₆cycloalkyl, —O—C₃₋₆cycloalkyl, —C₂₋₆cycloheteroalkyl, aryl, heteroaryl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —C₂₋₆alkenyl-C₃₋₆cycloalkyl, —C₂₋₆alkenyl-C₂₋₆cycloheteroalkyl, —C₂₋₆alkenyl-aryl, —C₂₋₆alkenyl-heteroaryl, —C₂₋₆alkynyl-C₃₋₆cycloalkyl, —C₂₋₆alkynyl-C₂₋₆cycloheteroalkyl, —C₂₋₆alkynyl-aryl, —C₂₋₆alkynyl-heteroaryl, and —OH, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group consisting of: —CF₃, —OCF₃, —CHF₂, —OCHF₂, —CH₂CF₃, —OCH₂CF₃, —CF₂CH₃, CN, oxo, halogen, —S(O)₂C₁₋₆alkyl, —C₁₋₆alkyl, —C₂₋₆alkenyl, —C₂₋₆alkynyl, —C₃₋₆cycloalkyl, —O—C₃₋₆cycloalkyl, —C₂₋₆cycloheteroalkyl, aryl, heteroaryl, —C₁₋₆alkyl-aryl, —C₁₋₆alkyl-heteroaryl, —C₁₋₆alkyl-C₃₋₆cycloalkyl, and —C₁₋₆alkyl-C₂₋₆cycloheteroalkyl, wherein each R^{a} is unsubstituted or substituted with one to six substituents selected from halogen, CF₃, OH, C₁₋₆alkyl, and —OC₁₋₆alkyl.

In another embodiment, each R^{a} is independently selected from the group consisting of: —CF₃, —OCF₃, —CHF₂, —OCHF₂, —CH₂CF₃, —OCH₂CF₃, —CF₂CH₃, CN, oxo, halogen, —S(O)₂C₁₋₆alkyl, —C₁₋₆alkyl, —C₂₋₆alkenyl, —C₂₋₆alkynyl, and —O—C₁₋₆alkyl, wherein each R^{a} is unsubstituted or substituted with one to six substituents selected from halogen, CF₃, OH, C₁₋₆alkyl, and OC₁₋₆alkyl.

In another embodiment of the present invention, each R^{a} is independently selected from the group consisting of: —CF₃, —OCF₃, —CHF₂, —OCHF₂, —CH₂CF₃, —OCH₂CF₃, —CF₂CH₃, CN, oxo, halogen, —S(O)₂C₁₋₆alkyl, —C₁₋₆alkyl, —C₂₋₆alkenyl, —C₂₋₆alkynyl, and —O—C₁₋₆alkyl, wherein each R^{a} is unsubstituted or substituted with one to six substituents selected from halogen, CF₃, OH, C₁₋₆alkyl, and OC₁₋₆alkyl.

In another embodiment of the present invention, each R^{a} is independently selected from the group consisting of: —CF₃, —OCF₃, —CHF₂, —OCHF₂, —CH₂CF₃, —OCH₂CF₃, —CF₂CH₃, CN, oxo, halogen, —S(O)₂C₁₋₆alkyl, and —C₁₋₆alkyl, wherein each R^{a} is unsubstituted or substituted with one to six substituents selected from halogen, CF₃, OH, C₁₋₆alkyl, and OC₁₋₆alkyl.

In another embodiment of the present invention, each R^{a} is independently selected from the group consisting of: —CF₃, —OCF₃, —CHF₂, —OCHF₂, —CH₂CF₃, —OCH₂CF₃, —CF₂CH₃, CN, halogen, and —C₁₋₆alkyl, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group consisting of: —CF₃, —OCF₃, and halogen. In another embodiment, each R^{a} is independently selected from the group consisting of: —CF₃, —OCF₃, F, and C₁.

In another embodiment of the present invention, each R^{a} is independently selected from the group consisting of: —CF₃, and halogen. In another embodiment, each R^{a} is independently selected from the group consisting of: —CF₃, F, and C₁.

In another embodiment of the present invention, each R^{b} is independently selected from the group consisting of: —CF₃, —OCF₃, —OCHF₂, —CH₂CF₃, —OCH₂CF₃, —CF₂CH₃, CN, oxo, halogen, —S(O)₂C₁₋₆alkyl, —C₂₋₆alkenyl, —C₂₋₆alkynyl, —C₃₋₆cycloalkyl, —O—C₃₋₆cycloalkyl, —C₂₋₆cycloheteroalkyl, aryl, heteroaryl, —C₁₋₆alkyl-heteroaryl, —C₁₋₆alkyl-C₃₋₆cycloalkyl, —C₁₋₆alkyl-C₂₋₆cycloheteroalkyl, 6alkenyl-C₃₋₆cycloalkyl, —C₂₋₆alkenyl-C₂₋₆cycloheteroalkyl, —C₂₋₆alkenyl-aryl, —C₂₋₆alkenyl-heteroaryl, —C₂₋₆alkynyl-C₃₋₆cycloalkyl, —C₂₋₆alkynyl-C₂₋₆cycloheteroalkyl, —C₂₋₆alkynyl-aryl, —C₂₋₆alkynyl-heteroaryl, —OH, —(CH₂)_{q}—OC₁₋₆alkyl, —(CH₂)_{q}—OC₂₋₆alkenyl, —(CH₂)_{q}—OC₂₋₆alkynyl, —(CH₂)_{q}—OC₃₋₆cycloalkyl, —(CH₂)_{q}—OC₂₋₆heterocycloalkyl, —(CH₂)_{q}—O-aryl, —(CH₂)_{q}—O-heteroaryl, —C₁₋₆alkyl-C₃₋₆cycloalkyl, —C₁₋₆alkyl-C₂₋₆heterocycloalkyl, —OC₁₋₆alkyl-aryl, —OC₁₋₆alkyl-heteroaryl, —S(O)ₘRⁱ, —C₁₋₆alkyl-S(O)ₘRⁱ, —C(O)R^{L}, and —NR^{k}R^{L}, wherein each R^{b} is unsubstituted or substituted with one to six substituents selected from halogen, CF₃, OCF₃, CN, CH₂CF₃, CF₂CH₃, C₁₋₆alkyl, and —OC₁₋₆alkyl.

In another embodiment, each R^{b} is independently selected from the group consisting of: —CF₃, —OCF₃, —OCHF₂, —CH₂CF₃, —OCH₂CF₃, —CF₂CH₃, CN, oxo, halogen, —S(O)₂C₁₋₆alkyl, —C₁₋₆alkyl, —C₂₋₆alkenyl, —C₂₋₆alkynyl, —C₃₋₆cycloalkyl, C₃₋₆cycloalkyl, —C₂₋₆cycloheteroalkyl, aryl, heteroaryl, —C₁₋₆alkyl-heteroaryl, —C₁₋₆alkyl-C₃₋₆cycloalkyl, —C₁₋₆alkyl-C₂₋₆cycloheteroalkyl, —C₂₋₆alkenyl-C₃₋₆cycloalkyl, —C₂₋₆alkenyl-C₂₋₆cycloheteroalkyl, —C₂₋₆alkenyl-aryl, —C₂₋₆alkenyl-heteroaryl, —C₂₋₆alkynyl-C₃₋₆cycloalkyl, —C₂₋₆alkynyl-C₂₋₆cycloheteroalkyl, —C₂₋₆alkynyl-aryl, —C₂₋₆alkynyl-heteroaryl, and —OH, wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl.

In another embodiment, each R^{b} is independently selected from the group consisting of: —CF₃, —OCF₃, —CHF₂, —OCHF₂, —CH₂CF₃, —OCH₂CF₃, —CF₂CH₃, CN, oxo, halogen, —S(O)₂C₁₋₆alkyl, —C₁₋₆alkyl, —C₂₋₆alkenyl, —C₂₋₆alkynyl, —C₃₋₆cycloalkyl, —C₂₋₆cycloheteroalkyl, aryl, and heteroaryl, wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl.

In another embodiment, each R^{b} is independently selected from the group consisting of: —CF₃, —OCF₃, —OCHF₂, —CH₂CF₃, —OCH₂CF₃, —CF₂CH₃, CN, oxo, halogen, —S(O)₂C₁₋₆alkyl, —C₁₋₆alkyl, —C₂₋₆alkenyl, —C₂₋₆alkynyl, —C₁₋₆alkyl, —C₃₋₆cycloalkyl, and —C₂₋₆cycloheteroalkyl, wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl.

In another embodiment, each R^{b} is independently selected from the group consisting of: —CF₃, —OCF₃, —OCHF₂, —CH₂CF₃, —OCH₂CF₃, —CF₂CH₃, CN, halogen, —C₁₋₆alkyl, —C₂₋₆alkenyl, —C₂₋₆alkynyl, —C₃₋₆cycloalkyl, and —C₂₋₆cycloheteroalkyl, wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl.

In another embodiment, each R^{b} is independently selected from the group consisting of: —CF₃, —OCF₃, —OCHF₂, —CH₂CF₃, —OCH₂CF₃, —CF₂CH₃, CN, halogen, —C₁₋₆alkyl, —O—C₁₋₆alkyl, and —C₃₋₆cycloalkyl, wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl.

In another embodiment, each R^{b} is independently selected from the group consisting of: —CF₃, —OCF₃, —OCHF₂, CN, halogen, —C₁₋₆alkyl, —O—C₁₋₆alkyl, and —C₃₋₆cycloalkyl, wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl.

In another embodiment, each R^{b} is independently selected from the group consisting of: —CF₃, —OCF₃, —OCHF₂, CN, F, C₁, —CH₃, —CH(CH₃)₂, —OCH₃, and cyclopropyl, wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl.

In another embodiment, each R^{b} is independently selected from the group consisting of: —CF₃, and halogen. In another embodiment, each R^{b} is independently selected from the group consisting of: —CF₃, F, and Cl.

In one embodiment of the present invention, R^{c} is selected from: —C₁₋₆alkyl, OH, halogen, and —OC₁₋₆alkyl, wherein alkyl can be unsubstituted or substituted with one to three halogens. In another embodiment, R^{c} is selected from: —C₁₋₆alkyl, OH, and halogen, wherein alkyl can be unsubstituted or substituted with one to three halogens. In another embodiment, R^{c} is selected from: OH, and halogen. In a class of this embodiment, R^{c} is selected from: OH, and F. In another embodiment, R^{c} is OH. In another embodiment, R^{c} is halogen. In a class of this embodiment, R^{c} is F.

In one embodiment of the present invention, R^{d} is selected from: —C₁₋₆alkyl, OH, halogen, and —OC₁₋₆alkyl, wherein alkyl can be unsubstituted or substituted with one to three halogens. In another embodiment, R^{d} is selected from: —C₁₋₆alkyl, OH, and halogen, wherein alkyl can be unsubstituted or substituted with one to three halogens. In another embodiment, R^{d} is selected from: OH, and halogen. In a class of this embodiment, R^{d} is selected from: OH, and F. In another embodiment, R^{d} is OH. In another embodiment, R^{d} is halogen. In a class of this embodiment, R^{d} is F.

In one embodiment of the present invention, R^{e} is selected from: hydrogen and C₁₋₆alkyl. In another embodiment, R^{e} is hydrogen. In another embodiment, R^{e} is C₁₋₆alkyl.

In one embodiment of the present invention, R^{f} is selected from: —C₁₋₆alkyl, OH, halogen, and —OC₁₋₆alkyl, wherein alkyl can be unsubstituted or substituted with one to three halogens. In another embodiment, R^{f} is selected from: —C₁₋₆alkyl, OH, and halogen, wherein alkyl can be unsubstituted or substituted with one to three halogens. In another embodiment, R^{f} is selected from: OH, and halogen. In a class of this embodiment, R^{f} is selected from: OH, and F. In another embodiment, R^{f} is OH. In another embodiment, R^{f} is halogen. In a class of this embodiment, R^{f} is F.

In one embodiment of the present invention, R^{g} is selected from: —C₁₋₆alkyl, OH, halogen, and —OC₁₋₆alkyl, wherein alkyl can be unsubstituted or substituted with one to three halogens. In another embodiment, R^{g} is selected from: —C₁₋₆alkyl, OH, and halogen, wherein alkyl can be unsubstituted or substituted with one to three halogens. In another embodiment, R^{g} is selected from: OH, and halogen. In a class of this embodiment, R^{g} is selected from: OH, and F. In another embodiment, R^{g} is OH. In another embodiment, R^{g} is halogen. In a class of this embodiment, R^{g} is F.

In one embodiment of the present invention, R^{h} is selected from: hydrogen and C₁₋₆alkyl. In another embodiment, R^{h} is hydrogen. In another embodiment, R^{h} is C₁₋₆alkyl.

In one embodiment of the present invention, Rⁱ is selected from: hydrogen, C₁₋₆alkyl, C₃₋₆cycloalkyl, aryl, and heteroaryl. In another embodiment, Rⁱ is selected from: hydrogen, C₁₋₆alkyl, and C₃₋₆cycloalkyl. In another embodiment, Rⁱ is selected from: hydrogen and C₁₋₆alkyl. In another embodiment, Rⁱ is hydrogen. In another embodiment, Rⁱ is C₁₋₆alkyl. In one embodiment of the present invention, R^{j} is selected from: hydrogen, C₁₋₆alkyl, C₃₋₆alkenyl, C₃₋₆alkynyl, C₃₋₆cycloalkyl, C₂₋₅cycloheteroalkyl, aryl, and heteroaryl. In another embodiment, R^{j} is selected from: hydrogen, C₁₋₆alkyl, C₃₋₆alkenyl, C₃₋₆alkynyl, C₃₋₆cycloalkyl, and C₂₋₅cycloheteroalkyl. In another embodiment, R^{j} is selected from: hydrogen, C₁₋₆alkyl, C₃₋₆alkenyl, C₃₋₆alkynyl, and C₃₋₆cycloalkyl. In another embodiment, R^{j} is selected from: hydrogen, C₁₋₆alkyl, C₃₋₆alkenyl, and C₃₋₆alkynyl. In another embodiment, R^{j} is selected from: hydrogen, C₁₋₆alkyl, and C₃₋₆alkenyl. In another embodiment, R^{j} is selected from: hydrogen, and C₁₋₆alkyl. In another embodiment, R^{j} is C₁₋₆alkyl. In another embodiment, R^{j} is hydrogen.

In one embodiment of the present invention, R^{k} is selected from: hydrogen and C₁₋₆alkyl. In another embodiment, R^{k} is hydrogen. In another embodiment, R^{k} is C₁₋₆alkyl.

In one embodiment of the present invention, R^{L} is selected from: hydrogen, C₁₋₆alkyl, C₃₋₆cycloalkyl, aryl, and heteroaryl. In another embodiment, R^{L} is selected from: hydrogen, C₁₋₆alkyl, and C₃₋₆cycloalkyl. In another embodiment, R^{L} is selected from: hydrogen, and C₁₋₆alkyl. In another embodiment, R^{L} is hydrogen. In another embodiment, R^{L} is C₁₋₆alkyl.

In one embodiment of the present invention, m is 0, 1 or 2. In another embodiment, m is 0 or 1. In another embodiment, m is 0 or 2. In another embodiment, m is 0. In another embodiment, m is 1. In another embodiment, m is 2.

In one embodiment of the present invention, n is 2, 3, 4, 5 or 6. In another embodiment, n is 2, 3, 4, or 5. In another embodiment, n is 2, 3, or 4. In another embodiment, n is 2 or 3. In another embodiment, n is 2 or 4. In another embodiment, n is 2, 3, 4, or 5. In another embodiment, n is 3. In another embodiment, n is 4. In another embodiment, n is 5. In another embodiment, n is 6.

In one embodiment of the present invention, p is 0, 1, 2 or 3. In another embodiment, p is 0, 1 or 2. In another embodiment, p is 0, 1 or 3. In another embodiment, p is 1, 2 or 3. In another embodiment, p is 1 or 2. In another embodiment, p is 1 or 3. In another embodiment, p is 0 or 1. In another embodiment, p is 0 or 2. In another embodiment, p is 0 or 3. In another embodiment, p is 0. In another embodiment, p is 1. In another embodiment, p is 2. In another embodiment, p is 3.

In one embodiment of the present invention, q is 0, 1, 2 or 3. In another embodiment, q is 1, 2 or 3. In another embodiment, q is 0, 1 or 2. In another embodiment, q is 0, 1 or 3. In another embodiment, q is 0, or 1. In another embodiment, q is 0 or 2. In another embodiment, q is 0. In another embodiment, q is 1. In another embodiment, q is 2. In another embodiment, q is 3.

In one embodiment of the present invention, r is 0, 1 or 2. In another embodiment, r is 1 or 2. In another embodiment, r is 0 or 1. In another embodiment, r is 0 or 2. In another embodiment, r is 0. In another embodiment, r is 1. In another embodiment, r is 2.

In one embodiment of the present invention, s is 0, 1, 2, 3, 4, 5 or 6. In another embodiment, s is 0, 1, 2, 3, 4, or 5. In another embodiment, s is 1, 2, 3, 4, 5 or 6. In another embodiment, s is 1, 2, 3, 4 or 5. In another embodiment, s is 0, 1, 2, 3, or 4. In another embodiment, s is 1, 2, 3, or 4. In another embodiment, s is 0, 1, 2, or 3. In another embodiment, s is 1, 2, or 3. In another embodiment, s is 0, 1 or 2. In another embodiment, s is 1 or 2. In another embodiment, s is 0. In another embodiment, s is 1. In another embodiment, s is 2. In another embodiment, s is 3. In another embodiment, s is 4. In another embodiment, s is 5. In another embodiment, s is 6.

In another embodiment of the present invention, the invention relates to compounds of structural formula Ia:

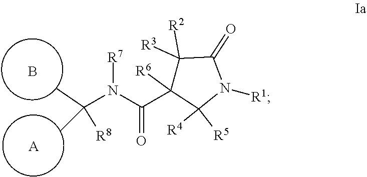

wherein A is aryl; or a pharmaceutically acceptable salt thereof.

In another embodiment of the present invention, the invention relates to compounds of structural formula Ib:

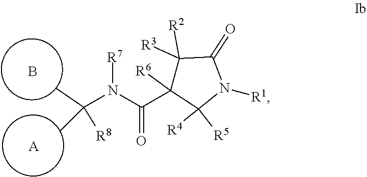

wherein A is heteroaryl; or a pharmaceutically acceptable salt thereof.

In another embodiment of the present invention, the invention relates to compounds of structural formula Ic:

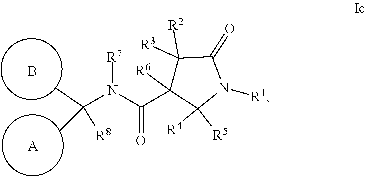

wherein A is phenyl; or a pharmaceutically acceptable salt thereof.

In another embodiment of the present invention, the invention relates to compounds of structural formula Id:

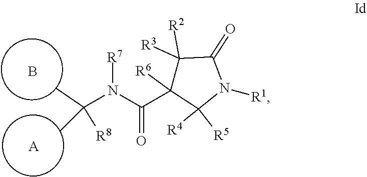

wherein A is pyridine; or a pharmaceutically acceptable salt thereof.

In another embodiment of the present invention, the invention relates to compounds of structural formula Ie:

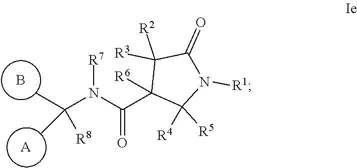

wherein B is aryl; or a pharmaceutically acceptable salt thereof.

In another embodiment of the present invention, the invention relates to compounds of structural formula If:

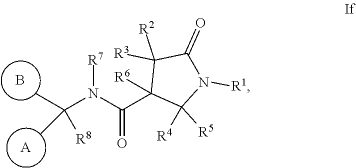

wherein B is heteroaryl; or a pharmaceutically acceptable salt thereof.

In another embodiment of the present invention, the invention relates to compounds of structural formula Ig:

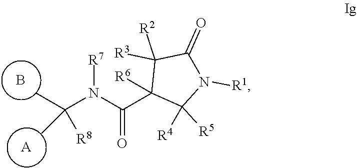

wherein B is phenyl; or a pharmaceutically acceptable salt thereof.

In another embodiment of the present invention, the invention relates to compounds of structural formula Ih:

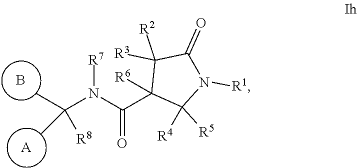

wherein B is pyridine; or a pharmaceutically acceptable salt thereof.

The compound of structural formula I, includes the compounds of structural formulas Ia, Ib, Ic, Id, Ie, If, Ig, and Ih, and pharmaceutically acceptable salts, hydrates and solvates thereof.

Another embodiment of the present invention relates to compounds of structural formula I wherein:

A is selected from the group consisting of:
1) aryl, and
2) heteroaryl,
   wherein aryl and heteroaryl are unsubstituted or substituted with one to five substituents selected from R^{a};
   B is independently selected from the group consisting of:
1) aryl,
2) heteroaryl,
3) —C₁₋₆alkyl-aryl,
4) —C₃₋₈cycloalkyl-aryl,
5) —C₂₋₈cycloheteroalkyl-aryl,
6) —C₁₋₆alkyl-heteroaryl,
7) —C₃₋₈cycloalkyl-heteroaryl,
8) —C₂₋₈cycloheteroalkyl-heteroaryl,
9) —C₁₋₆alkyl-O-aryl,
10) C₁₋₆alkyl-O-heteroaryl,
11) —C₃₋₁₂cycloalkyl,
12) —C₂₋₁₂cycloheteroalkyl,
13) C₁₋₆alkyl-C₃₋₁₂cycloalkyl,
14) —C₁₋₆alkyl-C₂₋₁₂cycloheteroalkyl,
15) —C₁₋₆alkyl-O—C₃₋₁₂cycloalkyl,
16) —C₁₋₆alkyl-O—C₂₋₁₂cycloheteroalkyl,
17) —C₀₋₆alkyl-aryl fused to C₄₋₆cycloalkyl or C₄₋₆cycloheteroalkyl containing 1-3 heteroatoms independently selected from O, S and N(R^{h})₂,
18) —C₀₋₆alkyl-aryl fused to C₄₋₆cycloalkenyl or C₄₋₆cycloheteroalkenyl containing 1-3 heteroatoms independently selected from O, S and N(R^{h})₂,
19) —C₀₋₆alkyl-heteroaryl fused to C₄₋₆cycloalkyl or C₄₋₆cycloheteroalkyl containing 1-3 heteroatoms independently selected from O, S and N(R^{h})₂, and
20) —C₀₋₆alkyl-heteroaryl fused to C₄₋₆cycloalkenyl or C₄₋₆cycloheteroalkenyl containing 1-3 heteroatoms independently selected from O, S and N(R^{h})₂,
   wherein alkyl, cycloalkyl, cycloheteroalkyl, cycloalkenyl, aryl and heteroaryl are unsubstituted or substituted with one to five substituents selected from R^{b}; R¹ is selected from the group consisting of:
1) hydrogen,
2) —C₁₋₆alkyl,
3) —C₃₋₆alkenyl,
4) —C₃₋₆alkynyl,
5) —C₃₋₁₀cycloalkyl,
6) —C₂₋₁₀cycloheteroalkyl,
7) —C₁₋₆alkyl-O—C₁₋₆alkyl-,
8) —(CH₂)ₛC(O)R^{j},
9) —(CH₂)ₛC(O)NR^{e}R^{j},
10) —(CH₂)ₙNR^{e}C(O)R^{j},
11) —(CH₂)ₙNR^{e}C(O)OR^{j},
12) —(CH₂)ₙNR^{e}C(O)N(R^{e})₂,
13) —(CH₂)ₙNR^{e}C(O)NR^{e}R^{j},
14) —(CH₂)ₙNR^{e}S(O)ₘR^{j},
15) —(CH₂)ₙNR^{e}S(O)mN(R^{e})₂,
16) —(CH₂)ₙNR^{e}S(O)mNR^{e}R^{j}, and
17) —(CH₂)ₙNR^{e}R^{j},
   wherein each CH₂, alkyl, alkenyl, alkynyl, cycloalkyl and cycloheteroalkyl is unsubstituted or substituted with one to five substituents selected from R^{c};
   R² is selected from the group consisting of:
1) hydrogen,
2) —C₁₋₆alkyl,
3) —C₂₋₆alkenyl,
4) —C₂₋₆alkynyl,
5) —C₃₋₁₀cycloalkyl,
6) —C₂₋₁₀cycloheteroalkyl,
7) —C₁₋₆alkyl-O—C₁₋₆alkyl-,
8) —(CH₂)ₛC(O)R^{j},
9) —(CH₂)ₛC(O)NR^{e}R^{j},
10) —(CH₂)ₛNR^{e}C(O)R^{j},
11) —(CH₂)ₛNR^{e}C(O)OR^{j},
12) —(CH₂)ₛNR^{e}C(O)N(R^{e})₂,
13) —(CH₂)ₛNR^{e}C(O)NR^{e}R^{j},
14) —(CH₂)ₛNR^{e}S(O)ₘR^{j},
15) —(CH₂)ₛNR^{e}S(O)mN(R^{e})₂,
16) —(CH₂)ₛNR^{e}S(O)mNR^{e}R^{j}, and
17) —(CH₂)ₛNR^{e}R^{j},
   wherein each CH₂, alkyl, alkenyl, alkynyl, cycloalkyl, and cycloheteroalkyl is unsubstituted or substituted with one to five substituents selected from R^{d}, and wherein R² and R³ and the carbon atom they are connected to can form a —C₃₋₅cycloalkyl ring;
   R³ is selected from the group consisting of:
1) hydrogen,
2) —C₁₋₆alkyl,
3) —C₂₋₆alkenyl,
4) —C₂₋₆alkynyl,
6) —C₂₋₁₀cycloheteroalkyl,
7) —C₁₋₆alkyl-O—C₁₋₆alkyl-,
8) —(CH₂)ₛC(O)R^{j},
9) —(CH₂)ₛC(O)NR^{e}R^{j},
10) —(CH₂)ₛNR^{e}C(O)R^{j},
11) —(CH₂)ₛNR^{e}C(O)OR^{j},
12) —(CH₂)ₛNR^{e}C(O)N(R^{e})₂,
13) —(CH₂)ₛNR^{e}C(O)NR^{e}R^{j},
14) —(CH₂)ₛNR^{e}S(O)ₘR^{j},
15) —(CH₂)ₛNR^{e}S(O)mN(R^{e})₂,
16) —(CH₂)ₛNR^{e}S(O)mNR^{e}R^{j}, and
17) —(CH₂)ₛNR^{e}R^{j},
   wherein each CH₂, alkyl, alkenyl, alkynyl, cycloalkyl, and cycloheteroalkyl is unsubstituted or substituted with one to five substituents selected from R^{d};
   R⁴ is selected from the group consisting of:
1) hydrogen,
2) —C₁₋₆alkyl,
3) —C₂₋₆alkenyl,
4) —C₂₋₆alkynyl,
5) —C₃₋₁₀cycloalkyl,
6) —C₂₋₁₀cycloheteroalkyl,
7) —C₁₋₆alkyl-O—C₁₋₆alkyl-,
8) —(CH₂)ₛC(O)R^{j},
9) —(CH₂)ₛC(O)NR^{e}R^{j},
10) —(CH₂)ₛNR^{e}C(O)R^{j},
11) —(CH₂)ₛNR^{e}C(O)OR^{j},
12) —(CH₂)ₛNR^{e}C(O)N(R^{e})₂,
13) —(CH₂)ₛNR^{e}C(O)NR^{e}R^{j},
14) —(CH₂)ₛNR^{e}S(O)ₘR^{j},
15) —(CH₂)ₛNR^{e}S(O)mN(R^{e})₂,
16) —(CH₂)ₛNR^{e}S(O)mNR^{e}R^{j}, and
17) —(CH₂)ₛNR^{e}R^{j},
   wherein each CH₂, alkyl, alkenyl, alkynyl, cycloalkyl, and cycloheteroalkyl is unsubstituted or substituted with one to five substituents selected from R^{g};
   R⁵ is selected from the group consisting of:
1) hydrogen,
2) —C₁₋₆alkyl,
3) —C₂₋₆alkenyl,
4) —C₂₋₆alkynyl,
5) —C₃₋₁₀cycloalkyl,
6) —C₂₋₁₀cycloheteroalkyl,
7) —C₁₋₆alkyl-O—C₁₋₆alkyl-,
8) —(CH₂)ₛC(O)R^{j},
9) —(CH₂)ₛC(O)NR^{e}R^{j},
10) —(CH₂)ₛNR^{e}C(O)R^{j},
11) —(CH₂)ₛNR^{e}C(O)OR^{j},
12) —(CH₂)ₛNR^{e}C(O)N(R^{e})₂,
13) —(CH₂)ₛNR^{e}C(O)NR^{e}R^{j},
14) —(CH₂)ₛNR^{e}S(O)ₘR^{j},
15) —(CH₂)ₛNR^{e}S(O)mN(R^{e})₂,
16) —(CH₂)ₛNR^{e}S(O)mNR^{e}R^{j}, and
17) —(CH₂)ₛNR^{e}R^{j},
   wherein each CH₂, alkyl, alkenyl, alkynyl, cycloalkyl, and cycloheteroalkyl is unsubstituted or substituted with one to five substituents selected from R^{g}, and
   wherein R⁵ and R⁴ and the carbon atom they are connected to can form a —C₃₋₅cycloalkyl ring, or wherein R⁵ and R⁶ and the carbon atoms they are connected to can form a —C₃₋₅cycloalkyl ring;
   R⁶ is selected from the group consisting of:
1) hydrogen, and
2) —C₁₋₆alkyl,
   wherein each alkyl is unsubstituted or substituted with one to five halogen substituents; R⁷ is selected from the group consisting of:
1) hydrogen,
2) —C₁₋₆alkyl,
3) —C₃₋₆cycloalkyl, and
4) —C₂₋₆cycloheteroalkyl,
   wherein each alkyl, cycloalkyl and cycloheteroalkyl is unsubstituted or substituted with one to five halogen substituents;
   R⁸ is selected from the group consisting of:
1) hydrogen,
2) —C₁₋₆alkyl,
3) —C₂₋₆alkenyl, and
4) —C₂₋₆alkynyl,
   wherein each alkyl, alkenyl and alkynyl is unsubstituted or substituted with one to five halogen substituents;
   each R^{a} is independently selected from the group consisting of:
1) —CF₃,
2) —OCF₃,
3) —CHF₂,
4) —OCHF₂,
5) —CH₂CF₃,
6) —OCH₂CF₃,
7) —CF₂CH₃
8) CN,
9) oxo,
10) halogen,
11) —S(O)₂C₁₋₆alkyl,
12) —C₁₋₆alkyl,
13) —C₂₋₆alkenyl,
14) —C₂₋₆alkynyl,
15) —C₃₋₆cycloalkyl,
16) —C₂₋₆cycloheteroalkyl,
17) aryl,
18) heteroaryl,
19) —C₁₋₆alkyl-aryl,
20) —C₁₋₆alkyl-heteroaryl,
21) —C₁₋₆alkyl-C₃₋₆cycloalkyl,
22) —C₁₋₆alkyl-C₂₋₆cycloheteroalkyl,
23) —C₂₋₆alkenyl-C₃₋₆cycloalkyl,
24) —C₂₋₆alkenyl-C₂₋₆cycloheteroalkyl,
25) —C₂₋₆alkenyl-aryl,
26) —C₂₋₆alkenyl-heteroaryl,
27) —C₂₋₆alkynyl-C₃₋₆cycloalkyl,
28) —C₂₋₆alkynyl-C₂₋₆cycloheteroalkyl,
29) —C₂₋₆alkynyl-aryl,
30) —C₂₋₆alkynyl-heteroaryl,
31) —OH,
32) —(CH₂)ₚ—OC₁₋₆alkyl,
33) —(CH₂)ₚ—OC₂₋₆alkenyl,
34) —(CH₂)ₚ—OC₂₋₆alkynyl,
35) —(CH₂)ₚ—OC₃₋₆cycloalkyl,
36) —(CH₂)ₚ—OC₂₋₆heterocycloalkyl,
37) —(CH₂)ₚ—O-aryl,
38) —(CH₂)ₚ—O-heteroaryl,
39) —OC₁₋₆alkyl-C₃₋₆cycloalkyl,
40) —OC₁₋₆alkyl-C₂₋₆ heterocycloalkyl,
41) —OC₁₋₆alkyl-aryl,
42) —OC₁₋₆alkyl-heteroaryl,
43) —S(O)ₘR^{j},
44) —C₁₋₆alkyl-S(O)ₘR^{j},
45) —N(R^{k})₂, and
46) —NR^{k}R^{L},
   wherein each R^{a} is unsubstituted or substituted with one to six substituents selected from halogen, CF₃, OH, C₁₋₆alkyl, and —OC₁₋₆alkyl;
   each R^{b} is independently selected from the group consisting of:
1) —CF₃,
2) —OCF₃,
3) —CHF₂,
4) —OCHF₂,
5) —CH₂CF₃,
6) —OCH₂CF₃,
7) —CF₂CH₃
8) CN,
9) oxo,
10) halogen,
11) —S(O)₂C₁₋₆alkyl,
12) —C₁₋₆alkyl,
13) —C₂₋₆alkenyl,
14) —C₂₋₆alkynyl,
15) —O—C₁₋₆alkyl,
16) —C₃₋₆cycloalkyl,
17) —O—C₃₋₆cycloalkyl,
18) —C₂₋₆cycloheteroalkyl,
19) aryl,
20) heteroaryl,
21) —C₁₋₆alkyl-aryl,
22) —C₁₋₆alkyl-heteroaryl,
23) —C₁₋₆alkyl-C₃₋₆cycloalkyl,
24) —C₁₋₆alkyl-C₂₋₆cycloheteroalkyl,
25) —C₂₋₆alkenyl-C₃₋₆cycloalkyl,
26) —C₂₋₆alkenyl-C₂₋₆cycloheteroalkyl,
27) —C₂₋₆alkenyl-aryl,
28) —C₂₋₆alkenyl-heteroaryl,
29) —C₂₋₆alkynyl-C₃₋₆cycloalkyl,
30) —C₂₋₆alkynyl-C₂₋₆cycloheteroalkyl,
31) —C₂₋₆alkynyl-aryl,
32) —C₂₋₆alkynyl-heteroaryl,
33) —OH,
34) —(CH₂)_{q}—OC₁₋₆alkyl,
35) —(CH₂)_{q}—OC₂₋₆alkenyl,
36) —(CH₂)_{q}—OC₂₋₆alkynyl,
37) —(CH₂)_{q}—OC₃₋₆cycloalkyl,
38) —(CH₂)_{q}—OC₂₋₆heterocycloalkyl,
39) —(CH₂)_{q}—O-aryl,
40) —(CH₂)_{q}—O-heteroaryl,
41) —OC₁₋₆alkyl-C₃₋₆cycloalkyl,
42) —OC₁-6alkyl-C₂₋₆heterocycloalkyl,
43) —OC₁₋₆alkyl-aryl,
44) —OC₁₋₆alkyl-heteroaryl,
45) —S(O)ₘR^{j},
46) —C₁₋₆alkyl-S(O)ₘR^{j},
47) —C(O)R^{L}, and
48) —NR^{k}R^{L},
   wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl;
   R^{c} is selected from:
1) —C₁₋₆alkyl,
2) OH,
3) halogen, and
4) —OC₁₋₆alkyl,
   wherein alkyl can be unsubstituted or substituted with one to three halogens;
   R^{d} is selected from:
1) —C₁₋₆alkyl,
2) OH,
3) halogen, and
4) —OC₁₋₆alkyl,
   wherein alkyl can be unsubstituted or substituted with one to three halogens;
   R^{e} is selected from:
1) hydrogen, and
2) C₁₋₆alkyl;
   R^{f} is selected from:
1) —C₁₋₆alkyl,
2) OH,
3) halogen, and
4) —OC₁₋₆alkyl,
   wherein alkyl can be unsubstituted or substituted with one to three halogens;
   R^{g} is selected from:
1) —C₁₋₆alkyl,
2) OH,
3) halogen, and
4) —OC₁₋₆alkyl,
   wherein alkyl can be unsubstituted or substituted with one to three halogens;
   R^{h} is selected from:
1) hydrogen, and
2) C₁₋₆alkyl;
   Rⁱ is selected from:
1) hydrogen,
2) C₁₋₆alkyl,
3) C₃₋₆cycloalkyl,
4) aryl, and
5) heteroaryl;
   R^{j} is selected from:
1) hydrogen,
2) C₁₋₆alkyl,
3) C₃₋₆alkenyl,
4) C₃₋₆alkynyl,
5) C₃₋₆cycloalkyl,
6) C₂₋₅cycloheteroalkyl,
7) aryl, and
8) heteroaryl;
   R^{k} is selected from:
1) hydrogen, and
2) C₁₋₆alkyl;
   R^{L} is selected from:
1) hydrogen,
2) C₁₋₆alkyl,
3) C₃₋₆cycloalkyl,
4) aryl, and
5) heteroaryl;
   m is independently selected from 0 to 2;
   n is independently selected from 2 to 6;
   p is independently selected from 0 to 3;
   q is independently selected from 0 to 3;
   r is independently selected from 0 to 2; and
   s is independently selected from 0 to 6;
   or a pharmaceutically acceptable salt thereof.

Another embodiment of the present invention relates to compounds of structural formula I wherein:

A is aryl, wherein aryl is unsubstituted or substituted with one to five substituents selected from R^{a};
B is independently selected from the group consisting of:
1) aryl,
2) heteroaryl,
3) —C₁₋₆alkyl-aryl,
4) —C₁₋₆alkyl-O-aryl, and
5) —C₃₋₁₂cycloalkyl,
   wherein alkyl, cycloalkyl, aryl and heteroaryl are unsubstituted or substituted with one to five substituents selected from R^{b};
   R¹ is selected from the group consisting of:
1) hydrogen, and
2) —C₁₋₆alkyl,
   wherein each alkyl is unsubstituted or substituted with one to five substituents selected from R^{c};
   R² is selected from the group consisting of:
1) hydrogen, and
2) —C₁₋₆alkyl,
   wherein each alkyl is unsubstituted or substituted with one to five substituents selected from R^{d};
   R³ is selected from the group consisting of:
1) hydrogen, and
2) —C₁₋₆alkyl,
   wherein each alkyl is unsubstituted or substituted with one to five substituents selected from R^{d};
   R⁴ is selected from the group consisting of:
1) hydrogen, and
2) —C₁₋₆alkyl,
   wherein each alkyl is unsubstituted or substituted with one to five substituents selected from R^{g}; R⁵ is selected from the group consisting of:
1) hydrogen, and
2) —C₁₋₆alkyl,
   wherein each alkyl is unsubstituted or substituted with one to five substituents selected from R^{g};
   R⁶ is hydrogen;
   R⁷ is selected from the group consisting of:
1) hydrogen, and
2) —C₁₋₆alkyl,
   wherein each alkyl is unsubstituted or substituted with one to five halogen substituents;
   R⁸ is selected from the group consisting of:
1) hydrogen, and
2) —C₁₋₆alkyl,
   wherein each alkyl is unsubstituted or substituted with one to five halogen substituents; each R^{a} is independently selected from the group consisting of:
1) —CF₃,
2) —OCF₃, and
3) halogen;
   each R^{b} is independently selected from the group consisting of:
1) —CF₃,
2) —OCF₃,
3) —OCHF₂,
4) CN,
5) halogen,
6) —C₁₋₆alkyl,
7) —O—C₁₋₆alkyl, and
8) —C₃₋₆cycloalkyl,
   wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl;
   or a pharmaceutically acceptable salt thereof.

Another embodiment of the present invention relates to compounds of structural formula I wherein:

A is phenyl, wherein phenyl is unsubstituted or substituted with one to five substituents selected from R^{a};
B is independently selected from the group consisting of:
1) phenyl,
2) pyridine,
3) thiazole,
4) —(CH₂)₂-phenyl,
5) —CH₂—O-phenyl, and
6) cyclobutane,
   wherein B is unsubstituted or substituted with one to five substituents selected from R^{b};
   R¹, R², R³, R⁴, R⁵, R⁶, R⁷ and R⁸ are hydrogen;
   each R^{a} is independently selected from the group consisting of:
1) —CF₃, and
2) halogen;
   each R^{b} is independently selected from the group consisting of:
1) —CF₃, and
2) halogen;
   or a pharmaceutically acceptable salt thereof.

Illustrative, but non-limiting, examples of the compounds of the present invention that are useful as inhibitors of Naᵥ1.8 channel activity are the following compounds:
1) (S)—N—OR)-(3-chloro-4-fluorophenyl)(4-chlorophenyl)methyl)-5-oxopyrrolidine-3-carboxamide;
2) (S)—N—((S)-(3-chloro-4-fluorophenyl)(4-chlorophenyl)methyl)-5-oxopyrrolidine-3-carboxamide;
3) (3S)—N-((3-chlorophenyl)(3-cyanophenyl)methyl)-5-oxopyrrolidine-3-carboxamide;
4) (3S)—N-((4-chlorophenyl)(4-cyclopropylphenyl)methyl)-5-oxopyrrolidine-3-carboxamide;
5) (3S)—N-((4-chlorophenyl)(4-isopropylphenyl)methyl)-5-oxopyrrolidine-3-carboxamide;
6) (3S)—N-((4-chloro-2-methoxyphenyl)(4-chlorophenyl)methyl)-5-oxopyrrolidine-3-carboxamide;
7) (3S)—N-((3-chlorophenyl)(3-(difluoromethoxy)phenyl)methyl)-5-oxopyrrolidine-3-carboxamide;
8) (3S)—N-((4-chlorophenyl)(3-fluoro-5-(trifluoromethyl)phenyl)methyl)-5-oxopyrrolidine-3-carboxamide;
9) (3S)-5-oxo-N-((3-(trifluoromethyl)phenyl)(4-(trifluoromethyl)phenyl)methyl)pyrrolidine-3-carboxamide;
10) (3S)-5-oxo-N-((3-(trifluoromethyl)phenyl)(4-(trifluoromethyl)phenyl)methyl)pyrrolidine-3-carboxamide;
11) (S)—N-(bis(4-(trifluoromethyl)phenyl)methyl)-5-oxopyrrolidine-3-carboxamide;
12) (3S)—N-((4-chlorophenyl)(3-(trifluoromethoxy)phenyl)methyl)-5-oxopyrrolidine-3-carboxamide;
13) (S—N—((R)-(4-chlorophenyl)(4-fluoro-3-(trifluoromethyl)phenyl)methyl)-5-oxopyrrolidine-3-carboxamide;
14) (S)—N—((S)-(4-chlorophenyl)(4-fluoro-3-(trifluoromethyl)phenyl)methyl)-5-oxopyrrolidine-3-carboxamide;
15) (S—N—((R)-(4-chlorophenyl)(4-(trifluoromethoxy)phenyl)methyl)-5-oxopyrrolidine-3-carboxamide;
16) (S)—N—((S)-(4-chlorophenyl)(4-(trifluoromethoxy)phenyl)methyl)-5-oxopyrrolidine-3-carboxamide;
17) (S)—N—OR)-(3-chloro-4-fluorophenyl)(4-cyanophenyl)methyl)-5-oxopyrrolidine-3-carboxamide;
18) (S)—N—((S)-(3-chloro-4-fluorophenyl)(4-cyanophenyl)methyl)-5-oxopyrrolidine-3-carboxamide;
19) (S)—N—OR)-(4-chloro-3-(trifluoromethyl)phenyl)(4-chlorophenyl)methyl)-5-oxo-pyrrolidine-3-carboxamide;
20) (S)—N—((S)-(4-chloro-3-(trifluoromethyl)phenyl)(4-chlorophenyl)methyl)-5-oxo-pyrrolidine-3-carboxamide;
21) (S)—N—OR)-(3-chloro-2,4-difluorophenyl)(5-chloro-6-(trifluoromethyl)pyridin-3-yl)methyl)-5-oxopyrrolidine-3-carboxamide;
22) (S)—N—((S)-(3-chloro-2,4-difluorophenyl)((trans)-3-(trifluoromethyl)cyclobutyl)-methyl)-5-oxopyrrolidine-3-carboxamide;
23) (S)—N—OR)-(3-chloro-4-fluorophenyl)(4-(trifluoromethyl)phenyl)methyl)-5-oxo-pyrrolidine-3-carboxamide;
24) (S)—N—((S)-(3-chloro-4-fluorophenyl)(4-(trifluoromethyl)phenyl)methyl)-5-oxo-pyrrolidine-3-carboxamide;
25) (S)—N-(bis(3-chloro-4-fluorophenyl)methyl)-5-oxopyrrolidine-3-carboxamide;
26) (S—N—((R)-(3-chlorophenyl)(4-(trifluoromethoxy)phenyl)methyl)-5-oxopyrrolidine-3-carboxamide;
27) (S)—N—((S)-(3-chlorophenyl)(4-(trifluoromethoxy)phenyl)methyl)-5-oxopyrrolidine-3-carboxamide;
28) (S)—N—OR)-(3-chloro-4-fluorophenyl)(5-chloro-6-(trifluoromethyl)-pyridin-2-yl)-methyl)-5-oxopyrrolidine-3-carboxamide;
29) (S)—N—((S)-(3-chloro-4-fluorophenyl)(5-chloro-6-(trifluoromethyl)-pyridin-2-yl)-methyl)-5-oxopyrrolidine-3-carboxamide;
30) (S—N—((R)-(3-chloro-2,4-difluorophenyl)(3,3-dimethylcyclobutyl)methyl)-5-oxo-pyrrolidine-3-carboxamide;
31) (S)—N—((S)-(3-chloro-2,4-difluorophenyl)(3,3-dimethylcyclobutyl)methyl)-5-oxo-pyrrolidine-3-carboxamide;
32) (3S)—N-((3-chloro-4-fluorophenyl)(3-cyano-4-fluorophenyl)methyl)-5-oxopyrrolidine-3-carboxamide;
33) (3S)—N-((4-fluoro-3-(trifluoromethyl)phenyl)(2-(trifluoromethyl)thiazol-4-yl)methyl)-5-oxopyrrolidine-3-carboxamide;
34) (3S)—N-((3-chloro-2,4-difluorophenyl)(4-fluoro-3-(trifluoromethyl)phenyl)methyl)-5-oxopyrrolidine-3-carboxamide;
35) (3S)—N-(1-(4-fluoro-3-(trifluoromethyl)phenyl)-2-phenoxyethyl)-5-oxopyrrolidine-3-carboxamide;
36) (3S)—N-(1-(3-chlorophenyl)-3-phenylpropyl)-5-oxopyrrolidine-3-carboxamide;
37) (3R,4R)—N-(bis(4-chlorophenyl)methyl)-4-methyl-5-oxopyrrolidine-3-carboxamide;
38) (3R,4S)—N-(bis(4-chlorophenyl)methyl)-4-methyl-5-oxopyrrolidine-3-carboxamide;
39) (3S,4S)—N-(bis(4-chlorophenyl)methyl)-4-methyl-5-oxopyrrolidine-3-carboxamide; and
40) (3S,4R)—N-(bis(4-chlorophenyl)methyl)-4-methyl-5-oxopyrrolidine-3-carboxamide;
or a pharmaceutically acceptable salt thereof.

Additional illustrative, but non-limiting, examples of the compounds of the present invention that are useful as inhibitors of Naᵥ1.8 channel activity are the following compounds:
1) (S)—N—OR)-(3-chloro-4-fluorophenyl)(4-chlorophenyl)methyl)-5-oxopyrrolidine-3-carboxamide;
2) (S)—N—((S)-(3-chloro-4-fluorophenyl)(4-chlorophenyl)methyl)-5-oxopyrrolidine-3-carboxamide;
3) (S)—N—OR)-(4-chloro-3-(trifluoromethyl)phenyl)(4-chlorophenyl)methyl)-5-oxo-pyrrolidine-3-carboxamide; and
4) (S)—N—((S)-(4-chloro-3-(trifluoromethyl)phenyl)(4-chlorophenyl)methyl)-5-oxo-pyrrolidine-3-carboxamide;
   or a pharmaceutically acceptable salt thereof.

Although the specific stereochemistries described above are preferred, other stereoisomers, including diastereoisomers, enantiomers, epimers, and mixtures of these may also have utility in treating Naᵥ1.8 mediated diseases.

Synthetic methods for making the compounds are disclosed in the Examples shown below. Where synthetic details are not provided in the examples, the compounds are readily made by a person of ordinary skill in the art of medicinal chemistry or synthetic organic chemistry by applying the synthetic information provided herein. Where a stereochemical center is not defined, the structure represents a mixture of stereoisomers at that center. For such compounds, the individual stereoisomers, including enantiomers, diastereoisomers, and mixtures of these are also compounds of the invention.

### Definitions

“Ac” is acetyl, which is CH₃C(═O)—.

“Alkyl” means saturated carbon chains which may be linear or branched or combinations thereof, unless the carbon chain is defined otherwise. Other groups having the prefix “alk”, such as alkoxy and alkanoyl, also may be linear or branched, or combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like.

“Alkenyl” means carbon chains which contain at least one carbon-carbon double bond, and which may be linear or branched, or combinations thereof, unless otherwise defined. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched, or combinations thereof, unless otherwise defined. Examples of alkynyl include ethynyl, propargyl, 3-methyl-1-pentynyl, 2-heptynyl and the like.

“Cycloalkyl” means a saturated monocyclic, bicyclic, spirocyclic or bridged carbocyclic ring, having a specified number of carbon atoms. Examples of cycloalkyl include cyclopropyl, cyclobutyl, cyclopentyl, cyclohexyl, cycloheptyl, and the like. In one embodiment of the present invention, cycloalkyl is selected from: cyclopropyl, cyclobutyl and cyclohexyl. In another embodiment, cycloalkyl is cyclopropyl, cyclobutyl or cyclopentyl. In another embodiment, cycloalkyl is cyclopropyl or cyclobutyl. In another embodiment, cycloalkyl is cyclopropyl. In another embodiment, cycloalkyl is cyclobutyl. In another embodiment, cycloalkyl is cyclopentyl. In another embodiment, cycloalkyl is cyclohexyl. In another embodiment, cycloalkyl is cycloheptyl.

“Cycloalkenyl” means a monocyclic, bicyclic, spirocyclic or bridged carbocyclic ring, having a specified number of carbon atoms with at least one double bond. Examples of cycloalkenyl include cyclopropenyl, cyclobutenyl, cyclopentenyl, cycloheptenyl, and the like. In one embodiment, cycloalkenyl is cyclobutenyl.

“Cycloheteroalkyl” means a saturated or partly unsaturated non-aromatic monocyclic, bicyclic, spirocyclic or bridged ring or ring system having a specified number of carbon atoms and containing at least one ring heteroatom selected from N, NH, S (including SO and SO₂) and O. The cycloheteroalkyl ring may be substituted on the ring carbons and/or the ring nitrogen or sulfur. Examples of cycloheteroalkyl include tetrahydrofuranyl, pyrrolidinyl, tetrahydrothiophenyl, azetidinyl, piperazinyl, piperidinyl, morpholinyl, oxetanyl and tetrahydropyranyl. In one embodiment of the present invention, cycloheteroalkyl is selected from: pyrrolidinyl, azetidinyl, piperidine, piperazine, azepane, azocane, morpholine, thiomorpholine, thiomorpholine dione, oxazepanyl, 1,4-thiazepanyl, isoindolinyl, dihydroisoquinolinyyl, tetra-hydroisoquinolinyl, octahydro-isoindolyl, azabicyclo[2.2.1]heptanyl, oxa-azabicyclo[2.2.1]-heptanyl, azabicyclo[3.1.1]heptane, azabicyclo[4.1.0]heptanyl, azabicyclo[3.2.1]octane, diazabicyclo[3.2.1]octane, oxa-azabicyclo-[3.2.1]octane, azabicyclo[3.2.0]heptane, oxa-azabicyclo[3.2.0]heptane, azaspiro[2.5]octane, azaspiro[2.6]nonane, azaspiro[3.5]nonane, oxa-azaspiro[3.5]nonane, oxa-azaspiro[4.5]decane, dihydrothieno[3,2-c]pyridine, dihydro-thiazolo[4,5-c]pyridine, dihydrooxazolo[4,5-c]pyridine, dihydroimidazo[1,2-a]pyrazine, hexahydrofuro[3,2-b]pyrrole, hexahydrocyclopenta[c]pyrrole, octahydrocyclpenta[c]pyrrole, and azatricyclo[4.3.1.13,8]undecane. In another embodiment, cycloheteroalkyl is selected from: pyrrolidine, azetidine, piperidine, piperazine, azepane, morpholine, thiomorpholine, oxazepane, isoindoline, dihydroisoquinoline, azabicyclo[2.2.1]heptane, azabicyclo[3.1.1]-heptane, azabicyclo[4.1.0]heptane, azabicyclo[3.2.1]octane, azabicyclo[3.2.0]heptane, azaspiro[2.5]octane, dihydrothieno[3,2-c]pyridine, dihydroimidazo[1,2-a]pyrazine, and hexahydrofuro[3,2-b]pyrrole. In another embodiment, cycloheteroalkyl is selected from: azepane, morpholine and piperidine. In another embodiment, cycloheteroalkyl is azepane. In another embodiment, cycloheteroalkyl is morpholine. In another embodiment, cycloheteroalkyl is piperidine.

“Cycloheteroalkenyl” means a monocyclic, bicyclic, spirocyclic or bridged ring or ring system having a specified number of carbon atoms and containing at least one double bond and at least one heteroatom. Examples of cycloheteroalkenyl include dihydropyran and dihydrofuran, and the like.

“Aryl” means a monocyclic, bicyclic or tricyclic carbocyclic aromatic ring or ring system containing 6-14 carbon atoms, wherein at least one of the rings is aromatic. Examples of aryl include phenyl and naphthyl. In one embodiment of the present invention, aryl is phenyl.

“Heteroaryl” means a monocyclic, bicyclic or tricyclic ring or ring system containing 5-14 ring atoms and containing at least one ring heteroatom selected from N, NH, S (including SO and SO₂) and O, wherein at least one of the heteroatom containing rings is aromatic. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzisoxazolyl, benzoxazolyl, benzothiazolyl, benzimidazolyl, benzofuranyl, benzothiophenyl, quinolyl, indolyl, isoquinolyl, quinazolinyl, dibenzofuranyl, and the like. In one embodiment of the present invention, heteroaryl is a 5 or 6 membered heteroaryl ring. In another embodiment, heteroaryl is selected from: pyrazolyl, pyridyl, isoxazolyl and thiazolyl. In another embodiment of the present invention, heteroaryl is selected from: pyridinyl, pyrimidinyl, pyrazinyl, pyridazinyl, indazolyl, imidazo[1,2-a]pyridinyl, 1,3-dihydro-2H-imidazo[4,5-b]pyridin-2-one, 1H-[1,2,3]triazolo[4,5-b]pyridinyl, 1H-pyrazolo[4,3-b]pyridinyl, pyrrolo[3,2-c]pyridinyl, pyrrolo[2,3-b]pyridinyl, benzimidazolyl, imidazolyl, pyrazolyl, thiophenyl, furanyl, 1,2,4-oxadiazolyl, 1,3,4-oxadiazolyl, oxazolyl, isoxazolyl, isothiazolyl, thiazolyl, 1,2,4-thiadiazolyl, 1,3,4-thiadiazolyl; 4H-pyrido[2,3-e][1,2,4]thiadiazinyl 1,1-dioxide, 2H-pyrido[2,3-e][1,2]thiazinyl 1,1-dioxide, 2,3-dihydroisothiazolo[4,5-b]pyridinyl 1,1-dioxide, and 3,4-dihydro-2H-pyrido[2,3-e][1,2]thiazinyl 1,1-dioxide. In another embodiment of the present invention, heteroaryl is selected from: pyridinyl, pyrimidinyl, and pyridazinyl. In another embodiment of the present invention, heteroaryl is pyridinyl. In another embodiment heteroaryl is pyridine or thiazole.

“Halogen” includes fluorine, chlorine, bromine and iodine. In one embodiment, halogen is fluorine, chorine or bromine. In another embodiment, halogen is fluorine or chlorine. In another embodiment, halogen is fluorine or bromine. In another embodiment, halogen is fluorine. In another embodiment, halogen is chlorine. In another embodiment, halogen is bromine.

“Me” represents methyl.

“Oxo” represents ═O.

“Saturated” means containing only single bonds.

“Unsaturated” means containing at least one double or triple bond. In one embodiment, unsaturated means containing at least one double bond. In another embodiment, unsaturated means containing at least one triple bond.

When any variable (e.g., R¹, R^{a}, etc.) occurs more than one time in any constituent or in formula I, its definition on each occurrence is independent of its definition at every other occurrence. Also, combinations of substituents and/or variables are permissible only if such combinations result in stable compounds. A squiggly line across a bond in a substituent variable represents the point of attachment.

Under standard nomenclature used throughout this disclosure, the terminal portion of the designated side chain is described first, followed by the adjacent functionality toward the point of attachment. For example, a C₁₋₅alkylcarbonylamino C₁₋₆alkyl substituent is equivalent to:

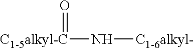

In choosing compounds of the present invention, one of ordinary skill in the art will recognize that the various substituents, i.e. R¹, R², etc., are to be chosen in conformity with well-known principles of chemical structure connectivity and stability.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The phrase “pharmaceutically acceptable” is employed herein to refer to those compounds, materials, compositions, salts and/or dosage forms which are, using sound medical judgment, and following all applicable government regulations, safe and suitable for administration to a human being or an animal.

Compounds of Formula I may contain one or more asymmetric centers and can thus occur as racemates and racemic mixtures, single enantiomers, diastereomeric mixtures and individual diastereomers. The present invention is meant to encompass all such isomeric forms of the compounds of Formula I.

The independent syntheses of optical isomers and diastereoisomers or their chromatographic separations may be achieved as known in the art by appropriate modification of the methodology disclosed herein. Their absolute stereochemistry may be determined by the X-ray crystallography of crystalline products or crystalline intermediates which are derivatized, if necessary, with a reagent containing an asymmetric center of known absolute configuration or sufficient heavy atoms to make an absolute assignment.

If desired, racemic mixtures of the compounds may be separated so that the individual enantiomers are isolated. The separation can be carried out by methods well-known in the art, such as the coupling of a racemic mixture of compounds to an enantiomerically pure compound to form a diastereoisomeric mixture, followed by separation of the individual diastereoisomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

Some of the compounds described herein contain olefinic double bonds, and unless specified otherwise, are meant to include both E and Z geometric isomers.

Tautomers are defined as compounds that undergo rapid proton shifts from one atom of the compound to another atom of the compound. Some of the compounds described herein may exist as tautomers with different points of attachment of hydrogen. Such an example may be a ketone and its enol form known as keto-enol tautomers. The individual tautomers as well as mixture thereof are encompassed with compounds of Formula I.

In the compounds of general formula I, the atoms may exhibit their natural isotopic abundances, or one or more of the atoms may be artificially enriched in a particular isotope having the same atomic number, but an atomic mass or mass number different from the atomic mass or mass number predominately found in nature. The present invention is meant to include all suitable isotopic variations of the compounds of structural formula I. For example, different isotopic forms of hydrogen (H) include protium (CH), deuterium (²H), and tritium (³H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Tritium is radioactive and may therefore provide for a radiolabeled compound, useful as a tracer in metabolic or kinetic studies. Isotopically-enriched compounds within structural formula I, can be prepared without undue experimentation by conventional techniques well known to those skilled in the art or by processes analogous to those described in the Schemes and Examples herein using appropriate isotopically-enriched reagents and/or intermediates.

Furthermore, some of the crystalline forms for compounds of the present invention may exist as polymorphs and as such are intended to be included in the present invention. In addition, some of the compounds of the instant invention may form solvates with water or common organic solvents. Such solvates are encompassed within the scope of this invention.

It is generally preferable to administer compounds of the present invention as enantiomerically pure formulations. Racemic mixtures can be separated into their individual enantiomers by any of a number of conventional methods. These include chiral chromatography, derivatization with a chiral auxiliary followed by separation by chromatography or crystallization, and fractional crystallization of diastereomeric salts.

### Salts

It will be understood that, as used herein, references to the compounds of the present invention are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate/diphosphate, polygalacturonate, salicylate, stearate, sulfate, subacetate, succinate, tannate, tartrate, teoclate, tosylate, triethiodide, trifluoroacetate and valerate. Furthermore, where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as O-acetyl, O-pivaloyl, O-benzoyl, and O-aminoacyl, can be employed. Included are those esters and acyl groups known in the art for modifying the solubility or hydrolysis characteristics for use as sustained-release or prodrug formulations.

The term “prodrug” means compounds that are rapidly transformed, for example, by hydrolysis in blood, in vivo to the parent compound, e.g., conversion of a prodrug of Formula I to a compound of Formula I, or to a salt thereof; a thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference. This invention includes prodrugs of the novel compounds of this invention.

Solvates, and in particular, the hydrates of the compounds of the present invention are included in the present invention as well.

### Utilities

The compound of the present invention are selective inhibitors of Naᵥ1.8 sodium ion channel activity or have selective activity as Naᵥ1.8 sodium ion channel blockers. In one embodiment, the compounds of the present invention exhibit at least 10-fold selectivity for Naᵥ1.8 sodium channels over Naᵥ1.5 sodium channels, and in some embodiments exhibit at least 100-fold selectivity for Naᵥ1.8 sodium channels over Naᵥ1.5 sodium channels based on functional potency (IC₅₀ values) for each channel in Qube® assay system.

The compounds of the present invention are potent inhibitors of Naᵥ1.8 channel activity. The compounds, and pharmaceutically acceptable salts thereof, may be efficacious in the treatment of diseases, disorders and conditions that are mediated by the inhibition of Naᵥ1.8 sodium ion channel activity and/or Naᵥ1.8 receptors.

Diseases, disorders or conditions mediated by Naᵥ1.8 sodium ion channel activity and/or Naᵥ1.8 receptors, include but are not limited to nociception, osteoarthritis, peripheral neuropathy, inherited erythromelalgia, multiple sclerosis, asthma, pruritus, acute itch, chronic itch, migraine, neurodegeneration following ischemia, epilepsy, inflammatory pain, spontaneous pain, acute pain, peri-operative pain, post-operative pain, neuropathic pain, postherpetic neuralgia, trigeminal neuralgia, diabetic neuropathy, chronic lower back pain, phantom limb pain, pain resulting from cancer and chemotherapy, chronic pelvic pain, pain syndromes, and complex regional pain syndromes.

One or more of these conditions or diseases may be treated, managed, prevented, reduced, alleviated, ameliorated or controlled by the administration of a therapeutically effective amount of a compound of the present invention, or a pharmaceutically acceptable salt thereof, to a patient in need of treatment. Also, the compounds of the present invention may be used for the manufacture of a medicament which may be useful for treating, preventing, managing, alleviating, ameliorating or controlling one or more of these conditions, diseases or disorders: nociception, osteoarthritis, peripheral neuropathy, inherited erythromelalgia, multiple sclerosis, asthma, pruritus, acute itch, chronic itch, migraine, neurodegeneration following ischemia, epilepsy, inflammatory pain, spontaneous pain, acute pain, pen-operative pain, post-operative pain, neuropathic pain, postherpetic neuralgia, trigeminal neuralgia, diabetic neuropathy, chronic lower back pain, phantom limb pain, pain resulting from cancer and chemotherapy, chronic pelvic pain, pain syndromes, and complex regional pain syndromes.

Preferred uses of the compounds may be for the treatment of one or more of the following diseases by administering a therapeutically effective amount to a patient in need of treatment. The compounds may be used for manufacturing a medicament for the treatment of one or more of these diseases:
1) pain conditions,
2) pruritic conditions, and
3) cough conditions.

In one embodiment of the present invention, the pain condition is an acute pain or chronic pain disorder. In another embodiment of the present invention, the the pain condition is an acute pain disorder.

The compounds of the present invention may be effective in treating nociception. Nociception or pain is essential for survival and often serves a protective function. However, the pain associated with surgical procedures and current therapies to relieve that pain, can delay recovery after surgery and increase the length of hospital stays. As many as 80% of surgical patients experience post-operative pain due to tissue damage, and damage to peripheral nerves and subsequent inflammation. Approximately 10-50% of surgical patients will develop chronic pain after surgery often because the nerve damage results in lasting neuropathic pain once the wound has healed.

The compounds of the present invention may be effective in treating osteoarthritis. Osteoarthritis is type of arthritis caused by inflammation, breakdown, and eventual loss of cartilage in the joints. The standards of care for pain associated with osteoarthritis are non-steroidal anti-inflammatory drugs (NSAIDs), for example celecoxib and diclofenac (reviewed in Zeng et al., 2018). Patients that do not respond to NSAID therapies are typically treated with low dose opiates, such as hydrocodone. Patients that are refractory to the above therapies will usually opt for total joint replacement.

The compounds of the present invention may be effective in treating peripheral neuropathy. Peripheral neuropathy is nerve damage caused by chronically high blood sugar and diabetes. It leads to numbness, loss of sensation, and sometimes pain in distal limbs such as feet, legs, or hands. It is the most common complication of diabetes. The standards of care for the treatment of painful diabetic neuropathy are gabapentinoids, for example gabapentin and pregabalin. Some patients will respond well to tricyclic antidepressants such as amitriptyline, while other patients get significant relief using SRI/NRI drugs such as duloxetine (Schreiber et al., World J Diabetes. 2015 Apr. 15; 6(3):432-44). Many options are available, however side-effects are common (e.g. dizziness, nausea) which limit their full potential.

The compounds of the present invention may be effective in treating inherited erythromelalgia. Inherited erythromelalgia (IEM) is a chronic pain syndrome which has been linked to mutations in several voltage-gated sodium channels, including Nav1.8 (Kist et al., PLoS One. 2016 Sep. 6; 11(9):e0161789). Patients present with the classic “gloves and stocking” flare pattern on distal regions such as hands and feet, typically brought on with warm temperatures and exercise. Some patients find relief from the burning pain associated with flares by cold water immersion. Although medications that affect voltage-gated sodium channels (eg, lidocaine and mexiletine) show promise, there is no current standard of care to treat IEM.

The compounds of the present invention may be effective in treating neuropathic pain. Neuropathic pain is pain caused by damage or disease affecting the somatosensory nervous system. It has been demonstrated in human patients, as well as in animal models of neuropathic pain, that damage to primary afferent sensory neurons can lead to neuroma formation and spontaneous activity, as well as evoked activity in response to normally innocuous stimuli. (Colloca et al., Nat Rev Dis Primers. 2017 Feb. 16; 3:17002; Coward et al., Pain. 2000 March; 85(1-2):41-50; Yiangou et al., FEBS Lett. 2000 Feb. 11; 467(2-3):249-52; Carter et al., Phys Med Rehabil Clin N Am. 2001 May; 12(2):447-59). Some nerve injuries result in an increase in Nav1.8 expression, which is believed to be an underlying mechanism for pathological pain. (Black et al., Ann Neurol. 2008 December; 64(6):644-53; Bird et al., Br J Pharmacol. 2015 May; 172(10):2654-70). Injuries of the peripheral nervous system often result in neuropathic pain persisting long after an initial injury resolves. Examples of neuropathic pain include, but are not limited to, post herpetic neuralgia, trigeminal neuralgia, diabetic neuropathy, chronic lower back pain, lumbar radiculopathy, phantom limb pain, pain resulting from cancer and chemotherapy, chronic pelvic pain, complex regional pain syndrome and related neuralgias, and painful conditions that arise due to gain-of-function mutations in Nav1.8 (Huang et al., J Neurosci. 2013 Aug. 28; 33(35):14087-97; Kist et al., PLoS One. 2016 Sep. 6; 11(9):e0161789; Emery et al., J Neurosci. 2015 May 20; 35(20):7674-81; and Schreiber et al., World J Diabetes. 2015 Apr. 15; 6(3):432-44.

The ectopic activity of normally silent sensory neurons is thought to contribute to the generation and maintenance of neuropathic pain, which is generally assumed to be associated with an increase in sodium channel activity in the injured nerve. (Wood et al., Curr Opin Pharmacol. 2001 February; 1(1):17-21; Baker et al., TRENDS in Pharmacological Sciences, 2001, 22(1): 27-31). Standards of care for neuropathic pain vary considerably depending on the particular condition, but first line therapies are typically pregabalin, gabapentin, tricyclic antidepressants (e.g. amitriptyline), and SRI/NRI drugs (e.g. duloxetine). Patients refractory to these therapies are usually prescribed low dose opiates (e.g. hydrocodone).

The compounds of the present invention may be effective in treating multiple sclerosis. Recent evidence points to a potential role for Nav1.8 in multiple sclerosis. Nav1.8 expression in cerebellum has been identified in tissues taken from animal models of multiple sclerosis (EAE model) and in postmortem brains from patients suffering from multiple sclerosis (MS) (Shields et al., Ann Neurol. 2012 February; 71(2):186-94; Black et al., Proc Natl Acad Sci USA. 2000 Oct. 10; 97(21):11598-602). Also, two SCN10A polymorphisms showed significant association with MS (Roostaei et al., Neurology. 2016 Feb. 2; 86 (5):410-7). When Nav1.8 is overexpressed in cerebellum, mice develop ataxic-related motor deficits which are ameliorated with oral delivery of a selective small molecule Nav1.8 antagonist (Shields et al., PLoS One. 2015 Mar. 6; 10(3)). These studies suggest that a Nav1.8 antagonist may be a useful therapy to treat symptoms related to multiple sclerosis.

The compounds of the present invention may be effective in treating asthma. Asthma is caused by airway inflammation in which a person's airways become hyper-responsive, narrow and swollen, which makes it difficult to breathe. These symptoms are typically triggered through an allergic reaction (Nair P et al., J Allergy Clin Immunol Pract. 2017 May-June; 5(3):649-659). In a preclinical model of asthma, deletion of Nav1.8-containing neurons, or inhibition of nerve fibers via small molecules reduces airway inflammation and immune cell infiltration (Talbot et al., Neuron. 2015 Jul. 15; 87(2):341-54). Selective Nav1.8 antagonists may be a useful therapy to prevent airway hypersensitivity caused by immune cell infiltration.

The compounds of the present invention may be effective in treating pruritus. Pruritus, also commonly known as itch, affects approximately 4% of the global population is an unpleasant sensation that elicits the desire or reflex to scratch, and is regarded as closely related to pain (Luo et al., Cell Mol Life Sci. 2015 September; 72 (17): 3201-23). Theories on the origin of itch implicate the subtle, low-frequency activation of nociceptors (pain-sensing neurons); however, it has been described that some afferents preferentially respond to histamine, which induces itch (Schmelz et al., J Neurosci. 1997 Oct. 15; 17(20):8003-8). At the same time, it has been found that histamine-responding neurons also respond to capsaicin which produces pain (McMahon et al., Trends in Neuroscience 1992, 15:497-501). Members of the transient receptor potential (TRP) family, and nerve growth factor (NGF) are both known to play a role in itch and pain, and clinically, both maladies are treated with therapeutic agents such as gabapentin and antidepressants. Therefore, it continues to be accepted that the underlying mechanisms of pain and itch are highly interwoven and complex, and distinguishing pan-selective or itch-selective pathways remains ambiguous (Ikoma et al., Nat Rev Neurosci. 2006 July; 7(7):535-47). A role for Nav1.8 in pruritis was studied using a mouse transgenically expressing a constitutively active form of the serine/threonine kinase BRAF was expressed in Nav1.8-expressing neurons. This resulted in enhanced pruriceptor excitability, and heightened evoked and spontaneous scratching behavior (Zhao et al., 2013). In skin, pruritogens are released from keratinocytes, lymphocytes, mast cells, and eosinophils during inflammation. These molecules act directly on free nerve endings which express Nav1.8 to induce itch (Riol-Blanco et al., Nature. 2014 Jun. 5; 510 (7503):157-61). Chronic and acute itch can arise from many different insults, diseases and disorders, and may be classified as dermal or pruriceptive, neurogenic, neuropathic, or psychogenic: itch can arise from both systemic disorders, skin disorders, as well as physical or chemical insult to the dermis. Pathologically, conditions such as dry skin, eczema, psoriasis, varicella zoster, urticaria, scabies, renal failure, cirrhosis, lymphoma, iron deficiency, diabetes, menopause, polycythemia, uremia, and hyperthyroidism can cause itch, as can diseases of the nervous system such as tumors, multiple sclerosis, peripheral neuropathy, nerve compression, and delusions related to obsessive-compulsive disorders. Medicines such as opioids and chloroquine can also trigger itch (Ikoma et al., Nat Rev Neurosci. 2006 July; 7(7):535-47). Itching following burn is also an extremely serious clinical problem as it hampers the healing process, resulting in permanent scaring, and negatively impacting quality of life (Van Loey et al., Br J Dermatol. 2008 January; 158(1):95-100).

The invention also includes pharmaceutically acceptable salts of the compounds, and pharmaceutical compositions comprising the compounds and a pharmaceutically acceptable carrier.

The compounds, or pharmaceutically acceptable salts thereof, may be useful in treating pain conditions, pruritic conditions, and cough conditions.

A compound of the present invention, or a pharmaceutically acceptable salt thereof, may be used in the manufacture of a medicament for the treatment of pain conditions, pruritic conditions, and cough conditions in a human or other mammalian patient.

A method of treating a pain conditions comprises the administration of a therapeutically effective amount of a compound of the present invention, or a pharmaceutically acceptable salt thereof, or a pharmaceutical composition comprising the compound, to a patient in need of treatment. A method of treating a pruritic condition comprises the administration of a therapeutically effective amount of a compound of the present invention, or a pharmaceutically acceptable salt thereof, or a pharmaceutical composition comprising the compound, to a patient in need of treatment. A method of treating a cough condition comprises the administration of a therapeutically effective amount of a compound of the present invention, or a pharmaceutically acceptable salt thereof, or a pharmaceutical composition comprising the compound, to a patient in need of treatment. Other medical uses of the compounds of the present invention are described herein.

The term “pain condition” as used herein includes, but are not limited to, acute pain, peri-operative pain, pre-operative pain, post-operative pain, neuropathic pain, post herpetic neuralgia, trigeminal neuralgia, diabetic neuropathy, chronic lower back pain, phantom limb pain, chronic pelvic pain, vulvodynia, complex regional pain syndrome and related neuralgias, pain associated with cancer and chemotherapy, pain associated with HIV, and HIV treatment-induced neuropathy, nerve injury, root avulsions, painful traumatic mononeuropathy, painful polyneuropathy, erythromyelalgia, paroxysmal extreme pain disorder, small fiber neuropathy, burning mouth syndrome, central pain syndromes (potentially caused by virtually any lesion at any level of the nervous system), postsurgical pain syndromes (e.g., post mastectomy syndrome, post thoracotomy syndrome, stump pain)), bone and joint pain (osteoarthritis), repetitive motion pain, dental pain, myofascial pain (muscular injury, fibromyalgia), perioperative pain (general surgery, gynecological), chronic pain, dysmennorhea, pain associated with angina, inflammatory pain of varied origins (e.g. osteoarthritis, rheumatoid arthritis, rheumatic disease, teno-synovitis and gout), shoulder tendonitis or bursitis, gouty arthritis, and aolymyalgia rheumatica, primary hyperalgesia, secondary hyperalgesia, primary allodynia, secondary allodynia, or other pain caused by central sensitization, complex regional pain syndrome, chronic arthritic pain and related neuralgias acute pain, migraine, migraine headache, headache pain, cluster headache, non-vascular headache, traumatic nerve injury, nerve compression or entrapment, and neuroma pain,

The term “pruritic condition” or “pruritic disorder” as used herein includes, but is not limited to, conditions with an unpleasant sensation that provokes the desire to scratch, such as chronic itch.

The term “cough condition” or “cough disorder” as used herein includes, but is not limited to, chronic cough, neuropathic cough or cough due to neurological conditions.

Treatment of a disease, disorder or condition mediated by Naᵥ1.8 sodium ion channel activity or Naᵥ1.8 receptors refers to the administration of the compounds of the present invention to a subject with the disease, disorder or condition. One outcome of treatment may be reducing the disease, disorder or condition mediated by Naᵥ1.8 sodium ion channel activity or Naᵥ1.8 receptors. Another outcome of treatment may be alleviating the disease, disorder or condition mediated by Naᵥ1.8 sodium ion channel activity or Naᵥ1.8 receptors. Another outcome of treatment may be ameliorating the disease, disorder or condition mediated by Naᵥ1.8 sodium ion channel activity or Naᵥ1.8 receptors. Another outcome of treatment may be suppressing the disease, disorder or condition mediated by Naᵥ1.8 sodium ion channel activity or Naᵥ1.8 receptors. Another outcome of treatment may be managing the disease, disorder or condition mediated by Naᵥ1.8 sodium ion channel activity or Naᵥ1.8 receptors.

Another outcome of treatment may be preventing the disease, disorder or condition mediated by Naᵥ1.8 sodium ion channel activity or Naᵥ1.8 receptors.

Prevention of the disease, disorder or condition mediated by Naᵥ1.8 sodium ion channel activity or Naᵥ1.8 receptors refers to the administration of the compounds of the present invention to a subject at risk of the disease, disorder or condition. One outcome of prevention may be reducing the disease, disorder or condition mediated by Naᵥ1.8 sodium ion channel activity or Naᵥ1.8 receptors in a subject at risk of the disease, disorder or condition. Another outcome of prevention may be suppressing the disease, disorder or condition mediated by Naᵥ1.8 sodium ion channel activity or Naᵥ1.8 receptors in a subject at risk of the disease, disorder or condition. Another outcome of prevention may be ameliorating the disease, disorder or condition mediated by Naᵥ1.8 sodium ion channel activity or Naᵥ1.8 receptors in a subject at risk of the disease, disorder or condition. Another outcome of prevention may be alleviating the disease, disorder or condition mediated by Naᵥ1.8 sodium ion channel activity or Naᵥ1.8 receptors in a subject at risk of the disease, disorder or condition. Another outcome of prevention may be managing the disease, disorder or condition mediated by Naᵥ1.8 sodium ion channel activity or Naᵥ1.8 receptors in a subject at risk of the disease, disorder or condition.

One outcome of treatment may be reducing the amount of pain experienced by a subject relative to that subject's pain immediately before the administration of the compounds of the present invention. Another outcome of treatment may be alleviating the amount of pain experienced by a subject relative to that subject's pain immediately before the administration of the compounds of the present invention. Another outcome of treatment may be ameliorating the amount of pain experienced by a subject relative to that subject's pain immediately before the administration of the compounds of the present invention. Another outcome of treatment may be suppressing the amount of pain experienced by a subject relative to that subject's pain immediately before the administration of the compounds of the present invention. Another outcome of treatment may be managing the amount of pain experienced by a subject relative to that subject's pain immediately before the administration of the compounds of the present invention. Another outcome of treatment may be ameliorating the amount of pain experienced by a subject relative to that subject's pain immediately before the administration of the compounds of the present invention.

Another outcome of treatment may be preventing further pain experienced by a subject after the administration of the compounds of the present invention.

Prevention of pain refers to the administration of the compounds of the present invention to reduce the pain of a subject at risk of pain. Prevention includes, but is not limited to, the administration to a subject prior to surgery or other expected painful event. One outcome of prevention may be reducing pain in a subject at risk of pain. Another outcome of prevention may be suppressing pain in a subject at risk of pain. Another outcome of prevention may be ameliorating pain in a subject at risk of pain. Another outcome of prevention may be alleviating pain in a subject at risk of pain. Another outcome of prevention may be managing pain in a subject at risk of pain.

The terms “administration of” and or “administering a” compound should be understood to mean providing a compound of the invention or a prodrug of a compound of the invention to the individual or mammal in need of treatment.

The administration of the compound of structural formula I in order to practice the present methods of therapy is carried out by administering an effective amount of the compound of structural formula I to the mammal in need of such treatment or prophylaxis. The need for a prophylactic administration according to the methods of the present invention is determined via the use of well known risk factors. The effective amount of an individual compound is determined, in the final analysis, by the physician or veterinarian in charge of the case, but depends on factors such as the exact disease to be treated, the severity of the disease and other diseases or conditions from which the patient suffers, the chosen route of administration other drugs and treatments which the patient may concomitantly require, and other factors in the physician's judgment.

The usefulness of the present compounds in these diseases or disorders may be demonstrated in animal disease models that have been reported in the literature.

### Administration and Dose Ranges

Any suitable route of administration may be employed for providing a mammal, especially a human, with an effective dose of a compound of the present invention. For example, oral, intravenous, infusion, subcutaneous, transcutaneous, intramuscular, intradermal, transmucosal, intramucosal, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of the present invention are administered orally.

In the treatment or prevention of disorders, diseases and/or conditions which require inhibition of Naᵥ1.8 sodium ion channel activity, a suitable dosage level will generally be about 0.0001 to 500 mg per kg patient body weight per day which can be administered in single or multiple doses. In one embodiment, a suitable dosage level may be about 0.001 to 500 mg per kg patient body weight per day. In another embodiment, a suitable dosage level may be about 0.001 to about 250 mg/kg per day. In another embodiment, a suitable dosage level may be about 0.01 to about 250 mg/kg per day. In another embodiment, a suitable dosage level may be about 0.1 to about 100 mg/kg per day. In another embodiment, a suitable dosage level may be about 0.05 to 100 mg/kg per day. In another embodiment, a suitable dosage level may be about 0.1 to 50 mg/kg per day. In another embodiment, a suitable dosage level may be about 0.05 to 0.5 mg/kg per day. In another embodiment, a suitable dosage level may be about 0.5 to 5 mg/kg per day. In another embodiment, a suitable dosage level may be about 5 to 50 mg/kg per day. For oral administration, the compositions are preferably provided in the form of tablets containing 0.01 to 1000 mg of the active ingredient, particularly 0.01, 0.025, 0.05, 0.075, 0.1, 0.25, 0.5, 0.75, 1.0, 2.5, 5.0, 7.5, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient for the symptomatic adjustment of the dosage to the patient to be treated. The compounds may be administered on a regimen of 1 to 8 times per day; preferably, 1 to 4 times a day; more preferably once or twice per day. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The compounds of this invention may be used in pharmaceutical compositions comprising (a) the compound(s) or pharmaceutically acceptable salts thereof, and (b) a pharmaceutically acceptable carrier. The compounds of this invention may be used in pharmaceutical compositions that include one or more other active pharmaceutical ingredients. The compounds of this invention may also be used in pharmaceutical compositions in which the compound of the present invention or a pharmaceutically acceptable salt thereof is the only active ingredient.

The term “composition,” as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

Compounds of the present invention may be used in combination with other drugs that may also be useful in the treatment or amelioration of the diseases or conditions for which compounds of the present invention are useful. Such other drugs may be administered, by a route and in an amount commonly used therefor, contemporaneously or sequentially with a compound of the present invention. In the treatment of patients who have pain conditions, pruritic conditions and cough conditions, more than one drug is commonly administered. The compounds of this invention may generally be administered to a patient who is already taking one or more other drugs for these conditions. Often the compounds will be administered to a patient who is already being treated with one or more anti-pain compounds when the patient's pain is not adequately responding to treatment.

The combination therapy also includes therapies in which the compound of the present invention and one or more other drugs are administered on different overlapping schedules. It is also contemplated that when used in combination with one or more other active ingredients, the compound of the present invention and the other active ingredients may be used in lower doses than when each is used singly. Accordingly, the pharmaceutical compositions of the present invention include those that contain one or more other active ingredients, in addition to a compound of the present invention.

Examples of other active ingredients that may be administered in combination with a compound of the present invention, and either administered separately or in the same pharmaceutical composition, include but are not limited to:
(i) an opioid agonist;
(ii) an opioid antagonist;
(iii) a calcium channel antagonist;
(iv) a NMDA receptor agonist;
(v) a NMDA receptor antagonist;
(vi) a COX-2 selective inhibitor;
(vii) a NSAID (non-steroidal anti-inflammatory drug);
(viii) an analgesic;
(ix) a sodium channel inhibitor;
(x) an anti-NGF antibody;
(xi) a Naᵥ1.7 inhibitor;
(xii) a HCN inhibitor;
(xiii) a TRPV1 antagonist;
(xiv) a Naᵥ1.7 biological; and
(xv) a Naᵥ1.8 biological; and
   pharmaceutically acceptable salts thereof.

In another embodiment of the present invention, the pharmaceutical composition comprises:

(1) a compound of claim 1 or a pharmaceutically acceptable salt thereof;
(2) one or more compounds, or pharmaceutically acceptable salts thereof, selected from the group consisting of:
(i) an opioid agonist;
(ii) an opioid antagonist;
(iii) a calcium channel antagonist;
(iv) a NMDA receptor agonist;
(v) a NMDA receptor antagonist;
(vi) a COX-2 selective inhibitor;
(vii) a NSAID (non-steroidal anti-inflammatory drug);
(viii) an analgesic;
(ix) a sodium channel inhibitor;
(x) an anti-NGF antibody;
(xi) a Naᵥ1.7 inhibitor;
(xii) a HCN inhibitor;
(xiii) a TRPV1 antagonist;
(xiv) a Naᵥ1.7 biological; and
(xv) a Naᵥ1.8 biological; and
   pharmaceutically acceptable salts thereof; and
   (3) a pharmaceutically acceptable carrier.

A Nav 1.7 biological means a protein, including, but not limited to, antibodies, nanobodies and peptides, that inhibits the function of the Nav1.7 channel. A Nav 1.8 biological means a protein, including, but not limited to, antibodies, nanobodies and peptides, that inhibits the function of the Nav1.8 channel.

Specific compounds of use in combination with a compound of the present invention include: sodium channel inhibitors, including but not limited to, lidocaine including the lidocaine patch; tricyclic antidepressants including, but not limited to, amitriptyline; and SRI/NRI drugs, including but not limited to, duloxetine.

Suitable opioid agonists include, but are not limited to, codeine, fentanyl, hydrocodone, hydromorphone, levorphanol, meperidine, methadone, morphine, oxycodone, oxymorphone, buprenorphine, butorphanol, dezocine, nalbuphine, pentazocine, and tramadol.

Suitable opioid antagonists include, but are not limited to, naltrexone and naloxone.

Suitable calcium channel antagonists include, but are not limited to, Amlodipine, Diltiazem, Felodipine, gabapentin, Isradipine, Nicardipine, Nifedipine, Nisoldipine, pregabalin, Verapamil, and ziconitide.

Suitable NMDA receptor antagonists include, but are not limited to, ketamine, methadone, memantine, amantadine, and dextromethorphan.

Suitable COX-2 inhibitors include, but are not limited to, celecoxib, etoricoxib and parecoxib.

Suitable NSAIDs or non-steroidal anti-inflammatory drugs include, but are not limited to, aspirin, diclofenac, diflunisal, etodolac, fenoprofin, flurbiprofen, ibuprofen, indomethacin, ketoprofen, meclofenamic acid, mefenamic acid, meloxicam, naproxen, naproxen sodium, oxaprozin, piroxicam, sulindac, and tolmetin.

Suitable analgesics include, but are not limited to, acetaminophen and duloxetine.

The above combinations include combinations of a compound of the present invention not only with one other active compound, but also with two or more other active compounds. Non-limiting examples include combinations of compounds with two or more active compounds selected from: opioid agonists; opioid antagonists; calcium channel antagonists; NMDA receptor agonists; NMDA receptor antagonists; COX-2 selective inhibitors; NSAIDs (non-steroidal anti-inflammatory drugs); and an analgesic.

The compounds of the present invention, or a pharmaceutically acceptable salt thereof, may also be used in combination with spinal cord stimulation therapy and cutaneous stimulation therapy.

The present invention also provides a method for the treatment or prevention of a Naᵥ1.8 sodium ion channel activity mediated disease, disorder or condition, which method comprises administration to a patient in need of such treatment or at risk of developing a Naᵥ1.8 sodium ion channel activity mediated disease with a therapeutically effective amount of a Naᵥ1.8 sodium ion channel activity inhibitor and an amount of one or more active ingredients, such that together they give effective relief.

In a further aspect of the present invention, there is provided a pharmaceutical composition comprising a Naᵥ1.8 sodium ion channel activity inhibitor and one or more active ingredients, together with at least one pharmaceutically acceptable carrier or excipient.

Thus, according to a further aspect of the present invention there is provided the use of a Naᵥ1.8 sodium ion channel activity inhibitor and one or more active ingredients for the manufacture of a medicament for the treatment or prevention of a Naᵥ1.8 sodium ion channel activity mediated disease, disorder or condition. In a further or alternative aspect of the present invention, there is therefore provided a product comprising a Naᵥ1.8 sodium ion channel activity inhibitor and one or more active ingredients as a combined preparation for simultaneous, separate or sequential use in the treatment or prevention of a Naᵥ1.8 sodium ion channel activity mediated disease, disorder or condition. Such a combined preparation may be, for example, in the form of a twin pack.

It will be appreciated that for the treatment or prevention of pain conditions, pruritic conditions and cough conditions, a compound of the present invention may be used in conjunction with another pharmaceutical agent effective to treat that disease, disorder or condition.

The present invention also provides a method for the treatment or prevention of pain conditions, pruritic conditions and cough conditions, which method comprises administration to a patient in need of such treatment an amount of a compound of the present invention and an amount of another pharmaceutical agent effective to threat that disorder, disease or condition, such that together they give effective relief.

The present invention also provides a method for the treatment or prevention of pain conditions, pruritic conditions and cough conditions, which method comprises administration to a patient in need of such treatment an amount of a compound of the present invention and an amount of another pharmaceutical agent useful in treating that particular condition, disorder or disease, such that together they give effective relief.

The term “therapeutically effective amount” means the amount the compound of structural formula I that will elicit the biological or medical response of a cell, tissue, system, animal or human that is being sought by the researcher, veterinarian, medical doctor or other clinician, which includes alleviation of the symptoms of the disorder being treated. The novel methods of treatment of this invention are for disorders known to those skilled in the art. The term “mammal” includes humans, and companion animals such as dogs and cats.

The weight ratio of the compound of the Formula I to the second active ingredient may be varied and will depend upon the effective dose of each ingredient. Generally, an effective dose of each will be used. Thus, for example, when a compound of the Formula I is combined with a COX-2 inhibitor the weight ratio of the compound of the Formula I to the COX-2 inhibitor will generally range from about 1000:1 to about 1:1000, preferably about 200:1 to about 1:200. Combinations of a compound of the Formula I and other active ingredients will generally also be within the aforementioned range, but in each case, an effective dose of each active ingredient should be used.

### Methods of Synthesis

The following reaction schemes and Examples illustrate methods which may be employed for the synthesis of the compounds of structural formula I described in this invention. These reaction schemes and Examples are provided to illustrate the invention and are not to be construed as limiting the invention in any manner. All substituents are as defined above unless indicated otherwise. Several strategies based upon synthetic transformations known in the literature of organic synthesis may be employed for the preparation of the compounds of structural formula I. The scope of the invention is defined by the appended claims.

### Instrumentation

Reverse phase chromatography was carried out on a Gilson GX-281 equipped with a column selected from the following: Phenomenex Synergi C₁₈ (150 mm×30 mm×4 micron), YMC-Actus Pro C18 (150 mm×30 mm×5 micron), Xtimate C18 (150 mm×25 mm×5 micron), Boston Green ODS (150 mm×30 mm×5 micron), XSELECT C18 (150 mm×30 mm×5 micron), and Waters XSELECT C18 (150 mm×30 mm×5 micron). Conditions are noted for some examples.

SFC chiral resolution was carried out on a Sepiate Prep. SFC 100, Multigram II (MG II), THAR80 prep. SFC, or a Waters SFC (80, 200, or 350) using the following conditions: Chiral Method A: AD-3 column, 5-40% EtOH (0.05% DEA)/CO₂; Chiral Method B: AD-H column, 20% MeOH/CO₂; Chiral Method C: AD-H column, 50% MeOH (0.1% DEA)/CO₂; Chiral Method D: AD-H column, 10% MeOH/CO₂; Chiral Method E: AD column, 20% EtOH (0.1% NH₃·H₂O)/CO₂; Chiral Method F: AD column, 20% IPA (0.1% NH₃·H₂O)/CO₂; Chiral Method G: IG column, 20% MeOH (0.1% NH₃·H₂O)/CO₂; or Chiral Method H: AS-3 column, 5-40% MeOH (0.05% DEA)/CO₂.

LC/MS determinations were carried out on a Waters Classing Aquity system equipped with TUV and MS detectors and a Waters SQD mass spectrometer, a Shimadzu 20 UV 254 and 220 nM with Shimadzu 2010 or 2020 mass spectrometer, or an Agilent 1200 HPLC quipped with DAD/ELSD and G6110 MSD using one of the following conditions: 1) Ascentis Express C18 (3×50 mm) 2.7 μm column using mobile phase containing A: 0.05% TFA in water and B: 0.05% TFA in acetonitrile with a gradient from 90:10 (A:B) to 5:95 (A:B) over 6 min at a flow rate of 1.8 mL/min, UV detection at 210 nm; 2) Aquity BEH C18, (1.0×50 mm) 1.7 μm column using mobile phase containing A: 0.05% TFA in water and B: 0.05% TFA in acetonitrile with a gradient from 90:10 (A:B) to 5:95 (A:B) over 2 min at a flow rate of 0.3 mL/min, UV detection at 215 nm; 3) Agilent YMC J′Sphere H-80 (3×50 mm) 5 μm column using mobile phase containing A: 0.1% TFA in water and B: acetonitrile with a gradient from 95:5 (A:B) to 0:100 (A:B) over 3.6 min and 0:100 (A:B) for 0.4 min at a flow rate of 1.4 mL/min, UV detection at 254 and 220 nm and Agilent 1100 quadrupole mass spectrometer; or 4) an Agilent TC-C18 (2.1×50 mm) 5 μm column using mobile phase containing A: 0.0375% TFA in water and B: 0.01875% TFA in acetonitrile with a gradient from 90:10 (A:B) for 0.4 min to 90:10 to 0:100 (A:B) over 3 min and 10:90 (A:B) for 0.6 min at a flow rate of 0.8 mL/min, UV detection at 254 and 220 nm and Agilent 6110 quadrupole mass spectrometer.

Proton or ¹H NMR was acquired using a Varian Unity-Inova 400 MHz NMR spectrometer equipped with a Varian 400 ATB PFG 5 mm, Nalorac DBG 400-5 or a Nalorac IDG 400-5 probe, a Varian-400 MHz MR spectrometer equipped with an Auto X ID PFG Probe 5 mm, a Varian 400 MHz VNMRS spectrometer equipped with a PFG 4Nuc Probe 5 mm, or a Bruker AvanceIII 500 MHz spectrometer equipped with a PABBO Probe 5 mm in accordance with standard analytical techniques, unless specified otherwise, and results of spectral analysis are reported. Chemical shift (δ) values are reported in delta (δ) units, parts per million (ppm). Chemical shifts for ¹H NMR spectra are given relative to signals for residual non-deuterated solvent (CDCl₃ referenced at δ 7.26 ppm; DMSO d-6 referenced at δ 2.50 ppm and CD₃OD referenced at δ 3.31 ppm). Multiples are reported by the following abbreviations: s=singlet, d=doublet, t=triplet, q=quartet, dd=doublet of doublets, m=multiplet or overlap of nonequivalent resonances. Coupling constants (J) are reported in Hertz (Hz).

### Abbreviations

AIBN is azobisisobutyronitrile; Calc'd is calculated; CDI is 1,1′-carbonyldiimidazole; DCM is dichloromethane; DEA is diethanolamine; DIEA is diisopropylamine; DMA is dimethyl-acetamide; DMF is dimethylformamide; DMSO is dimethylsulfoxide; dppf is 1,1′-bis(diphenylphosphino)ferrocene; EDC is 1-ethyl-3-(3-dimethylaminopropyl)carbodiimide; EDC HCl is 1-ethyl-3-(3-dimethylaminopropyl)carbodiimide hydrochloride; Et₂O is diethyl ether; EtOAc is ethyl acetate; EtOH is ethanol; g is grams; h or hr(s) is hour(s); HATU is 1-[bis(dimethylamino)-methylene]-1H-1,2,3-triazolo[4,5-b]pyridinium-3-oxidehexafluoro-phosphate; Hex is hexanes; HMPA is hexamethylphosphoramide; HOAt is 1-Hydroxy-7-azabenzotriazole; HOBt is hydroxybenzotriazole; HPLC is high-performance liquid chromatography; IPA is isopropyl alcohol; iPrMgCl is isopropylmagnesium chloride; iPrMgCl—LiCl is isopropylmagnesium chloride lithium chloride complex; L is liter; LC/MS is liquid chromatography/mass spectrometry; LRMS is low resolution mass spectrometry; M is molar; Me is methyl; MeCN is acetonitrile; MeMgBr is methyl magnesium bromide; MeOH is methanol; mg is milligrams; mL is milliliter; mmol is millimolar; NBS is n-bromosuccinimide; NH₄OAc is ammonium acetate, NMO is 4-Methylmorpholine N-oxide; NMP is N-methyl-pyrrolidone; OEt is ethoxy; PE is petroleum ether; OiPr is isopropoxy; Pd(dppf)Cl₂ is [1,1′-bis(diphenyl-phosphino)-ferrocene]dichloropalladium(II); PE is petroleum ether; prep. is preparative; rt or RT is room temperature; SFC is Supercritical Fluid Chromatography; T3P® is 2,4,6-Tripropyl-1,3,5,2,4,6-trioxatriphosphorinane-2,4,6-trioxide; TEA is triethylamine; TFA is trifluoroacetic acid; THF is tetrahydrofuran; Ti(OEt)₄ is titanium (IV) ethoxide; Ti(OiPr)₄ is titanium (IV) isopropoxide; UV is ultraviolet.

As illustrated in Scheme A, in general, compounds of the invention can be prepared by condensation between an appropriately functionalized aldehyde A-1 and tert-butanesulfinamide, utilizing dehydrating agents such as Ti(OEt)₄ or Ti(OiPr)₄, to afford intermediate A-2. Intermediate A-2 can then be reacted with a variety of organometallic nucleophiles A-3 to give intermediate A-4 which can be deprotected under acidic conditions to give amines of formula A-5. Amine A-5 can then be brought together with lactam A-6, utilizing amide coupling conditions to deliver compounds of formula A-7. In some embodiments, a protecting group, may need to be removed throughout the course of synthesis. Aldehydes of type A-1 and organometallics of type A-3 are commercially available or may be synthesized from appropriate intermediates.

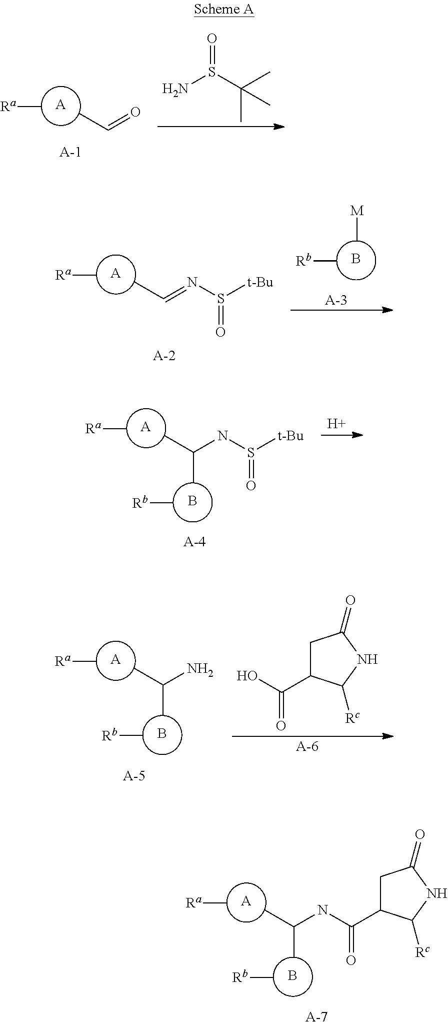

As illustrated in Scheme B, in general, compounds of the invention can be prepared by activation of appropriately functionalized carboxylic acid B-1 with either (COCl)₂ or amide coupling with amine B-2 to give intermediates of B-3. The B-3 intermediates are suitable for reaction with a variety of organometallic nucleophiles A-3 to give intermediate B-4. Intermediate B-4 can then undergo reductive amination reaction in the presence of an amine source and reductant to yield intermediates of A-5. In some cases, tert-butanesulfinamide was used as the amine source and requires deprotection (in an acidic environment) following reductive amination. Amine A-5 can then be brought together with lactam A-6, utilizing amide coupling conditions (Z═OH) or nucleophilic displacement reactions (Z═Cl) to deliver compounds of formula A-7. In some embodiments, a protecting group, may need to be removed throughout the course of synthesis. Carboxylic acid of type B-1 and organometallics of type A-3 are commercially available or may be synthesized from appropriate intermediates.

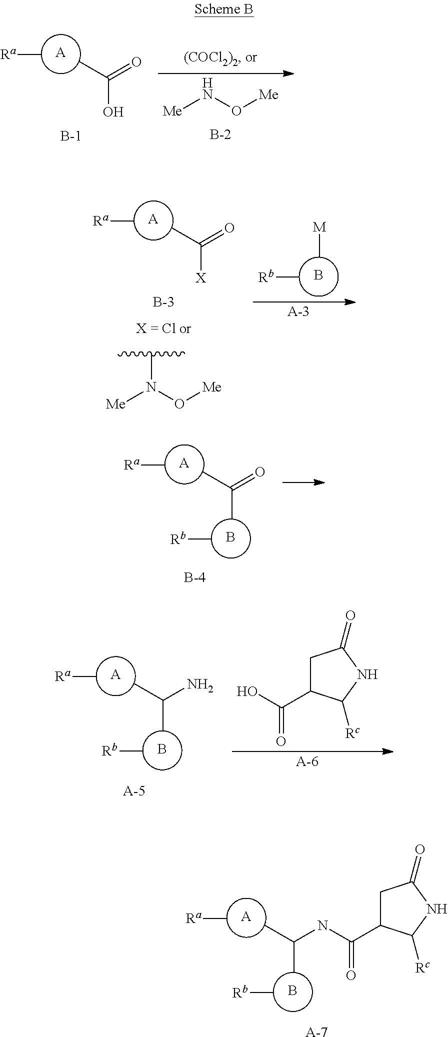

### INTERMEDIATES

### Intermediate 1

### 5-chloro-6-(trifluoromethyl)picolinaldehyde

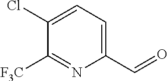 [SMILES: O=Cc1ccc(Cl)c(C(F)(F)F)n1]

Step 1: 3-chloro-2-(trifluoromethyl)-6-vinylpyridine. To a mixture of 3,6-dichloro-2-(trifluoromethyl)pyridine (1.0 g, 4.6 mmol), potassium trifluoro(vinyl)borate (0.93 g, 6.9 mmol) and K₂CO₃ (1.3 g, 9.3 mmol) in THF (15 mL) and water (3 mL) was added Pd(dppf)Cl₂ (0.17 g, 0.23 mmol). The mixture was stirred at 80° C. for 3 h. To the mixture was added water, followed by extraction with DCM. The combined organic layers were dried over Na₂SO₄. The mixture was filtered and concentrated in vacuo to give the title compound.

Step 2: 5-chloro-6-(trifluoromethyl)picolinaldehyde. A mixture of 3-chloro-2-(trifluoromethyl)-6-vinylpyridine (0.96 g crude), NMO (1.1 g, 9.3 mmol) and 0504 (2.3 mL, 0.23 mmol) in THF (10 mL) and water (5 mL) was stirred at 20° C. for 12 h. Then NaIO₄ (3.0 g, 14 mmol) was added and the mixture and stirred at 20° C. for additional 2 h. Then water was added, and the mixture was extracted with DCM. The combined organic layers were separated, dried over Na₂SO₄, filtered and concentrated in vacuo to give the title compound.

### EXAMPLES

### Examples 1A and 1B

### (S—N—((R)-(3-chloro-4-fluorophenyl)(4-chlorophenyl)methyl)-5-oxopyrrolidine-3-carboxamide and (S)—N—((S)-(3-chloro-4-fluorophenyl)(4-chlorophenyl)methyl)-5-oxopyrrolidine-3-carboxamide

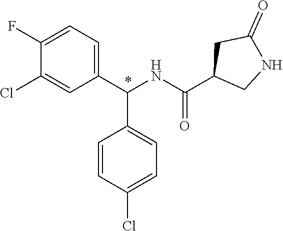 [SMILES: O=C1C[C@H](C(=O)NC(c2ccc(Cl)cc2)c2ccc(F)c(Cl)c2)CN1]

Step 1: (S—N—((R and S)-(3-chloro-4-fluorophenyl)(4-chlorophenyl)methyl)-5-oxopyrrolidine-3-carboxamide. (S)-5-oxopyrrolidine-3-carboxylic acid (0.65 g, 5.0 mmol), (3-chloro-4-fluorophenyl)(4-chlorophenyl)methanamine HCl (1.5 g, 5.0 mmol) and HATU (1.9 g, 5.0 mmol) were taken up in DMSO (16 mL) and then N-Methylmorpholine (1.6 mL, 15 mmol) was added. This solution was allowed to stir for 10 h at rt. Then mixture was purified by reverse phase HPLC (75:25 to 35:65; water (0.1% TFA):MeCN (0.1% TFA)) followed by lyophilization to give the title compound.

Step 2: (S—N—((R or S)-(3-chloro-4-fluorophenyl)(4-chlorophenyl)methyl)-5-oxopyrrolidine-3-carboxamide. (S—N—((R and S)-(3-chloro-4-fluorophenyl)(4-chlorophenyl)methyl)-5-oxopyrrolidine-3-carboxamide was separated by chiral-SFC (method A) to give title compounds: first eluted diastereomer 1A: (S—N—((R or S)-(3-chloro-4-fluorophenyl)(4-chlorophenyl)methyl)-5-oxopyrrolidine-3-carboxamide, and second eluted diastereomer 1B: (S—N—((R and S)-(3-chloro-4-fluorophenyl)(4-chlorophenyl)methyl)-5-oxopyrrolidine-3-carboxamide. Diastereomer 1A: LRMS m/z (M+H): calculated 381.1, observed 381.1. ¹H NMR (400 MHz, CD₃OD-d₄) δ 7.30-7.44 (m, 3H), 7.13-7.28 (m, 4H), 6.04-6.26 (m, 1H), 3.57-3.66 (m, 1H), 3.36-3.54 (m, 2H), 2.54 (d, J=8.4 Hz, 2H). Diastereomer 1B: LRMS m/z (M+H): calculated 381.1, observed 381.1. ¹H NMR (500 MHz, CD₃OD-d₄) δ 8.98 (br d, J=7.93 Hz, 1H), 7.31-7.44 (m, 3H), 7.14-7.28 (m, 4H), 6.05-6.24 (m, 1H), 3.55-3.66 (m, 1H), 3.36-3.52 (m, 2H), 2.43-2.62 (m, 2H).

TABLE 1
The compounds of Examples 2-13B were prepared according to a synthetic
procedure similar to the synthetic procedure for Examples 1A and 1B.

| | | | Calc’d | Observed | |
|---|---|---|---|---|---|
| Example | Structure | Name | [M + H]⁺ | [M + H]⁺ | Conditions |
| 2 | 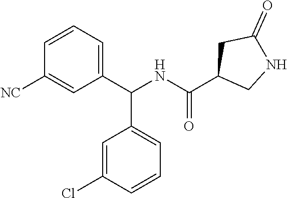 [SMILES: N#Cc1cccc(C(NC(=O)[C@@H]2CNC(=O)C2)c2cccc(Cl)c2)c1] | (3S)-N-((3- chlorophenyl)(3- cyanophenyl)methyl)- 5-oxopyrrolidine- 3-carboxamide | 354.1 | 354.1 | Not resolved |
| 3 | 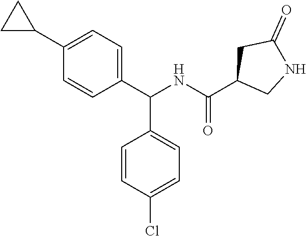 [SMILES: O=C1C[C@H](C(=O)NC(c2ccc(Cl)cc2)c2ccc(C3CC3)cc2)CN1] | (3S)-N-((4- chlorophenyl)(4- cyclopropylphenyl) methyl)-5- oxopyrrolidine-3- carboxamide | 396.1 | 369.2 | Not resolved |
| 4 | 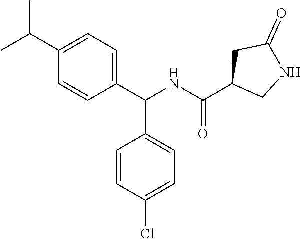 [SMILES: CC(C)c1ccc(C(NC(=O)[C@@H]2CNC(=O)C2)c2ccc(Cl)cc2)cc1] | (3S)-N-((4- chlorophenyl)(4- isopropylphenyl) methyl)-5- oxopyrrolidine-3- carboxamide | 371.2 | 371.2 | Not resolved |
| 5 | 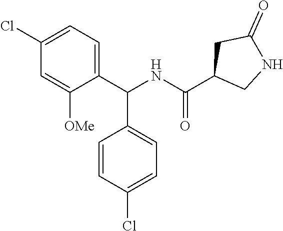 [SMILES: COc1cc(Cl)ccc1C(NC(=O)[C@@H]1CNC(=O)C1)c1ccc(Cl)cc1] | (3S)-N-((4-chloro- 2-methoxyphenyl) (4-chlorophenyl) methyl)-5- oxopyrrolidine- 3-carboxamide | 393.1 | 393.1 | Not resolved |
| 6 | 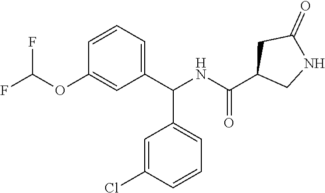 [SMILES: O=C1C[C@H](C(=O)NC(c2cccc(Cl)c2)c2cccc(OC(F)F)c2)CN1] | (3S)-N-((3- chlorophenyl)(3- (difluoromethoxy) phenyl)methyl)-5- oxopyrrolidine-3- carboxamide | 395.1 | 395.1 | Not resolved |
| 7 | 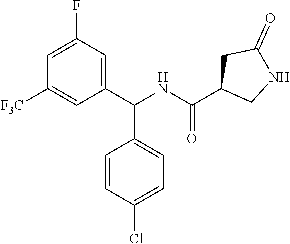 [SMILES: O=C1C[C@H](C(=O)NC(c2ccc(Cl)cc2)c2cc(F)cc(C(F)(F)F)c2)CN1] | (3S)-N-((4- chlorophenyl)(3- fluoro-5- (trifluoromethyl) phenyl)methyl)-5- oxopyrrolidine-3- carboxamide | 415.1 | 415.1 | Not resolved |
| 8 | 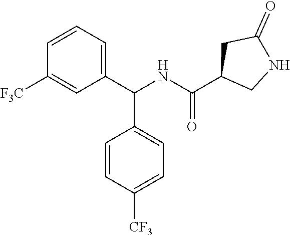 [SMILES: O=C1C[C@H](C(=O)NC(c2ccc(C(F)(F)F)cc2)c2cccc(C(F)(F)F)c2)CN1] | (3S)-5-oxo-N-((3- (trifluoromethyl) phenyl)(4- (trifluoromethyl) phenyl)methyl) pyrrolidine-3- carboxamide | 431.1 | 431.2 | Not resolved |
| 9 | 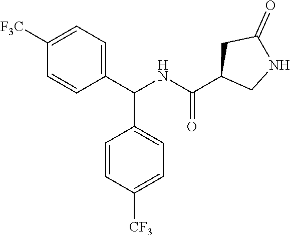 [SMILES: O=C1C[C@H](C(=O)NC(c2ccc(C(F)(F)F)cc2)c2ccc(C(F)(F)F)cc2)CN1] | (S)-N-(bis(4- (trifluoromethyl) phenyl)methyl)-5- oxopyrrolidine-3- carboxamide | 431.1 | 431.2 | Not resolved |
| 10 | 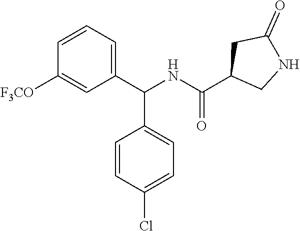 [SMILES: O=C1C[C@H](C(=O)NC(c2ccc(Cl)cc2)c2cccc(OC(F)(F)F)c2)CN1] | (3S)-N-((4- chlorophenyl)(3- (trifluoromethoxy) phenyl)methyl)-5- oxopyrrolidine-3- carboxamide | 413.1 | 413.1 | Not resolved |
| 11A | 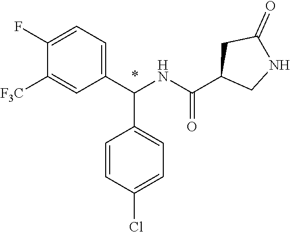 [SMILES: O=C1C[C@H](C(=O)NC(c2ccc(Cl)cc2)c2ccc(F)c(C(F)(F)F)c2)CN1] | (S)-N-((R or S)-(4- chlorophenyl)(4- fluoro-3- (trifluoromethyl) phenyl)methyl)-5- oxopyrrolidine-3- carboxamide | 415.1 | 415.3 | Chiral method B, Peak 1 |
| 11B | 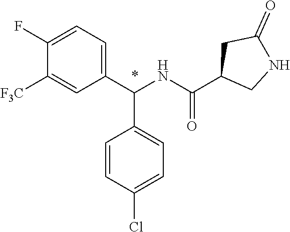 [SMILES: O=C1C[C@H](C(=O)NC(c2ccc(Cl)cc2)c2ccc(F)c(C(F)(F)F)c2)CN1] | (S)-N-((R or S)-(4- chlorophenyl)(4- fluoro-3- (trifluoromethyl) phenyl)methyl)-5- oxopyrrolidine-3- carboxamide | 415.1 | 415.3 | Chiral method B, Peak 2 |
| 12A | 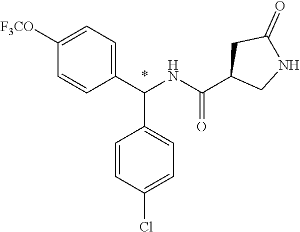 [SMILES: O=C1C[C@H](C(=O)NC(c2ccc(Cl)cc2)c2ccc(OC(F)(F)F)cc2)CN1] | (S)-N-((R or S)-(4- chlorophenyl)(4- (trifluoromethoxy) phenyl)methyl)-5- oxopyrrolidine-3- carboxamide | 413.1 | 413.3 | Chiral method B, Peak 1 |
| 12B | 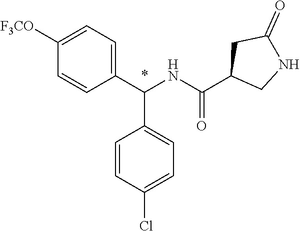 [SMILES: O=C1C[C@H](C(=O)NC(c2ccc(Cl)cc2)c2ccc(OC(F)(F)F)cc2)CN1] | (S)-N-((R or S)-(4- chlorophenyl)(4- (trifluoromethoxy) phenyl)methyl)-5- oxopyrrolidine-3- carboxamide | 413.1 | 413.3 | Chiral method B, Peak 2 |
| 13A | 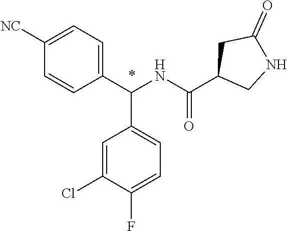 [SMILES: N#Cc1ccc(C(NC(=O)[C@@H]2CNC(=O)C2)c2ccc(F)c(Cl)c2)cc1] | (S)-N-((R or S)-(3- chloro-4- fluorophenyl)(4- cyanophenyl)methyl)- 5-oxopyrrolidine- 3-carboxamide | 372.1 | 371.9 | Chiral method C, Peak 1 |
| 13B | 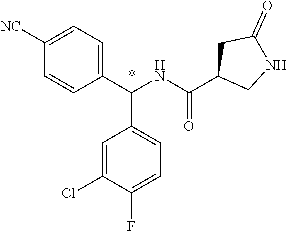 [SMILES: N#Cc1ccc(C(NC(=O)[C@@H]2CNC(=O)C2)c2ccc(F)c(Cl)c2)cc1] | (S)-N-((R or S)-(3- chloro-4- fluorophenyl)(4- cyanophenyl)methyl)- 5-oxopyrrolidine- 3-carboxamide | 372.1 | 372.3 | Chiral method C, Peak 2 |

### Examples 14A and 14B

### (S—N—((R)-(4-chloro-3-(trifluoromethyl)phenyl)(4-chlorophenyl)methyl)-5-oxopyrrolidine-3-carboxamide and (S)—N—((S)-(4-chloro-3-(trifluoromethyl)phenyl)(4-chlorophenyl)methyl)-5-oxopyrrolidine-3-carboxamide

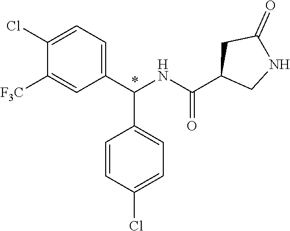 [SMILES: O=C1C[C@H](C(=O)NC(c2ccc(Cl)cc2)c2ccc(Cl)c(C(F)(F)F)c2)CN1]

Step 1: (S—N—((R and S)-(4-chloro-3-(trifluoromethyl)phenyl)(4-chlorophenyl)methyl)-5-oxopyrrolidine-3-carboxamide. (S)-5-oxopyrrolidine-3-carboxylic acid (78 mg, 0.60 mmol), (3-chloro-4-fluorophenyl)(4-chlorophenyl)methanamine HCl (0.26 mg, 0.72 mmol) and HATU (0.27 mg, 0.72 mmol) were taken up in DMSO (1.2 mL) and then N-Methylmorpholine (0.24 μl, 2.2 mmol) was added. This solution was allowed to stir for 10 h at rt. The mixture was purified by mass directed reverse phase HPLC to give the title compound.

Step 2: (S—N—((R or S)-(4-chloro-3-(trifluoromethyl)phenyl)(4-chlorophenyl)methyl)-5-oxopyrrolidine-3-carboxamide. (S—N—((R and S)-(4-chloro-3-(trifluoromethyl)phenyl)(4-chlorophenyl)methyl)-5-oxopyrrolidine-3-carboxamide was separated by chiral-SFC (AD-H column, 25% MeOH/CO₂) to give title compounds: first eluted diastereomer 14A (S—N—((R or S)-(4-chloro-3-(trifluoromethyl)phenyl)(4-chlorophenyl)methyl)-5-oxopyrrolidine-3-carboxamide, and second eluted diastereomer 14B (S—N—((R or S)-(4-chloro-3-(trifluoro-methyl)phenyl)(4-chlorophenyl)methyl)-5-oxopyrrolidine-3-carboxamide. Diastereomer 14A: LRMS m/z (M+H): calculated 431.1, observed 431.2. ¹H NMR (500 MHz, DMSO-d₆) δ 9.00 (d, J=8.1 Hz, 1H), 7.79 (s, 1H), 7.72 (d, J=8.3 Hz, 1H), 7.64-7.55 (m, 2H), 7.43 (d, J=8.4 Hz, 2H), 7.31 (d, J=8.3 Hz, 2H), 6.25 (d, J=8.1 Hz, 1H), 3.45 (t, J=9.0 Hz, 1H), 3.32-3.25 (m, 1H), 3.20 (dd, J=9.1, 6.2 Hz, 1H), 2.31 (p, J=9.0, 8.2 Hz, 2H). Diastereomer 14B: LRMS m/z (M+H): calculated 431.1, observed 431.2. ¹H NMR (500 MHz, DMSO-d₆) δ 9.00 (d, J=8.2 Hz, 1H), 7.80-7.76 (m, 1H), 7.73 (d, J=8.3 Hz, 1H), 7.64-7.56 (m, 2H), 7.43 (d, J=8.4 Hz, 2H), 7.32 (d, J=8.4 Hz, 2H), 6.25 (d, J=8.2 Hz, 1H), 3.45 (t, J=8.8 Hz, 1H), 3.32-3.27 (m, 1H), 3.27-3.20 (m, 1H), 2.34 (dd, J=16.6, 9.3 Hz, 1H), 2.27 (dd, J=16.6, 7.4 Hz, 1H).

### Example 15

### (S—N—((R or S)-(3-chloro-2,4-difluorophenyl)(5-chloro-6-(trifluoromethyl)pyridin-3-yl)methyl)-5-oxopyrrolidine-3-carboxamide

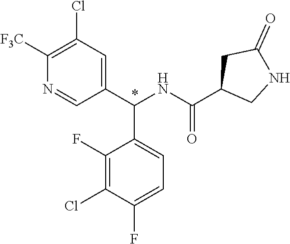 [SMILES: O=C1C[C@H](C(=O)NC(c2cnc(C(F)(F)F)c(Cl)c2)c2ccc(F)c(Cl)c2F)CN1]

Step 1: (3-chloro-2,4-difluorophenyl)(5-chloro-6-(trifluoromethyl)pyridin-3-yl)methanone. To a solution of 5-chloro-6-(trifluoromethyl)nicotinic acid (1.0 g, 4.4 mmol) in DCM (25 mL) at 0° C. was added (COCl)₂ (3.3 mL, 6.6 mmol, 2.0 M in DCM) and one drop of DMF. The mixture was warmed to rt and stirred for 4 h. Then the mixture was concentrated in vacuo before being dissolved in THF (8 mL; Solution A). In a different flask, 2-chloro-1,3-difluoro-4-iodobenzene (1.8 g, 6.6 mmol) was dissolved in THF (15 mL) and cooled to −20° C., followed by the addition of iPrMgCl—LiCl complex (5.1 mL, 6.6 mmol, 1.3 M in THF). The mixture was stirred at −20° C. for 2 h, then warmed to 0° C. and then CuCN (0.68 g, 7.5 mmol) was added. The mixture was stirred at 0° C. for 30 min, followed by the addition of Solution A. The mixture was stirred at 0° C. for 2 h, then warmed to rt and stirred for 1 h. The reaction was then quenched by addition of saturated NH₄Cl and extracted with EtOAc before being filtered through a pad of the Celite®. The separated organic layer was dried over Na₂SO₄, filtered and concentrated in vacuo. The resulting residue was purified by silica gel chromatography (0-40% EtOAc:hex) to give the title compound.

Step 2: (R)—N-((3-chloro-2,4-difluorophenyl)(5-chloro-6-(trifluoromethyl)pyridin yl)methylene)-2-methylpropane-2-sulfinamide. A microwave tube was charged with (3-chloro-2,4-difluorophenyl)(5-chloro-6-(trifluoromethyl)pyridin-3-yl)methanone (1.2 g, 3.5 mmol), (R)-2-methyl-2-propanesulfinamide (0.64 g, 5.3 mmol) and Ti(OEt)₄ (6.4 mL, 7.0 mmol). The mixture was heated via microwave irradiation at 105° C. for 1 h. Then water and EtOAc were added and the mixture was stirred for 10 min, followed by filtration through a pad of the Celite® to remove the solid. The separated organic layer was dried over Na₂SO₄, filtered and concentrated in vacuo to give the title compound.

Step 3: (R)—N-((3-chloro-2,4-difluorophenyl)(5-chloro-6-(trifluoromethyl)pyridin-3-yl)methyl)-2-methylpropane-2-sulfinamide. To a solution of (R)—N-((3-chloro-2,4-difluorophenyl)(5-chloro-6-(trifluoromethyl)pyridin-3-yl)methylene)-2-methylpropane-2-sulfinamide (1.6 g, 3.5 mmol) in THF (20 mL) and water (0.4 mL) at −78° C. was added NaBH₄ (0.40 g, 10 mmol). The mixture was stirred at −78° C. for 3 h, then gradually warmed to RT and stirred overnight. The reaction was quenched with water and extracted with EtOAc. The separated organic layer was dried over Na₂SO₄, filtered and concentrated in vacuo. The resulting residue was purified by silica gel chromatography (0-25% EtOAc:hexane), followed by separation by chiral-SFC (method D) to give the title compound (peak 1).

Step 4: (3-chloro-2,4-difluorophenyl)(5-chloro-6-(trifluoromethyl)pyridin-3-yl)methanamine, HCl. (R)—N-((3-chloro-2,4-difluorophenyl)(5-chloro-6-(trifluoromethyl)pyridin-3-yl)methyl)-2-methylpropane-2-sulfinamide (0.79 g, 1.7 mmol) was dissolved in DCM (1 mL) and MeOH (0.5 mL). The mixture was cooled to 0° C. and then HCl (1.0 mL, 4.0 mmol, 4 N in 1,4-dioxane) was added. The resulting mixture was stirred at 0° C. for 2 h and then concentrated in vacuo. The resulting residual solid was washed with Et₂O and filtered to give the title compound.

Step 5: (S—N—((R or S)-(3-chloro-2,4-difluorophenyl)(5-chloro-6-(trifluoromethyl)pyridin-3-yl)methyl)-5-oxopyrrolidine-3-carboxamide. To a solution of (3-chloro-2,4-difluorophenyl)(5-chloro-6-(trifluoromethyl)pyridin-3-yl)methanamine HCl (85 mg, 0.22 mmol) in pyridine (3 mL) was added (S)-5-oxopyrrolidine-3-carboxylic acid (42 mg, 0.32 mmol) and EDC HCl (83 mg, 0.43 mmol). The mixture was heated to 70° C. and stirred overnight. Then the mixture was concentrated in vacuo. The resulting residue was purified by silica gel chromatography (0-4% MeOH:DCM) to give the title compound. LRMS m/z (M+H): calculated 468.0, observed 468.4. ¹H NMR (500 MHz, Chloroform-d) δ 8.48 (s, 1H), 7.71 (s, 1H), 7.45 (s, 1H), 7.25-7.18 (m, 1H), 7.04 (t, J=8.2 Hz, 1H), 6.48 (d, J=8.0 Hz, 1H), 6.27 (s, 1H), 3.68-3.51 (m, 2H), 3.35 (p, J=8.0 Hz, 1H), 2.63 (dd, J=16.7, 7.8 Hz, 1H), 2.52 (dd, J=17.0, 9.2 Hz, 1H).

### Example 16

### (S—N—((R or S)-(3-chloro-2,4-difluorophenyl)((trans)-3-(trifluoromethyl)cyclobutyl)methyl)-5-oxopyrrolidine-3-carboxamide

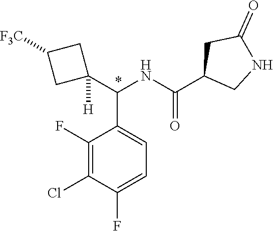 [SMILES: O=C1C[C@H](C(=O)NC(c2ccc(F)c(Cl)c2F)[C@H]2C[C@H](C(F)(F)F)C2)CN1]

Step 1: (3-chloro-2,4-difluorophenyl)(trans-3-(trifluoromethyl)cyclobutyl)methanone. To a solution of trans-3-(trifluoromethyl)cyclobutane-1-carboxylic acid (1.0 g, 6.0 mmol) in DCM (15 mL) at 0° C. was added (COCl)₂ (3.6 mL, 7.1 mmol, 2.0 M in DCM) and one drop of DMF. The mixture was warmed to rt and stirred at rt for 4 hours. Then the mixture was concentrated in vacuo. The resulting residue was dissolved in THF (6 mL; Solution A). In a separate flask, 2-chloro-1,3-difluoro-4-iodobenzene (2.4 g, 8.9 mmol) was dissolved in THF (20 mL), cooled to −20° C., followed by the addition of iPrMgCl—LiCl complex (6.9 mL, 8.9 mmol, 1.3 M in THF). The mixture was stirred at −20° C. for 2 hours, warmed to 0° C., then CuCN (1.1 g, 12 mmol) was added, and the mixture was stirred at 0° C. for 30 minutes. Solution A was added to the mixture, and the reaction was kept at 0° C. for 2 hours, and then warmed to rt for 1 hour. The mixture was partitioned between EtOAc and saturated NH₄C₁, and filtered through a pad of the Celite®. The separated organic layer was dried over Na₂SO₄, filtered and concentrated in vacuo to give the title compound.

Step 2: (R)—N-((3-chloro-2,4-difluorophenyl)(trans-3-(trifluoromethyl)cyclobutyl)methylene)-2-methylpropane-2-sulfinamide. A microwave tube was charged with (3-chloro-2,4-difluorophenyl)(trans-3-(trifluoromethyl) cyclobutyl)methanone (1.7 g, 5.7 mmol), (R)-2-methylpropane-2-sulfinamide (1.0 g, 8.5 mmol) and Ti(OEt)₄ (10 mL, 11 mmol). The mixture was microwaved at 105° C. for 1 hour and then cooled to rt. The reaction mixture was poured into water and EtOAc, stirred for 10 minutes, and then filtered through a pad of the Celite® to remove the solid. The separated organic layer was dried over Na₂SO₄, filtered and concentrated in vacuo to give the title compound.

Step 3: (R)—N-((3-chloro-2,4-difluorophenyl)(trans-3-(trifluoromethyl)cyclobutyl)methyl)-2-methylpropane-2-sulfinamide. To a solution of (R)—N-((3-chloro-2,4-difluorophenyl)(trans-3-(trifluoromethyl)cyclobutyl)methylene)-2-methylpropane-2-sulfinamide (2.2 g, 5.5 mmol) in THF (10 mL) and MeOH (2 mL) at 0° C. was added NaBH₄ (0.21 g, 5.5 mmol). The mixture was stirred at 0° C. for 1 h, then warmed to rt for 1 h, and partitioned between EtOAc and saturated NaHCO₃. The organic layer was separated, dried over Na₂SO₄, filtered and concentrated in vacuo. The resulting residue was purified by silica gel chromatography (0-40% EtOAc:hexane) to give a mixture, which was separated by chiral-SFC (method D) to give the title compound (first eluted isomer).

Step 4: (3-chloro-2,4-difluorophenyl)(trans-3-(trifluoromethyl)cyclobutyl)methanamine hydrochloride. (R)—N-((3-chloro-2,4-difluorophenyl)(trans-3-(trifluoromethyl)cyclobutyl) methyl)-2-methyl propane-2-sulfinamide (first eluted isomer; 0.12 g, 0.31 mmol) was dissolved in DCM (1 mL). The mixture was cooled to 0° C. and HCl (1.0 mL, 4.0 mmol, 4.0 M in 1,4-dioxane) was added. Then the mixture was stirred at 0° C. for 2 h and concentrated in vacuo. The resulting residue was washed with Et₂O and filtered to give the title compound.

Step 5: (S—N—((R or S)-(3-chloro-2,4-difluorophenyl)((trans)-3-(trifluoromethyl)cyclobutyl)-methyl)-5-oxopyrrolidine-3-carboxamide. (S)-5-oxopyrrolidine-3-carboxylic acid (14 g, 0.11 mmol), (3-chloro-2,4-difluorophenyl)((trans)-3-(trifluoromethyl)cyclobutyl)methanamine hydrochloride (30 mg, 0.089 mmol) and DIEA (47 μl, 0.27 mmol) were combined in DMF (0.36 mL). Then HATU (42 mg, 0.11 mmol) was added, and the mixture was stirred overnight at rt. The reaction was diluted with water and extracted with EtOAc. The combined organic layer was washed with saturated NaHCO₃, brine, dried over MgSO₄, filtered and concentrated in vacuo. The resulting residue was purified by reverse phase HPLC (95:5 to 5:95; water (0.1% TFA):MeCN (0.1% TFA), followed by lyophilization to give the title compound. LRMS m/z (M+H): calculated 411.1, observed 411.1. ¹H NMR (500 MHz, DMSO-d₆) δ 8.50 (d, J=7.6 Hz, 1H), 7.53 (s, 1H), 7.45-7.27 (m, 2H), 5.09 (t, J=9.0 Hz, 1H), 3.36 (t, J=9.0 Hz, 1H), 3.24-3.03 (m, 3H), 2.80-2.69 (m, 1H), 2.38-2.23 (m, 2H), 2.23-2.08 (m, 2H), 2.02-1.85 (m, 2H).

### Example 17

### (S—N—((R or S)-(3-chloro-4-fluorophenyl)(4-(trifluoromethyl)phenyl)methyl)-5-oxopyrrolidine-3-carboxamide

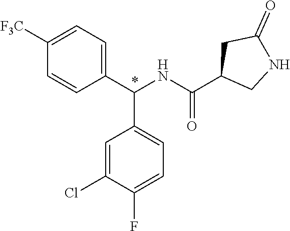 [SMILES: O=C1C[C@H](C(=O)NC(c2ccc(C(F)(F)F)cc2)c2ccc(F)c(Cl)c2)CN1]

Step 1: (S)-2-methyl-N-(4-(trifluoromethyl)benzylidene)propane-2-sulfinamide. 4-(trifluoromethyl)benzaldehyde (5.0 g, 29 mmol) and (S)-2-methylpropane-2-sulfinamide (5.2 g, 43 mmol) were taken up in THF (100 mL) and then Ti(OEt)₄ (20 g, 86 mmol) was added. The mixture was allowed to stir for 2 h, then diluted with brine, filtered through sand and extracted with EtOAc. The combined organic layers were washed with saturated NH₄C₁, brine, dried over Na₂SO₄, filtered and concentrated in vacuo. The resulting residue was purified by silica gel chromatography (10% EtOAc:PE) to give the title compound.

Step 2: (S)—N-((3-chloro-4-fluorophenyl)(4-(trifluoromethyl)phenyl)methyl)-2-methylpropane-2-sulfinamide. (S)-2-methyl-N-(4-(trifluoromethyl)benzylidene)propane-2-sulfinamide (1.6 g, 4.6 mmol) was taken up in THF (5 mL) and cooled to −40° C. To this solution was slowly added (3-chloro-4-fluorophenyl)magnesium bromide (28 mL, 14 mmol). The mixture was allowed to stir at −40° C. for 1 h and then stirred at 20° C. for 16 h before being quenched with saturated NH₄C₁. This mixture was then filtered through Celite®, eluting with EtOAc and then concentrated in vacuo. The resulting residue was purified by reverse phase HPLC (73:27 to 43:57; water (0.1% TFA):MeCN (0.1% TFA)) followed by lyophilization to give the title compound.

Step 3: (S—N—((R or S)-(3-chloro-4-fluorophenyl)(4-(trifluoromethyl)phenyl)methyl)-2-methylpropane-2-sulfinamide. (S)—N-((3-chloro-4-fluorophenyl)(4-(trifluoromethyl)phenyl)-methyl)-2-methylpropane-2-sulfinamide (1.6 g, 3.9 mmol) was separated by chiral-SFC (method A) to give the title compound (Peak 1).

Step 4: (R or S)-(3-chloro-4-fluorophenyl)(4-(trifluoromethyl)phenyl)methanamine hydrochloride. A solution of (S—N—((R or S)-(3-chloro-4-fluorophenyl)(4-(trifluoromethyl)-phenyl)methyl)-2-methylpropane-2-sulfinamide (0.90 g, 2.2 mmol) in HCl (20 mL, 80 mmol, 4 N in EtOAc) was stirred at 15° C. for 2 h. Then the mixture was concentrated in vacuo to give the title compound.

Step 5: (S—N—((R or S)-(3-chloro-4-fluorophenyl)(4-(trifluoromethyl)phenyl)methyl)-5-oxopyrrolidine-3-carboxamide. To a solution of (S)-5-oxopyrrolidine-3-carboxylic acid (50 mg, 0.39 mmol), DIEA (0.20 mL, 1.2 mmol) and (R or S)-(3-chloro-4-fluorophenyl)(4-(trifluoro-methyl)phenyl)methanamine hydrochloride (0.16 g, 0.46 mmol) in DMF (2 mL) was added T3P® (0.49 g, 0.78 mmol, 50% in DMF) at 15° C. The resulting mixture was stirred at 15° C. for 16 h. Then the reaction mixture was purified by reverse phase HPLC (73:27 to 43:57; water (0.1% TFA):MeCN (0.1% TFA)), followed by lyophilization to give the title compound. LRMS m/z (M+H): calculated 415.1, observed 415.2. ¹H NMR (400 MHz, CD₃OD-d₄) δ 7.68 (d, J=8.4 Hz, 2H), 7.46 (d, J=8.0 Hz, 2H), 7.34-7.41 (m, 1H), 7.17-7.30 (m, 2H), 6.26 (s, 1H), 3.57-3.68 (m, 1H), 3.36-3.53 (m, 2H), 2.44-2.61 (m, 2H).

TABLE 2
The compounds of Examples 18-20B were prepared according to a synthetic
procedure similar to the synthetic procedure for Example 17.

| | | | Calc’d | Observed | |
|---|---|---|---|---|---|
| Example | Structure | Name | [M + H]⁺ | [M + H]⁺ | Conditions |
| 18 | 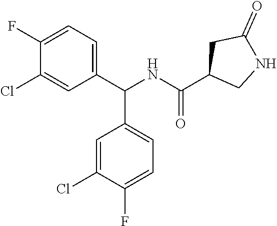 [SMILES: O=C1C[C@H](C(=O)NC(c2ccc(F)c(Cl)c2)c2ccc(F)c(Cl)c2)CN1] | (S)-N-(bis(3-chloro- 4-fluorophenyl) methyl)-5- oxopyrrolidine- 3-carboxamide | 399.0 | 399.1 | Meso compound |
| 19A | 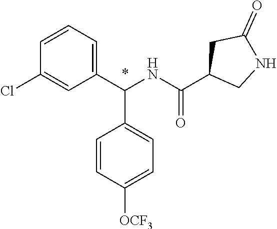 [SMILES: O=C1C[C@H](C(=O)NC(c2ccc(OC(F)(F)F)cc2)c2cccc(Cl)c2)CN1] | (S)-N-((R or S)-(3- chlorophenyl)(4- (trifluoromethoxy) phenyl)methyl)-5- oxopyrrolidine-3- carboxamide | 413.1 | 413.1 | Step 6: Chiral method E, Peak 1 |
| 19B | 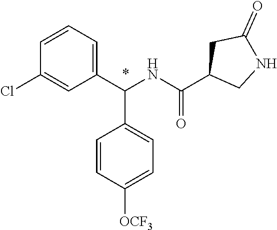 [SMILES: O=C1C[C@H](C(=O)NC(c2ccc(OC(F)(F)F)cc2)c2cccc(Cl)c2)CN1] | (S)-N-((R or S)-(3- chlorophenyl)(4- (trifluoromethoxy) phenyl)methyl)-5- oxopyrrolidine-3- carboxamide | 413.1 | 413.1 | Step 6: Chiral method E, Peak 2 |
| 20A | 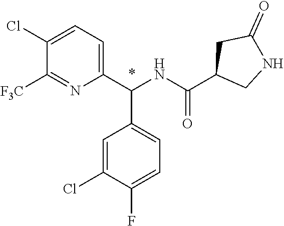 [SMILES: O=C1C[C@H](C(=O)NC(c2ccc(F)c(Cl)c2)c2ccc(Cl)c(C(F)(F)F)n2)CN1] | (S)-N-((R or S)-(3- chloro-4- fluorophenyl)(5- chloro-6- (trifluoromethyl)- pyridin-2- yl)methyl)-5- oxopyrrolidine-3- carboxamide | 450.0 | 450.0 | Step 6: Chiral method F, Peak 1 |
| 20B | 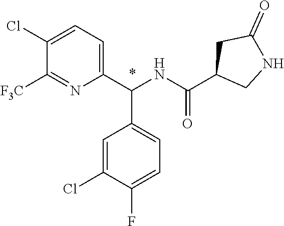 [SMILES: O=C1C[C@H](C(=O)NC(c2ccc(F)c(Cl)c2)c2ccc(Cl)c(C(F)(F)F)n2)CN1] | (S)-N-((R or S)-(3- chloro-4- fluorophenyl)(5- chloro-6- (trifluoromethyl)- pyridin-2- yl)methyl)-5- oxopyrrolidine-3- carboxamide | 450.0 | 450.1 | Step 6: Chiral method F, Peak 2 |

### Examples 21A and 21B

### (S—N—((R)-(3-chloro-2,4-difluorophenyl)(3,3-dimethylcyclobutyl)methyl)-5-oxopyrrolidine-3-carboxamide and (S)—N—((S)-(3-chloro-2,4-difluorophenyl)(3,3-dimethylcyclobutyl)methyl)-5-oxopyrrolidine-3-carboxamide

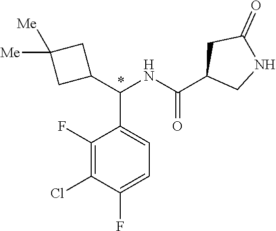 [SMILES: CC1(C)CC(C(NC(=O)[C@@H]2CNC(=O)C2)c2ccc(F)c(Cl)c2F)C1]

Step 1: N-methoxy-N,3,3-trimethylcyclobutane-1-carboxamide. To a solution of CDI (2.3 g, 14 mmol) in DCM (25 mL) was added 3,3-dimethylcyclobutane-1-carboxylic acid (0.90 g, 7.0 mmol) at 20° C. for 1 h. Then DIEA (3.7 mL, 21 mmol) and N,O-dimethyl hydroxylamine hydrochloride (0.82 g, 8.4 mmol) was added, and the resulting mixture was stirred at 20° C. for another 2 h. The reaction was quenched with water and extracted with DCM. The combined organic fractions were washed with brine, dried over Na₂SO₄, filtered and concentrated in vacuo. The crude product was purified by silica gel chromatography (15% EtOAc:PE) to give the title compound.

Step 2: (3-chloro-2,4-difluorophenyl)(3,3-dimethylcyclobutyl)methanone. To a solution of 1-bromo-3-chloro-2,4-difluorobenzene (2.2 g, 9.6 mmol) in THF (4 mL) was added iPrMgCl (4.4 mL, 8.8 mmol) at 0° C. for 2 h, then a mixture of N-methoxy-N,3,3-trimethylcyclobutane-1-carboxamide (0.50 g, 2.9 mmol) in THF (4 mL) was added. The reaction mixture was stirred at 0° C. for 12 h, then quenched with saturated NH₄C₁ and extracted with EtOAc. The combined organic layers were washed with brine, dried over Na₂SO₄, filtered and concentrated in vacuo. The crude product was then purified by silica gel chromatography (14% EtOAc:PE) to give the title compound.

Step 3: (3-chloro-2,4-difluorophenyl)(3,3-dimethylcyclobutyl)methanamine. NH₄OAc (1.8 g, 23 mmol) and NaBH₃CN (0.15 g, 2.3 mmol) were added to a solution of (3-chloro-2,4-difluorophenyl)(3,3-dimethylcyclobutyl)methanone (0.40 g, 1.5 mmol) in EtOH (4 mL) in a 20 mL microwave vial. The mixture was stirred and heated at 130° C. for 15 min via microwave irradiation. The reaction mixture was concentrated to remove most of the EtOH, then treated with 2 N NaOH until pH>10, and extracted with EtOAc. The organic layer was separated, dried over Na₂SO₄, filtered, and concentrated in vacuo to give the title compound.

Step 4: (S—N—((R and S)-(3-chloro-2,4-difluorophenyl)(3,3-dimethylcyclobutyl)methyl)-5-oxopyrrolidine-3-carboxamide. To a mixture of (3-chloro-2,4-difluorophenyl)(3,3-dimethylcyclobutyl)methanamine (0.10 g, 0.38 mmol), (S)-5-oxopyrrolidine-3-carboxylic acid (60 mg, 0.46 mmol) and DIEA (0.20 mL, 1.2 mmol) in DMF (1.5 mL) was added T3P® (0.49 g, 0.77 mmol) at 0° C. The resulting mixture was stirred at 15° C. for 1 h. The resulting residue was purified by reverse phase HPLC (52:48 to 32:68; water (0.1% TFA):MeCN (0.1% TFA)), followed by lyophilization to give the title compound.

Step 5: (S—N—((R or S)-(3-chloro-2,4-difluorophenyl)(3,3-dimethylcyclobutyl)methyl)-5-oxopyrrolidine-3-carboxamide. (S—N—((R and S)-(3-chloro-2,4-difluorophenyl)(3,3-dimethylcyclobutyl)methyl)-5-oxopyrrolidine-3-carboxamide was separated by chiral-SFC (method G) to give the title compounds: first eluted diastereomer 21A (S—N—((R or S)-(3-chloro-2,4-difluorophenyl)(3,3-dimethylcyclobutyl)methyl)-5-oxopyrrolidine-3-carboxamide, and second eluted diastereomer 21B (S—N—((R or S)-(3-chloro-2,4-difluorophenyl)(3,3-dimethylcyclobutyl)methyl)-5-oxopyrrolidine-3-carboxamide. Diastereomer 21A: LRMS m/z (M+H): calculated 371.1, observed 371.1. ¹H NMR (500 MHz, CD₃OD) δ 7.12-7.23 (m, 1H), 6.97-7.00 (m, 1H), 4.91 (dd, J=8.0, 10.5 Hz, 1H), 3.44-3.52 (m, 1H), 3.36 (dd, J=6.5, 10.0 Hz, 1H), 3.34-3.38 (m, 1H), 2.53-2.26 (m, 1H), 2.24-2.41 (m, 2H), 1.83-1.88 (m, 1H), 1.45-1.59 (m, 2H), 1.35-1.44 (m, 1H), 1.04 (s, 3H), 0.98 (s, 3H). Diastereomer 21B: LRMS m/z (M+H): calculated 371.1, observed 371.1. ¹H NMR (500 MHz, CD₃OD) δ 7.22-7.37 (m, 1H), 7.10-7.13 (m 1H), 5.03 (d, J=10.5 Hz, 1H), 3.47-3.60 (m, 1H), 3.34-3.38 (m, 2H), 2.66-2.69 (m 1H), 2.50-2.58 (m, 2H), 1.96-2.00 (m, 1H), 1.58-1.72 (m, 2H), 1.48-1.57 (m, 1H), 1.17 (s, 3H), 1.11 (s, 3H).

### Example 22

### (3S)—N-((3-chloro-4-fluorophenyl)(3-cyano-4-fluorophenyl)methyl)-5-oxopyrrolidine-3-carboxamide

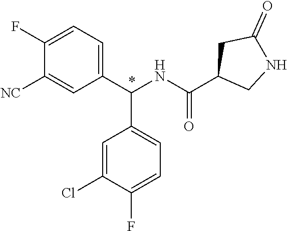 [SMILES: N#Cc1cc(C(NC(=O)[C@@H]2CNC(=O)C2)c2ccc(F)c(Cl)c2)ccc1F]

Step 1: N-(4-cyano-3-fluorobenzylidene)-2-methylpropane-2-sulfinamide. To flask containing 2-fluoro-4-formylbenzonitrile (4.3 g, 29 mmol) and 2-methylpropane-2-sulfinamide (3.9 g, 32 mmol) in THF (75 mL) was added Ti(OiPr)₄ (18 mL, 61 mmol) at rt. The reaction was quenched with saturated NH₄Cl and extracted with EtOAc. The combined organic layers were washed with saturated NH₄Cl, NaHCO₃, water, and then brine. The organic layer was dried over Na₂SO₄, filtered and concentrated in vacuo. The resulting residue was purified by silica gel chromatography (0-60% EtOAc:hex) to give the title compound.

Step 2: N-((3-chloro-4-fluorophenyl)(3-cyano-4-fluorophenyl)methyl)-2-methylpropane sulfinamide. To a vial containing N-(4-cyano-3-fluorobenzylidene)-2-methylpropane-2-sulfinamide (1.3 g, 5.2 mmol) was added THF (20 mL) under an atmosphere of nitrogen, then the mixture was cooled to 0° C., and (3-chloro-4-fluorophenyl)magnesium bromide (47 mL, 23 mmol) was added. The mixture was stirred at 0° C. for 10 min, then warmed to rt, and quenched with saturated NaHCO₃ and EtOAc with stirring for an additional 20 minutes. Solid Celite® was added, and the mixture was stirred for 10 min, and then filtered through Celitet. The filtrate was concentrated in vacuo. The resulting residue was purified by reverse phase HPLC (70:30 to 0:100; water (0.1% TFA):MeCN (0.1% TFA)), followed by lyophilization to give the title compound.

Step 3: 5-(amino(3-chloro-4-fluorophenyl)methyl)-2-fluorobenzonitrile hydrochloride. To a flask containing N-((3-chloro-4-fluorophenyl)(3-cyano-4-fluorophenyl)methyl)-2-methylpropane-2-sulfinamide (1.1 g, 2.8 mmol) was added DCM (5 mL) and MeOH (5 mL). The mixture was further diluted with EtOAc (20 mL). Then HCl gas was bubbled through the solution for 2 minutes until saturated. The reaction was stirred at room temperature for 2 h. Then the mixture was concentrated in vacuo to give the title compound.

Step 4: (3S)—N-((3-chloro-4-fluorophenyl)(3-cyano-4-fluorophenyl)methyl)-5-oxopyrrolidine-3-carboxamide. To a vial containing 5-(amino(3-chloro-4-fluorophenyl)methyl)-2-fluorobenzonitrile hydrochloride (61 mg, 0.19 mmol) was added (S)-5-oxopyrrolidine-3-carboxylic acid (30 mg, 0.23 mmol), EDC (43 mg, 0.22 mmol), HOBT (36 mg, 0.27 mmol), followed by DMF (1 mL) and DIEA (50 μL, 0.29 mmol). The reaction mixture was then diluted with water and purified by mass directed reverse phase HPLC to give the title compound. LRMS m/z (M+Na): calculated 412.1, observed 412.1. ¹H NMR (500 MHz, DMSO-d₆) δ 8.98 (d, J=8.0 Hz, 1H), 7.85 (d, J=5.8 Hz, 1H), 7.77-7.65 (m, 1H), 7.60 (s, 1H), 7.51 (t, J=9.0 Hz, 2H), 7.40 (t, J=8.9 Hz, 1H), 7.36-7.26 (m, 1H), 6.18 (d, J=8.0 Hz, 1H), 3.22 (dt, J=9.9, 5.2 Hz, 1H), 2.40-2.23 (m, 2H).

TABLE 3
The compounds of Examples 23-26 were prepared according to a synthetic procedure
similar to the synthetic procedure for Example 22.

| | | | Calc’d | Observed |
|---|---|---|---|---|
| Example | Structure | Name | [M + H]⁺ | [M + H]⁺ |
| 23 | 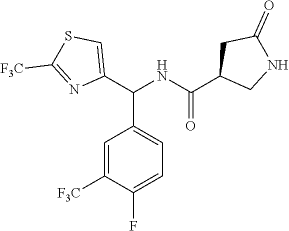 [SMILES: O=C1C[C@H](C(=O)NC(c2ccc(F)c(C(F)(F)F)c2)c2csc(C(F)(F)F)n2)CN1] | (3S)-N-((4-fluoro-3- (trifluoromethyl) phenyl)(2- (trifluoromethyl) thiazol-4-yl)methyl)- 5-oxopyrrolidine-3- carboxamide | 456.1 | 456.1 |
| 24 | 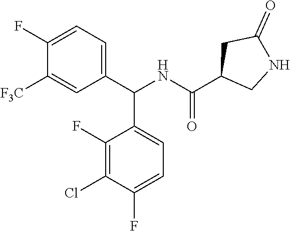 [SMILES: O=C1C[C@H](C(=O)NC(c2ccc(F)c(C(F)(F)F)c2)c2ccc(F)c(Cl)c2F)CN1] | (3S)-N-((3-chloro- 2,4- difluorophenyl)(4- fluoro-3- (trifluoromethyl) phenyl)methyl)-5- oxopyrrolidine-3- carboxamide | 451.1 | 451.1 |
| 25 | 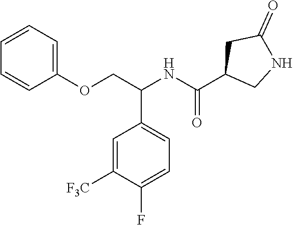 [SMILES: O=C1C[C@H](C(=O)NC(COc2ccccc2)c2ccc(F)c(C(F)(F)F)c2)CN1] | (3S)-N-(1-(4-fluoro- 3-(trifluoromethyl) phenyl)- 2-phenoxyethyl)- 5-oxopyrrolidine-3- carboxamide | [M + Na]⁺ 433.1 | [M + Na]⁺ 433.3 |
| 26 | 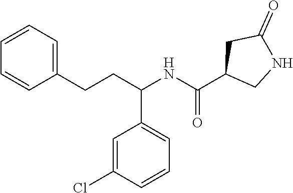 [SMILES: O=C1C[C@H](C(=O)NC(CCc2ccccc2)c2cccc(Cl)c2)CN1] | (3S)-N-(1-(3- chlorophenyl)-3- phenylpropyl)-5- oxopyrrolidine-3- carboxamideNa | [M + Na]⁺ 379.1 | [M + Na]⁺ 379.2 |

### Examples 27A, 27B, 27C and 27D

### (3R,4R)—N-(bis(4-chlorophenyl)methyl)-4-methyl-5-oxopyrrolidine-3-carboxamide, (3R,4S)—N-(bis(4-chlorophenyl)methyl)-4-methyl-5-oxopyrrolidine-3-carboxamide, (3S,4S)—N-(bis(4-chlorophenyl)methyl)-4-methyl-5-oxopyrrolidine-3-carboxamide and (3S,4R)—N-(bis(4-chlorophenyl)methyl)-4-methyl-5-oxopyrrolidine-3-carboxamide

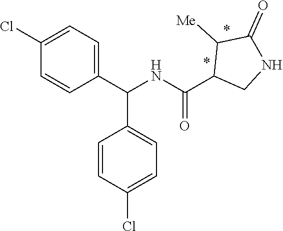 [SMILES: CC1C(=O)NCC1C(=O)NC(c1ccc(Cl)cc1)c1ccc(Cl)cc1]

Step 1: dimethyl 2-(bromomethyl)fumarate. A mixture of NBS (1.7 g, 9.5 mmol), dimethyl 2-methylfumarate (1.0 g, 6.3 mmol) and AIBN (0.021 g, 0.13 mmol) in CCl₄ (25 mL) was stirred at 85° C. for 20 h. Then the mixture was filtered and the filtrate was concentrated in vacuo. The resulting residue was purified by silica gel chromatography (0-10% EtOAc:PE) to give the title compound.

Step 2: dimethyl 2-methyl-3-methylenesuccinate. To a mixture of HMPA (11 mL, 63 mmol) and dimethyl 2-(bromomethyl)fumarate (3.0 g, 13 mmol) in THF (20 mL) was added MeMgBr (5.9 mL, 18 mmol) at −20° C. under N₂. The mixture was stirred for 2 h at −20° C., and then quenched with saturated NH₄C₁. Then 1 M HCl was added to the mixture, followed by extraction with EtOAc. The organic layers were combined, dried over Na₂SO₄, filtered and concentrated in vacuo. The resulting residue was purified by silica gel chromatography (0-10% EtOAc:PE) to give the title compound.

Step 3: methyl 1-(3,4-dimethoxybenzyl)-4-methyl-5-oxopyrrolidine-3-carboxylate. To a mixture of dimethyl 2-methyl-3-methylenesuccinate (60 mg, 0.35 mmol) in toluene (3 mL) was added (2,4-dimethoxyphenyl)methanamine (61 mg, 0.37 mmol) at 20° C. The mixture was stirred at 120° C. under N₂ for 10 h, then concentrated in vacuo and purified by preparative silica gel TLC to give the title compound.

Step 4: 1-(3,4-dimethoxybenzyl)-4-methyl-5-oxopyrrolidine-3-carboxylic acid. To a mixture of methyl 1-(3,4-dimethoxybenzyl)-4-methyl-5-oxopyrrolidine-3-carboxylate (0.45 g, 1.5 mmol) in THF (4 mL) and MeOH (4 mL) was added a solution of NaOH (0.29 g, 7.3 mmol, 2 mL in water). The reaction mixture was stirred at rt for 12 h, then diluted with water and extracted with PE. The aqueous layer was adjusted pH to 4-5 with 1 M HCl. The aqueous layer was extracted with EtOAc. Then the combined EtOAc layers were dried over Na₂SO₄, filtered and concentrated to give the title compound.

Step 5: N-(bis(4-chlorophenyl)methyl)-1-(3,4-dimethoxybenzyl)-4-methyl-5-oxopyrrolidine-3-carboxamide. To a solution of bis(4-chlorophenyl)methanamine (0.27 g, 1.1 mmol), 1-(3,4-dimethoxybenzyl)-4-methyl-5-oxopyrrolidine-3-carboxylic acid (0.31 g, 1.1 mmol) and N-ethyl-N-isopropylpropan-2-amine (0.68 g, 5.3 mmol) in DMF (5 mL) was added T3P® (1.0 g, 3.2 mmol) at 20° C. The resulting mixture was stirred at 40° C. for 12 h, then concentrated in vacuo. The resulting residue was purified by reverse phase HPLC (70:30 to 40:60; water (0.1% TFA):MeCN (0.1% TFA)), followed by lyophilization to give the title compound.

Step 6: (3(R and S),4(R and S)—N-(bis(4-chlorophenyl)methyl)-4-methyl-5-oxopyrrolidine-3-carboxamide. A mixture of N-(bis(4-chlorophenyl)methyl)-1-(3,4-dimethoxybenzyl)-4-methyl-5-oxopyrrolidine-3-carboxamide (0.25 g, 0.48 mmol) in TFA (5 mL) was stirred at 60° C. for 12 h. Then the mixture was concentrated in vacuo. The resulting residue was purified by reverse phase HPLC (95:5 to 5:95; water (0.1% TFA):MeCN (0.1% TFA)), followed by lyophilization to give the title compound.

Step 7: (3(R or S),4(R or S)—N-(bis(4-chlorophenyl)methyl)-4-methyl-5-oxopyrrolidine-3-carboxamide. (3(R and S),4(R and S)—N-(bis(4-chlorophenyl)methyl)-4-methyl-5-oxopyrrolidine-3-carboxamide was separated by chiral-SFC (method A), followed by chiral-SFC (method H) to give the title compounds: the first eluted diastereomer 27A (3(R or S),4(R or S)—N-(bis(4-chlorophenyl)methyl)-4-methyl-5-oxopyrrolidine-3-carboxamide, the second eluted diastereomer 27B (3(R or S),4(R or S)—N-(bis(4-chlorophenyl)methyl)-4-methyl-5-oxopyrrolidine-3-carboxamide, the third eluted diastereomer 27C (3(R or S),4(R or S)—N-(bis(4-chlorophenyl)methyl)-4-methyl-5-oxopyrrolidine-3-carboxamide, and the fourth eluted diastereomer 27D (3(R or S),4(R or S)—N-(bis(4-chlorophenyl)methyl)-4-methyl-5-oxopyrrolidine-3-carboxamide. Diastereomer 27A: LRMS m/z (M+H): calculated 377.1, observed 377.1. ¹H NMR (CD₃OD, 500 MHz) δ 8.99 (d, J=8.4 Hz, 1H), 7.43-7.34 (m, 4H), 7.31-7.22 (m, 4H), 6.22 (d, J=8.4 Hz, 1H), 3.66-3.56 (m, 1H), 3.51-3.41 (m, 2H), 2.82-2.67 (m, 1H), 1.02 (d, J=7.5 Hz, 3H). Diastereomer 27B: LRMS m/z (M+H): calculated 377.1, observed 377.1. ¹H NMR (CD₃OD, 500 MHz) δ 7.42-7.35 (m, 4H), 7.30-7.18 (m, 4H), 6.22 (s, 1H), 3.58-3.47 (m, 1H), 3.40 (dd, J=9.7, 8.5 Hz, 1H), 3.09-2.92 (m, 1H), 2.71 (qd, J=9.5, 7.2 Hz, 1H), 1.20 (d, J=7.2 Hz, 3H). Diastereomer 27C: LRMS m/z (M+H): calculated 377.1, observed 377.1. ¹H NMR (CD₃OD, 500 MHz) δ 7.39 (dd, J=8.1, 5.2 Hz, 4H), 7.26 (dd, J=8.5, 2.9 Hz, 4H), 6.22 (s, 1H), 3.58-3.48 (m, 1H), 3.40 (t, J=9.1 Hz, 1H), 3.01 (q, J=8.7 Hz, 1H), 2.78-2.63 (m, 1H), 1.20 (d, J=7.2 Hz, 3H). Diastereomer 27D: LRMS m/z (M+H): calculated 377.1, observed 377.1. ¹H NMR (CD₃OD, 500 MHz) δ 8.99 (d, J=8.4 Hz, 1H), 7.42-7.33 (m, 4H), 7.30-7.19 (m, 4H), 6.21 (d, J=8.2 Hz, 1H), 3.64-3.54 (m, 1H), 3.48-3.40 (m, 2H), 2.81-2.67 (m, 1H), 1.02 (d, J=7.5 Hz, 3H).

### Example of a Pharmaceutical Composition

As a specific embodiment of an oral pharmaceutical composition, a 100 mg potency tablet is composed of 100 mg of any one of the Examples, 268 mg microcrystalline cellulose, 20 mg of croscarmellose sodium, and 4 mg of magnesium stearate. The active, microcrystalline cellulose, and croscarmellose are blended first. The mixture is then lubricated by magnesium stearate and pressed into tablets.

### Biological Assays

### Qube® Assay Experimental Procedure

Compounds were tested on human Naᵥ1.8 and Naᵥ1.5 channels stably expressed in human embryo kidney (HEK) 293 cells. Sodium current measurements on Qube® were conducted as follows: automated 384-well patch-clamp assays on the Qube® platform (Sophion Biosciences) were used to measure the inhibition of sodium flow through human Nav1.8 and Nav1.5 channels. Whole-cell voltage-clamp recordings were performed in QChips® (Sophion Biosciences) at room temperature. Nav1.8 current measurements on Qube® were obtained as follows: Nav1.8 currents were elicited with a 10 second 1 Hertz (Hz) pulse train from a holding potential of −90 millivolts (mV), delivered to the cells once per minute in the control condition (DMSO only) and after compound addition. The 1 hertz pulse train stimulation consisted of ten test pulses to 10 millivolt (mV) for 20 milliseconds (ms), each of which was followed by a 980 millisecond repolarization to −67 millivolts. At the end of the 10 second pulse train stimulation, a 5 second hyperpolarization step to −100 millivolt (mV) was used to recover Naᵥ1.8 from fast inactivation. The peak currents elicited by the 1^{st} and 10^{th} test pulses were used to determine IC₅₀ values for resting inhibition and inactivated state inhibition. Naᵥ1.5 current measurements on Qube® were obtained as follows: Nav1.5 currents were elicited with a 20 second 3 Hertz pulse train in the control condition (DMSO only) and after compound addition. The pulse train consisted of sixty 20 millisecond test pulses to 0 millivolt from a holding potential of −80 millivolt (mV). The average peak currents elicited by the last 3 test pulses were used to determine IC₅₀ values for Nav1.5 inhibition.

The following buffers were used for the Qube® recordings: External buffer for Nav1.8 Qube® recording: 150 NaCl, 2 CaCl₂, 5 KCl, 1 Mg Cl₂, 10 HEPES, 12 Dextrose; External buffer for Qube® Nav1.5 recording: 120 N-Methyl-D-Glucamine, 40 NaCl, 1 KCl, 2.7 CaCl₂, 5 HEPES, 0.5 MgCl₂; and Internal buffer for Qube® recording: 120 CsF, 30 CsCl, 10 EGTA, 5 HEPES, 5 NaF, 2 MgCl₂.

For all Qube® experiments offline analysis was used to determine percent inhibition as a function of drug concentration. IC₅₀ values were determined by fitting to the Hill equation.

The compounds of the present invention have Nav1.8 IC₅₀ values in the Qube® Assay of less than 25 micromolar. Preferred compounds of the present invention have Nav1.8 IC₅₀ values in the Qube® Assay of less than 5 micromolar. More preferred compounds of the present invention have Nav1.8 IC₅₀ values in the Qube® Assay of less than 1 micromolar. Specific IC₅₀ values of the compounds of Examples 1A-27D in the Qube® Assay are listed in Table I.

TABLE I
IC50 values (nM) for Examples in the Nav1.8 Qube ® Assay

| Example | IC₅₀ (nM) |
|---|---|
| 1A | 3.4 |
| 1B | 13 |
| 2 | 820 |
| 3 | 186 |
| 4 | 108 |
| 5 | 11 |
| 6 | 520 |
| 7 | 186 |
| 8 | 19 |
| 9 | 28 |
| 10 | 52 |
| 11A | 2.6 |
| 11B | 17 |
| 12A | 28 |
| 12B | 4.1 |
| 13A | 139 |
| 13B | 129 |
| 14A | 1.2 |
| 14B | 9.4 |
| 15 | 4.2 |
| 16 | 46 |
| 17 | 11 |
| 18 | 5.3 |
| 19A | 16 |
| 19B | 17 |
| 20A | 51 |
| 20B | 2.9 |
| 21A | 2220 |
| 21B | 17 |
| 22 | 32 |
| 23 | 92 |
| 24 | 5.5 |
| 25 | 53 |
| 26 | 435 |
| 27A | 7460 |
| 27B | 277 |
| 27C | 3480 |
| 27D | 7650 |

The scope of the claims should not be limited by the preferred embodiments set forth in the examples, but should be given the broadest interpretation consistent with the description as a whole. While the invention has been described and illustrated with reference to certain particular embodiments thereof, those skilled in the art will appreciate that various adaptations, changes, modifications, substitutions, deletions, or additions of procedures and protocols may be made without departing from the scope of the invention. For example, effective dosages other than the particular dosages as set forth herein above may be applicable as a consequence of variations in responsiveness of the mammal being treated for any of the indications with the compounds of the invention indicated above. The specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention.

1. A compound of structural Formula I: 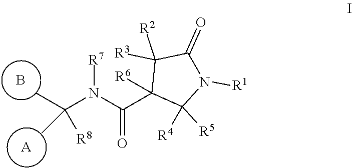
or a pharmaceutically acceptable salt thereof, wherein
one of A and B is selected from:
1) aryl, and
2) heteroaryl,
wherein aryl and heteroaryl are unsubstituted or substituted with one to five substituents selected from R^{a},
and the other of A and B is selected from:
1) aryl,
2) heteroaryl,
3) —C₁₋₆alkyl-aryl,
4) —C₃₋₈cycloalkyl-aryl,
5) —C₂₋₈cycloheteroalkyl-aryl,
6) —C₁₋₆alkyl-heteroaryl,
7) —C₃₋₈cycloalkyl-heteroaryl,
8) —C₂₋₈cycloheteroalkyl-heteroaryl,
9) —C₁₋₆alkyl-O-aryl,
10) —C₁₋₆alkyl-O-heteroaryl,
11) —C₃₋₁₂cycloalkyl,
12) —C₂₋₁₂cycloheteroalkyl,
13) —C₁₋₆alkyl-C₃₋₁₂cycloalkyl,
14) —C₁₋₆alkyl-C₂₋₁₂cycloheteroalkyl,
15) —C₁₋₆alkyl-O—C₃₋₁₂cycloalkyl,
16) —C₁₋₆alkyl-O—C₂₋₁₂cycloheteroalkyl,
17) —C₀₋₆alkyl-aryl fused to C₄₋₆cycloalkyl or C₄₋₆cycloheteroalkyl containing 1-3 heteroatoms independently selected from O, S and N(R^{h})₂,
18) —C₀₋₆alkyl-aryl fused to C₄₋₆cycloalkenyl or C₄₋₆cycloheteroalkenyl containing 1-3 heteroatoms independently selected from O, S and N(R^{h})₂,
19) —C₀₋₆alkyl-heteroaryl fused to C₄₋₆cycloalkyl or C₄₋₆cycloheteroalkyl containing 1-3 heteroatoms independently selected from O, S and N(R^{h})₂, and
20) —C₀₋₆alkyl-heteroaryl fused to C₄₋₆cycloalkenyl or C₄₋₆cycloheteroalkenyl containing 1-3 heteroatoms independently selected from O, S and N(R^{h})₂,
wherein alkyl, cycloalkyl, cycloheteroalkyl, cycloalkenyl, aryl and heteroaryl are unsubstituted or substituted with one to five substituents selected from R^{b};
R¹ is selected from the group consisting of:
1) hydrogen,
2) —C₁₋₆alkyl,
3) —C₃₋₆alkenyl,
4) —C₃₋₆alkynyl,
5) —C₃₋₁₀cycloalkyl,
6) —C₂₋₁₀cycloheteroalkyl,
7) —C₁₋₆alkyl-O—C₁₋₆alkyl-,
8) —(CH₂)ₛC(O)R^{j},
9) —(CH₂)ₛC(O)NR^{e}R^{j},
10) —(CH₂)ₙNR^{e}C(O)R^{j},
11) —(CH₂)ₙNR^{e}C(O)OR^{j},
12) —(CH₂)ₙNR^{e}C(O)N(R^{e})₂,
13) —(CH₂)ₙNR^{e}C(O)NR^{e}R^{j},
14) —(CH₂)ₙNR^{e}S(O)ₘR^{j},
15) —(CH₂)ₙNR^{e}S(O)mN(R^{e})₂,
16) —(CH₂)ₙNR^{e}S(O)mNR^{e}R^{j}, and
17) —(CH₂)ₙNR^{e}R^{j},
wherein each CH₂, alkyl, alkenyl, alkynyl, cycloalkyl and cycloheteroalkyl is unsubstituted or substituted with one to five substituents selected from R^{c};
R² is selected from the group consisting of:
1) hydrogen,
2) —C₁₋₆alkyl,
3) —C₂₋₆alkenyl,
4) —C₂₋₆alkynyl,
5) —C₃₋₁₀cycloalkyl,
6) —C₂₋₁₀cycloheteroalkyl,
7) —C₁₋₆alkyl-O—C₁₋₆alkyl-,
8) —(CH₂)ₛC(O)R^{j},
9) —(CH₂)ₛC(O)NR^{e}R^{j},
10) —(CH₂)ₛNR^{e}C(O)R^{j},
11) —(CH₂)ₛNR^{e}C(O)OR^{j},
12) —(CH₂)ₛNR^{e}C(O)N(R^{e})₂,
13) —(CH₂)ₛNR^{e}C(O)NR^{e}R^{j},
14) —(CH₂)ₛNR^{e}S(O)ₘR^{j},
15) —(CH₂)ₛNR^{e}S(O)mN(R^{e})₂,
16) —(CH₂)ₛNR^{e}S(O)mNR^{e}R^{j}, and
17) —(CH₂)ₛNR^{e}R^{j},
wherein each CH₂, alkyl, alkenyl, alkynyl, cycloalkyl, and cycloheteroalkyl is unsubstituted or substituted with one to five substituents selected from R^{d}, and wherein R² and R³ and the carbon atom they are connected to can form a —C₃₋₅cycloalkyl ring;
R³ is selected from the group consisting of:
1) hydrogen,
2) —C₁₋₆alkyl,
3) —C₂₋₆alkenyl,
4) —C₂₋₆alkynyl,
5) —C₃₋₁₀cycloalkyl,
6) —C₂₋₁₀cycloheteroalkyl,
7) —C₁₋₆alkyl-O—C₁₋₆alkyl-,
8) —(CH₂)ₛC(O)R^{j},
9) —(CH₂)ₛC(O)NR^{e}R^{j},
10) —(CH₂)ₛNR^{e}C(O)R^{j},
11) —(CH₂)ₛNR^{e}C(O)OR^{j},
12) —(CH₂)ₛNR^{e}C(O)N(R^{e})₂,
13) —(CH₂)ₛNR^{e}C(O)NR^{e}R^{j},
14) —(CH₂)ₛNR^{e}S(O)ₘR^{j},
15) —(CH₂)ₛNR^{e}S(O)mN(R^{e})₂,
16) —(CH₂)ₛNR^{e}S(O)mNR^{e}R^{j}, and
17) —(CH₂)ₛNR^{e}R^{j},
wherein each CH₂, alkyl, alkenyl, alkynyl, cycloalkyl, and cycloheteroalkyl is unsubstituted or substituted with one to five substituents selected from R^{f};
R⁴ is selected from the group consisting of:
1) hydrogen,
2) —C₁₋₆alkyl,
3) —C₂₋₆alkenyl,
4) —C₂₋₆alkynyl,
5) —C₃₋₁₀cycloalkyl,
6) —C₂₋₁₀cycloheteroalkyl,
7) —C₁₋₆alkyl-O—C₁₋₆alkyl-,
8) —(CH₂)ₛC(O)R^{j},
9) —(CH₂)ₛC(O)NR^{e}R^{j},
10) —(CH₂)ₛNR^{e}C(O)R^{j},
11) —(CH₂)ₛNR^{e}C(O)OR^{j},
12) —(CH₂)ₛNR^{e}C(O)N(R^{e})₂,
13) —(CH₂)ₛNR^{e}C(O)NR^{e}R^{j},
14) —(CH₂)ₛNR^{e}S(O)ₘR^{j},
15) —(CH₂)ₛNR^{e}S(O)mN(R^{e})₂,
16) —(CH₂)ₛNR^{e}S(O)mNR^{e}R^{j}, and
17) —(CH₂)ₛNR^{e}R^{j},
wherein each CH₂, alkyl, alkenyl, alkynyl, cycloalkyl, and cycloheteroalkyl is unsubstituted or substituted with one to five substituents selected from R^{g};
R⁵ is selected from the group consisting of:
1) hydrogen,
2) —C₁₋₆alkyl,
3) —C₂₋₆alkenyl,
4) —C₂₋₆alkynyl,
5) —C₃₋₁₀cycloalkyl,
6) —C₂₋₁₀cycloheteroalkyl,
7) —C₁₋₆alkyl-O—C₁₋₆alkyl-,
8) —(CH₂)ₛC(O)R^{j},
9) —(CH₂)ₛC(O)NR^{e}R^{j},
10) —(CH₂)ₛNR^{e}C(O)R^{j},
11) —(CH₂)ₛNR^{e}C(O)OR^{j},
12) —(CH₂)ₛNR^{e}C(O)N(R^{e})₂,
13) —(CH₂)ₛNR^{e}C(O)NR^{e}R^{j},
14) —(CH₂)ₛNR^{e}S(O)ₘR^{j},
15) —(CH₂)ₛNR^{e}S(O)mN(R^{e})₂,
16) —(CH₂)ₛNR^{e}S(O)mNR^{e}R^{j}, and
17) —(CH₂)ₛNR^{e}R^{j},
wherein each CH₂, alkyl, alkenyl, alkynyl, cycloalkyl, and cycloheteroalkyl is unsubstituted or substituted with one to five substituents selected from R^{g}, and
wherein R⁵ and R⁴ and the carbon atom they are connected to can form a —C₃₋₅cycloalkyl ring, or wherein R⁵ and R⁶ and the carbon atoms they are connected to can form a —C₃₋₅cycloalkyl ring;
R⁶ is selected from the group consisting of:
1) hydrogen, and
2) —C₁₋₆alkyl,
wherein each alkyl is unsubstituted or substituted with one to five substituents selected from halogen;
R⁷ is selected from the group consisting of:
1) hydrogen,
2) —C₁₋₆alkyl,
3) —C₃₋₆cycloalkyl, and
4) —C₂₋₆cycloheteroalkyl,
wherein each alkyl, cycloalkyl and cycloheteroalkyl is unsubstituted or substituted with one to five substituents selected from halogen;
R⁸ is selected from the group consisting of:
1) hydrogen,
2) —C₁₋₆alkyl,
3) —C₂₋₆alkenyl, and
4) —C₂₋₆alkynyl,
wherein each alkyl, alkenyl and alkynyl is unsubstituted or substituted with one to five halogen substituents;
each R^{a} is independently selected from the group consisting of:
1) —CF₃,
3) —CHF₂,
4) —OCHF₂,
5) —CH₂CF₃,
6) —OCH₂CF₃,
7) —CF₂CH₃
8) CN,
9) oxo,
10) halogen,
11) —S(O)₂C₁₋₆alkyl,
12) —C₁₋₆alkyl,
13) —C₂₋₆alkenyl,
14) —C₂₋₆alkynyl,
15) —C₃₋₆cycloalkyl,
16) —C₂₋₆cycloheteroalkyl,
17) aryl,
18) heteroaryl,
19) —C₁₋₆alkyl-aryl,
20) —C₁₋₆alkyl-heteroaryl,
21) —C₁₋₆alkyl-C₃₋₆cycloalkyl,
22) —C₁₋₆alkyl-C₂₋₆cycloheteroalkyl,
23) —C₂₋₆alkenyl-C₃₋₆cycloalkyl,
24) —C₂₋₆alkenyl-C₂₋₆cycloheteroalkyl,
25) —C₂₋₆alkenyl-aryl,
26) —C₂₋₆alkenyl-heteroaryl,
27) —C₂₋₆alkynyl-C₃₋₆cycloalkyl,
28) —C₂₋₆alkynyl-C₂₋₆cycloheteroalkyl,
29) —C₂₋₆alkynyl-aryl,
30) —C₂₋₆alkynyl-heteroaryl,
31) —OH,
32) —(CH₂)ₚ—OC₁₋₆alkyl,
33) —(CH₂)ₚ—OC₂₋₆alkenyl,
34) —(CH₂)ₚ—OC₂₋₆alkynyl,
35) —(CH₂)ₚ—OC₃₋₆cycloalkyl,
36) —(CH₂)ₚ—OC₂₋₆heterocycloalkyl,
37) —(CH₂)ₚ—O-aryl,
38) —(CH₂)ₚ—O-heteroaryl,
39) —OC₁₋₆alkyl-C₃₋₆cycloalkyl,
40) —OC₁₋₆alkyl-C₂₋₆ heterocycloalkyl,
41) —OC₁₋₆alkyl-aryl,
42) —OC₁₋₆alkyl-heteroaryl,
43) —S(O)ₘRⁱ,
44) —C₁₋₆alkyl-S(O)ₘRⁱ,
45) —N(R^{k})₂, and
46) —NR^{k}R^{L},
wherein each R^{a} is unsubstituted or substituted with one to six substituents selected from halogen, CF₃, OH, C₁₋₆alkyl, and —OC₁₋₆alkyl;
each R^{b} is independently selected from the group consisting of:
1) —CF₃,
2) —OCF₃,
3) —CHF₂,
4) —OCHF₂,
5) —CH₂CF₃,
6) —OCH₂CF₃,
7) —CF₂CH₃
8) CN,
9) oxo,
10) halogen,
11) —S(O)₂C₁₋₆alkyl,
12) —C₁₋₆alkyl,
13) —C₂₋₆alkenyl,
14) —C₂₋₆alkynyl,
15) —O—C₁₋₆alkyl,
16) —C₃₋₆cycloalkyl,
17) —O—C₃₋₆cycloalkyl,
18) —C₂₋₆cycloheteroalkyl,
19) aryl,
20) heteroaryl,
21) —C₁₋₆alkyl-aryl,
22) —C₁₋₆alkyl-heteroaryl,
23) —C₁₋₆alkyl-C₃₋₆cycloalkyl,
24) —C₁₋₆alkyl-C₂₋₆cycloheteroalkyl,
25) —C₂₋₆alkenyl-C₃₋₆cycloalkyl,
26) —C₂₋₆alkenyl-C₂₋₆cycloheteroalkyl,
27) —C₂₋₆alkenyl-aryl,
28) —C₂₋₆alkenyl-heteroaryl,
29) —C₂₋₆alkynyl-C₃₋₆cycloalkyl,
30) —C₂₋₆alkynyl-C₂₋₆cycloheteroalkyl,
31) —C₂₋₆alkynyl-aryl,
32) —C₂₋₆alkynyl-heteroaryl,
33) —OH,
34) —(CH₂)_{q}—OC₁₋₆alkyl,
35) —(CH₂)_{q}—OC₂₋₆alkenyl,
36) —(CH₂)_{q}—OC₂₋₆alkynyl,
37) —(CH₂)_{q}—OC₃₋₆cycloalkyl,
38) —(CH₂)_{q}—OC₂₋₆heterocycloalkyl,
39) —(CH₂)_{q}—O-aryl,
40) —(CH₂)_{q}—O-heteroaryl,
41) —OC₁₋₆alkyl-C₃₋₆cycloalkyl,
42) —OC₁₋₆alkyl-C₂₋₆heterocycloalkyl,
43) —OC₁₋₆alkyl-aryl,
44) —OC₁₋₆alkyl-heteroaryl,
45) —S(O)ₘR^{j},
46) —C₁₋₆alkyl-S(O)ₘR^{j},
47) —C(O)R^{L}, and
48) —NR^{k}R^{L},
wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl;
R^{c} is selected from:
1) —C₁₋₆alkyl,
2) OH,
3) halogen, and
4) —OC₁₋₆alkyl,
wherein alkyl can be unsubstituted or substituted with one to three halogens;
R^{d} is selected from:
1) —C₁₋₆alkyl,
2) OH,
3) halogen, and
4) —OC₁₋₆alkyl,
wherein alkyl can be unsubstituted or substituted with one to three halogens;
R^{e} is selected from:
1) hydrogen, and
2) C₁₋₆alkyl;
R^{f} is selected from:
1) —C₁₋₆alkyl,
2) OH,
3) halogen, and
4) —OC₁₋₆alkyl,
wherein alkyl can be unsubstituted or substituted with one to three halogens;
R^{g} is selected from:
1) —C₁₋₆alkyl,
2) OH,
3) halogen, and
4) —OC₁₋₆alkyl,
wherein alkyl can be unsubstituted or substituted with one to three halogens;
R^{h} is selected from:
1) hydrogen, and
2) C₁₋₆alkyl;
Rⁱ is selected from:
1) hydrogen,
2) C₁₋₆alkyl,
3) C₃₋₆cycloalkyl,
4) aryl, and
5) heteroaryl;
R^{j} is selected from:
1) hydrogen,
2) C₁₋₆alkyl,
3) C₃₋₆alkenyl,
4) C₃₋₆alkynyl,
5) C₃₋₆cycloalkyl,
6) C₂₋₅cycloheteroalkyl,
7) aryl, and
8) heteroaryl;
R^{k} is selected from:
1) hydrogen, and
2) C₁₋₆alkyl;
R^{L} is selected from:
1) hydrogen,
2) C₁₋₆alkyl,
3) C₃₋₆cycloalkyl,
4) aryl, and
5) heteroaryl;
m is independently selected from 0 to 2;
n is independently selected from 2 to 6;
p is independently selected from 0 to 3;
q is independently selected from 0 to 3;
r is independently selected from 0 to 2; and
s is independently selected from 0 to 6.

2. The compound according to claim 1 wherein A is selected from the group consisting of:
1) aryl, and
2) heteroaryl,
wherein aryl and heteroaryl are unsubstituted or substituted with one to five substituents selected from R^{a}; or a pharmaceutically acceptable salt thereof.

3. The compound according to claim 1 wherein A is aryl, wherein aryl is unsubstituted or substituted with one to five substituents selected from R^{a}; or a pharmaceutically acceptable salt thereof.

4. The compound according to claim 1 wherein A is phenyl, wherein phenyl is unsubstituted or substituted with one to three substituents selected from R^{a}; or a pharmaceutically acceptable salt thereof.

5. The compound according to claim 1 wherein B is independently selected from the group consisting of:
1) aryl,
2) heteroaryl,
3) —C₁₋₆alkyl-aryl,
4) —C₃₋₈cycloalkyl-aryl,
5) —C₂₋₈cycloheteroalkyl-aryl,
6) —C₁₋₆alkyl-heteroaryl,
7) —C₃₋₈cycloalkyl-heteroaryl,
8) —C₂₋₈cycloheteroalkyl-heteroaryl,
9) —C₁₋₆alkyl-O-aryl,
10) —C₁₋₆alkyl-O-heteroaryl,
11) —C₃₋₁₂cycloalkyl,
12) —C₂₋₁₂cycloheteroalkyl,
13) —C₁₋₆alkyl-C₃₋₁₂cycloalkyl,
14) —C₁₋₆alkyl-C₂₋₁₂cycloheteroalkyl,
15) —C₁₋₆alkyl-O—C₃₋₁₂cycloalkyl,
16) —C₁₋₆alkyl-O—C₂₋₁₂cycloheteroalkyl,
17) —C₀₋₆alkyl-aryl fused to C₄₋₆cycloalkyl or C₄₋₆cycloheteroalkyl containing 1-3 heteroatoms independently selected from O, S and N(R^{h})₂,
18) —C₀₋₆alkyl-aryl fused to C₄₋₆cycloalkenyl or C₄₋₆cycloheteroalkenyl containing 1-3 heteroatoms independently selected from O, S and N(R^{h})₂,
19) —C₀₋₆alkyl-heteroaryl fused to C₄₋₆cycloalkyl or C₄₋₆cycloheteroalkyl containing 1-3 heteroatoms independently selected from O, S and N(R^{h})₂, and
20) —C₀₋₆alkyl-heteroaryl fused to C₄₋₆cycloalkenyl or C₄₋₆cycloheteroalkenyl containing 1-3 heteroatoms independently selected from O, S and N(R^{h})₂,
wherein alkyl, cycloalkyl, cycloheteroalkyl, cycloalkenyl, aryl and heteroaryl are unsubstituted or substituted with one to five substituents selected from R^{b};
or a pharmaceutically acceptable salt thereof.

6. The compound according to claim 1 wherein B is independently selected from the group consisting of:
1) aryl,
2) heteroaryl,
3) —C₁₋₆alkyl-aryl,
4) —C₁₋₆alkyl-O-aryl, and
5) —C₃₋₁₂cycloalkyl,
wherein alkyl, cycloalkyl, aryl and heteroaryl are unsubstituted or substituted with one to five substituents selected from R^{b}; or a pharmaceutically acceptable salt thereof.

7. The compound according to claim 1 wherein B is independently selected from the group consisting of:
1) phenyl,
2) pyridinyl,
3) thiazolyl,
4) —(CH₂)₂-phenyl,
5) —CH₂—O-phenyl, and
6) cyclobutanyl,
wherein B is unsubstituted or substituted with one to five substituents selected from R^{b}; or a pharmaceutically acceptable salt thereof.

8. The compound according to claim 1 wherein R⁶ is hydrogen; or a pharmaceutically acceptable salt thereof.

9. The compound according to claim 1 wherein R¹ is selected from the group consisting of:
1) hydrogen, and
2) —C₁₋₆alkyl,
wherein each alkyl is unsubstituted or substituted with one to five substituents selected from R^{c};
R² is selected from the group consisting of:
1) hydrogen, and
2) —C₁₋₆alkyl,
wherein each alkyl is unsubstituted or substituted with one to five substituents selected from R^{d};
R³ is selected from the group consisting of:
1) hydrogen, and
2) —C₁₋₆alkyl,
wherein each alkyl is unsubstituted or substituted with one to five substituents selected from R^{F};
R⁴ is selected from the group consisting of:
1) hydrogen, and
2) —C₁₋₆alkyl,
wherein each alkyl is unsubstituted or substituted with one to five substituents selected from R^{g};
R⁵ is selected from the group consisting of:
1) hydrogen, and
2) —C₁₋₆alkyl,
wherein each alkyl is unsubstituted or substituted with one to five substituents selected from R^{g};
R⁷ is selected from the group consisting of:
1) hydrogen, and
2) —C₁₋₆alkyl,
wherein each alkyl is unsubstituted or substituted with one to five halogen substituents;
R⁸ is selected from the group consisting of:
1) hydrogen, and
2) —C₁₋₆alkyl,
wherein each alkyl is unsubstituted or substituted with one to five halogen substituents;
or a pharmaceutically acceptable salt thereof.

10. The compound according to claim 1 wherein R⁷ is hydrogen; or a pharmaceutically acceptable salt thereof.

11. The compound according to claim 1 wherein R¹, R², R³, R⁴, R⁵, and R⁸ are hydrogen; or a pharmaceutically acceptable salt thereof.

12. The compound according to claim 1 wherein each R^{a} is independently selected from the group consisting of:
1) —CF₃,
2) —OCF₃, and
3) halogen;
or a pharmaceutically acceptable salt thereof.

13. The compound according to claim 1 wherein each R^{a} is independently selected from the group consisting of:
1) —CF₃, and
2) halogen;
or a pharmaceutically acceptable salt thereof.

14. The compound according to claim 1 wherein each R^{b} is independently selected from the group consisting of:
1) —CF₃,
2) —OCF₃,
3) —OCHF₂,
4) CN,
5) halogen,
6) —C₁₋₆alkyl,
7) —O—C₁₋₆alkyl, and
8) —C₃₋₆cycloalkyl,
wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl;
or a pharmaceutically acceptable salt thereof.

15. The compound according to claim 1 wherein each R^{b} is independently selected from the group consisting of:
1) —CF₃, and
2) halogen;
or a pharmaceutically acceptable salt thereof.

16. The compound according to claim 1 wherein
A is aryl, wherein aryl is unsubstituted or substituted with one to five substituents selected from R^{a};
B is independently selected from the group consisting of:
1) aryl,
2) heteroaryl,
3) —C₁₋₆alkyl-aryl,
4) —C₁₋₆alkyl-O-aryl, and
5) —C₃₋₁₂cycloalkyl,
wherein alkyl, cycloalkyl, aryl and heteroaryl are unsubstituted or substituted with one to five substituents selected from R^{b};
R¹ is selected from the group consisting of:
1) hydrogen, and
2) —C₁₋₆alkyl,
wherein each alkyl is unsubstituted or substituted with one to five substituents selected from R^{c};
R² is selected from the group consisting of:
1) hydrogen, and
2) —C₁₋₆alkyl,
wherein each alkyl is unsubstituted or substituted with one to five substituents selected from R^{d};
R³ is selected from the group consisting of:
1) hydrogen, and
2) —C₁₋₆alkyl,
wherein each alkyl is unsubstituted or substituted with one to five substituents selected from R^{f};
R⁴ is selected from the group consisting of:
1) hydrogen, and
2) —C₁₋₆alkyl,
wherein each alkyl is unsubstituted or substituted with one to five substituents selected from R^{g};
R⁵ is selected from the group consisting of:
1) hydrogen, and
2) —C₁₋₆alkyl,
wherein each alkyl is unsubstituted or substituted with one to five substituents selected from R^{g};
R⁶ is hydrogen;
R⁷ is selected from the group consisting of:
1) hydrogen, and
2) —C₁₋₆alkyl,
wherein each alkyl is unsubstituted or substituted with one to five halogen substituents;
R⁸ is selected from the group consisting of:
1) hydrogen, and
2) C₁₋₆alkyl,
wherein each alkyl is unsubstituted or substituted with one to five halogen substituents;
each R^{a} is independently selected from the group consisting of:
1) —CF₃,
2) —OCF₃, and
3) halogen;
each R^{b} is independently selected from the group consisting of:
1) —CF₃,
2) —OCF₃,
3) —OCHF₂,
4) CN,
5) halogen,
6) —C₁₋₆alkyl,
7) —O—C₁₋₆alkyl, and
8) —C₃₋₆cycloalkyl,
wherein each R^{b} is unsubstituted or substituted with one to six substituents selected from halogen, CF₃, OCF₃, CN, CH₂CF₃, CF₂CH₃, —C₁₋₆alkyl, and —OC₁₋₆alkyl;
or a pharmaceutically acceptable salt thereof.

17. The compound according to claim 1 wherein
A is phenyl, wherein phenyl is unsubstituted or substituted with one to five substituents selected from R^{a};
B is independently selected from the group consisting of:
1) phenyl,
2) pyridinyl,
3) thiazolyl,
4) —(CH₂)₂-phenyl,
5) —CH₂—O-phenyl, and
6) cyclobutyl,
wherein B is unsubstituted or substituted with one to five substituents selected from R^{b};
R¹, R², R³, R⁴, R⁵, R⁶, R⁷ and R⁸ are hydrogen;
each R^{a} is independently selected from the group consisting of:
1) —CF₃, and
2) halogen;
each R^{b} is independently selected from the group consisting of:
1) —CF₃, and
2) halogen;
or a pharmaceutically acceptable salt thereof.

18. The compound according to claim 1 selected from:
1) (S—N—((R)-(3-chloro-4-fluorophenyl)(4-chlorophenyl)methyl)-5-oxopyrrolidine-3-carboxamide;
2) (S)—N—((S)-(3-chloro-4-fluorophenyl)(4-chlorophenyl)methyl)-5-oxopyrrolidine-3-carboxamide;
3) (3S)—N-((3-chlorophenyl)(3-cyanophenyl)methyl)-5-oxopyrrolidine-3-carboxamide;
4) (3S)—N-((4-chlorophenyl)(4-cyclopropylphenyl)methyl)-5-oxopyrrolidine-3-carboxamide;
5) (3S)—N-((4-chlorophenyl)(4-isopropylphenyl)methyl)-5-oxopyrrolidine-3-carboxamide;
6) (3S)—N-((4-chloro-2-methoxyphenyl)(4-chlorophenyl)methyl)-5-oxopyrrolidine-3-carboxamide;
7) (3S)—N-((3-chlorophenyl)(3-(difluoromethoxy)phenyl)methyl)-5-oxopyrrolidine-3-carboxamide;
8) (3 S)—N-((4-chlorophenyl)(3-fluoro-5-(trifluoromethyl)phenyl)methyl)-5-oxopyrrolidine-3-carboxamide;
9) (3S)-5-oxo-N-((3-(trifluoromethyl)phenyl)(4-(trifluoromethyl)phenyl)methyl)pyrrolidine-3-carboxamide;
10) (3S)-5-oxo-N-((3-(trifluoromethyl)phenyl)(4-(trifluoromethyl)phenyl)methyl)pyrrolidine-3-carboxamide;
11) (S)—N-(bis(4-(trifluoromethyl)phenyl)methyl)-5-oxopyrrolidine-3-carboxamide;
12) (3S)—N-((4-chlorophenyl)(3-(trifluoromethoxy)phenyl)methyl)-5-oxopyrrolidine-3-carboxamide;
13) (S—N—((R)-(4-chlorophenyl)(4-fluoro-3-(trifluoromethyl)phenyl)methyl)-5-oxopyrrolidine-3-carboxamide;
14) (S)—N—((S)-(4-chlorophenyl)(4-fluoro-3-(trifluoromethyl)phenyl)methyl)-5-oxopyrrolidine-3-carboxamide;
15) (S)—N—((R)-(4-chlorophenyl)(4-(trifluoromethoxy)phenyl)methyl)-5-oxopyrrolidine-3-carboxamide;
16) (S)—N—((S)-(4-chlorophenyl)(4-(trifluoromethoxy)phenyl)methyl)-5-oxopyrrolidine-3-carboxamide;
17) (S—N—((R)-(3-chloro-4-fluorophenyl)(4-cyanophenyl)methyl)-5-oxopyrrolidine-3-carboxamide;
18) (S)—N—((S)-(3-chloro-4-fluorophenyl)(4-cyanophenyl)methyl)-5-oxopyrrolidine-3-carboxamide;
19) (S—N—((R)-(4-chloro-3-(trifluoromethyl)phenyl)(4-chlorophenyl)methyl)-5-oxo-pyrrolidine-3-carboxamide;
20) (S)—N—((S)-(4-chloro-3-(trifluoromethyl)phenyl)(4-chlorophenyl)methyl)-5-oxo-pyrrolidine-3-carboxamide;
21) (S—N—((R)-(3-chloro-2,4-difluorophenyl)(5-chloro-6-(trifluoromethyl)pyridin-3-yl)methyl)-5-oxopyrrolidine-3-carboxamide;
22) (S)—N—((S)-(3-chloro-2,4-difluorophenyl)((trans)-3-(trifluoromethyl)cyclobutyl)-methyl)-5-oxopyrrolidine-3-carboxamide;
23) (S—N—((R)-(3-chloro-4-fluorophenyl)(4-(trifluoromethyl)phenyl)methyl)-5-oxo-pyrrolidine-3-carboxamide;
24) (S)—N—((S)-(3-chloro-4-fluorophenyl)(4-(trifluoromethyl)phenyl)methyl)-5-oxo-pyrrolidine-3-carboxamide;
25) (S)—N-(bis(3-chloro-4-fluorophenyl)methyl)-5-oxopyrrolidine-3-carboxamide;
26) (S—N—((R)-(3-chlorophenyl)(4-(trifluoromethoxy)phenyl)methyl)-5-oxopyrrolidine-3-carboxamide;
27) (S)—N—((S)-(3-chlorophenyl)(4-(trifluoromethoxy)phenyl)methyl)-5-oxopyrrolidine-3-carboxamide;
28) (S—N—((R)-(3-chloro-4-fluorophenyl)(5-chloro-6-(trifluoromethyl)-pyridin-2-yl)-methyl)-5-oxopyrrolidine-3-carboxamide;
29) (S)—N—((S)-(3-chloro-4-fluorophenyl)(5-chloro-6-(trifluoromethyl)-pyridin-2-yl)-methyl)-5-oxopyrrolidine-3-carboxamide;
30) (S—N—((R)-(3-chloro-2,4-difluorophenyl)(3,3-dimethylcyclobutyl)methyl)-5-oxo-pyrrolidine-3-carboxamide;
31) (S)—N—((S)-(3-chloro-2,4-difluorophenyl)(3,3-dimethylcyclobutyl)methyl)-5-oxo-pyrrolidine-3-carboxamide;
32) (3S)—N-((3-chloro-4-fluorophenyl)(3-cyano-4-fluorophenyl)methyl)-5-oxopyrrolidine-3-carboxamide;
33) (3S)—N-((4-fluoro-3-(trifluoromethyl)phenyl)(2-(trifluoromethyl)thiazol-4-yl)methyl)-5-oxopyrrolidine-3-carboxamide;
34) (3S)—N-((3-chloro-2,4-difluorophenyl)(4-fluoro-3-(trifluoromethyl)phenyl)methyl)-5-oxopyrrolidine-3-carboxamide;
35) (3S)—N-(1-(4-fluoro-3-(trifluoromethyl)phenyl)-2-phenoxyethyl)-5-oxopyrrolidine-3-carboxamide;
36) (3S)—N-(1-(3-chlorophenyl)-3-phenylpropyl)-5-oxopyrrolidine-3-carboxamide;
37) (3R,4R)—N-(bis(4-chlorophenyl)methyl)-4-methyl-5-oxopyrrolidine-3-carboxamide;
38) (3R,4S)—N-(bis(4-chlorophenyl)methyl)-4-methyl-5-oxopyrrolidine-3-carboxamide;
39) (3S,4S)—N-(bis(4-chlorophenyl)methyl)-4-methyl-5-oxopyrrolidine-3-carboxamide; and
40) (3S,4R)—N-(bis(4-chlorophenyl)methyl)-4-methyl-5-oxopyrrolidine-3-carboxamide;
or a pharmaceutically acceptable salt thereof.

19. The compound according to claim 1 selected from:
1) (S—N—((R)-(3-chloro-4-fluorophenyl)(4-chlorophenyl)methyl)-5-oxopyrrolidine-3-carboxamide;
2) (S)—N—((S)-(3-chloro-4-fluorophenyl)(4-chlorophenyl)methyl)-5-oxopyrrolidine-3-carboxamide;
3) (S—N—((R)-(4-chloro-3-(trifluoromethyl)phenyl)(4-chlorophenyl)methyl)-5-oxo-pyrrolidine-3-carboxamide; and
4) (S)—N—((S)-(4-chloro-3-(trifluoromethyl)phenyl)(4-chlorophenyl)methyl)-5-oxo-pyrrolidine-3-carboxamide;
or a pharmaceutically acceptable salt thereof.

20. A pharmaceutical composition comprising a compound of claim 1, or a pharmaceutically acceptable salt thereof, and a pharmaceutically acceptable carrier.

21. (canceled)

22. (canceled)

23. (canceled)

24. (canceled)

25. A compound according to claim 1, or a pharmaceutically acceptable salt thereof, for use in therapy.

26. A method of treating or preventing a disorder, condition or disease that is responsive to the inhibition of Naᵥ1.8 channel activity in a patient in need thereof comprising administration of a therapeutically effective amount of a compound according to claim 1, or a pharmaceutically acceptable salt thereof.

27. The method of claim 26 wherein the disorder is selected from: pain disorder, a cough disorder, an acute itch disorder or chronic itch disorder.

28. The method of claim 27 wherein the disorder is a pain disorder.

29. The method of claim 28 wherein the pain disorder is selected from: acute pain, inflammatory pain, or neuropathic pain.

30. The compound according to claim 1 which is: (S—N—((R)-(3-chloro-4-fluorophenyl)(4-chlorophenyl)methyl)-5-oxopyrrolidine-3-carboxamide; or a pharmaceutically acceptable salt thereof.

31. The compound according to claim 1 which is: (S)—N—((S)-(3-chloro-4-fluorophenyl)(4-chlorophenyl)methyl)-5-oxopyrrolidine-3-carboxamide; or a pharmaceutically acceptable salt thereof.

32. The compound according to claim 1 which is: (S—N—((R)-(4-chloro-3-(trifluoromethyl)phenyl)(4-chlorophenyl)methyl)-5-oxo-pyrrolidine-3-carboxamide; or a pharmaceutically acceptable salt thereof.

33. The compound according to claim 1 which is: (S)—N—((S)-(4-chloro-3-(trifluoromethyl)phenyl)(4-chlorophenyl)methyl)-5-oxo-pyrrolidine-3-carboxamide; or a pharmaceutically acceptable salt thereof.
